SEQUENCE LISTING (255 sequences)

SEQ ID NO: 1 - DNA, 200 bp, unidentified
  source 1..200: mol_type = unassigned DNA
accaggaattttggagaagttcccagcagctcaggagagtggctggtgccttgtcccaggggagagggaaataagcacaggcctatttctttctcaagacggaaaaaccgagccccagaaaatggcgataagatggggatgcaggaaaaagctagctaatgaacagttcagcagtgggcaccgtgtgggaacctctccta

SEQ ID NO: 2 - DNA, 200 bp, unidentified
  source 1..200: mol_type = unassigned DNA
cacaaagtttcaggggtgagtgtgacccagggttagcttctaggtctgctcaggccctaccccacactaaactcaaatatcacttcccctcaggagctcaggcgatgggagaggaagatgaagatggagtaggtaccacaggtaatgaaagaaacagcacagcgaaccgcagatatgcgggaggagaacggcagagctag

SEQ ID NO: 3 - DNA, 200 bp, unidentified
  source 1..200: mol_type = unassigned DNA
gggtgaaggcaggagacttacttcggagtaggaccctgccaaaaaggctgtgatccctgtccagggtgttgcaattccagcggtgctggcggaactggtgctggcattctgctgtccactcggccacgccctggctaatggcacgcatcacatctggatgtcggtgacacagctgccgctggctgctcaccaggcctggc

SEQ ID NO: 4 - DNA, 200 bp, unidentified
  source 1..200: mol_type = unassigned DNA
ctctgcctttttctgagtctgtttcctctcaccatctctctctccatccgtccctccctgtatctgcccctctctccatctcgctgctgtgtccagctctctctctcgctcattccttcctcgccatctggggatctccccggctccgtgtcccctgcattgccccgcgccccaatcaccggccaaagcgcccgcggagc

SEQ ID NO: 5 - DNA, 200 bp, unidentified
  source 1..200: mol_type = unassigned DNA
tctctcccaacccgcccccccaccacacacaggcaggctgtcagtcaccctgaaacaataaggcttttcaaaagaggaaaatcaagcttaaacgctggagaggaacagactaaaaacttaggggtcaaaggctcatagactgttgattcatgtgttcaggctagaaagggctgtggatgtgcatgccacctccaggttct

SEQ ID NO: 6 - DNA, 200 bp, unidentified
  source 1..200: mol_type = unassigned DNA
tgtccaatcagtctgactaatctccctgaaatgccaccttgggcatttcttcagagccctctcaggcccaggaactggactgggctggtccgccttgcaatgccctctgtggcctggccccaccctccatgcacacgccctttgcacctcattgactggcatcctgccatttctcctgcagatgctatacctcttacctc

SEQ ID NO: 7 - DNA, 200 bp, unidentified
  source 1..200: mol_type = unassigned DNA
gcggacatgcatcactagcccataatttaggcaattttgtttaaagaccactggtatttcctgggggaattaatcgccggctccaagagtgctttgggtcattaatcttatattcttattaaattctcaggaaatgactatcttatgaggagggagcactgcagagattagagaaatgcacactgggagagcatgggtac

SEQ ID NO: 8 - DNA, 200 bp, unidentified
  source 1..200: mol_type = unassigned DNA
ggcgtcaccgccgccttccccctgagtccctccttcctgcgtctggtcattcattcattcactggctcagtccggtccttcttccttcacttctcattcactcagacggctgccttattttctcggcagttaggtgaccttggaccagtcaaccaacctcacctagcctcagtttgttagaaaaacaaggggggaggtgg

SEQ ID NO: 9 - DNA, 200 bp, unidentified
  source 1..200: mol_type = unassigned DNA
ggagatgccactgtccgcttggtttaatgatcaatgagctccctgccaggaaaccctttctgacctggtttgcccctcagtccctcgggctcatacctagtgcctgcggcaggacagccatggccgccaactccaccagcgacctccacactcccgggacgcagctgagcgtggctgacatcatcgtcatcactgtgtat

SEQ ID NO: 10 - DNA, 200 bp, unidentified
  source 1..200: mol_type = unassigned DNA
gtgtttttaaacagaaacataaagggaatggaagccagggtggctaatggtacataatggccttaattacaccactgaaggatcaatattagcgtagctcagtcaccagcagccaaggcacagaaactggtacagcaggaagtgggaaactgggaatcggtcatccccgcctgatgctcagggtgcacattccaaaaaga

SEQ ID NO: 11 - DNA, 200 bp, unidentified
  source 1..200: mol_type = unassigned DNA
tttgatctctagatattctactttggtcaataaagcagaagtcagagtgatttcctcattctgtggtctgttcttctattgcataaaataccgaagtgccacctcctggctaccacaggtgctatcagccaggcgggcaggcagcatgaacaaggcaaccactgggggatggcgcagtgccacacggcctgtggtaggga

SEQ ID NO: 12 - DNA, 200 bp, unidentified
  source 1..200: mol_type = unassigned DNA
atctatgtctatttcacccaagaactcacctttggatttgcacaactatatcgattgacctcttaattgagtgtttttgaaatatttaaccaggaggactataaccagtgtatctaatagtttggtgatcatatcttgagtaaccagttgattctctgccatgcctgaagacttggaattctgctttaaaacacagttac

SEQ ID NO: 13 - DNA, 200 bp, unidentified
  source 1..200: mol_type = unassigned DNA
aactgggttccctgacacccagagctgctctgaaatggccagcggcttcacgcataacaaagaccttgccccttggcattgtttattccttgacctgtgcagagagtccttcaaaagaggctttactgaaactatttcagggttgctttccaggggcagaattttctaaagagaaaaattatttatgcatatagaaagtt

SEQ ID NO: 14 - DNA, 200 bp, unidentified
  source 1..200: mol_type = unassigned DNA
atttgttttccacctccctattcagcaacccatctggtccttatgcaaagcacttgccttctcccgggatggggacacacatcagagcacagcgtggtgttttcccaagcccccgaggcacctgttccctcacattacaccttgaggagagtccagtgacctctgctgcagaggcgacaaaggctccctgggaagccggc

SEQ ID NO: 15 - DNA, 200 bp, unidentified
  source 1..200: mol_type = unassigned DNA
ggcagagaacgcgcgaaagggcagggccttggccgggaagtaccgcccagcgaaaggctggcaagggtgcgcagctggagcgtggcctcgggtacccctttccaggcagccgaactccccgtatcccagctctgcttggctgactcccatttgtcttgagggaggatcccctagtaggactgaatcagaagtgcgcccgt

SEQ ID NO: 16 - DNA, 200 bp, unidentified
  source 1..200: mol_type = unassigned DNA
cctttgggaggcgtttgacagctcagaaagcatggcctttactcaggctcagagacacacaaaaacctttcacgttttcctttcagcttcgcagccccgtccttctccacctgcacgggcacccactgaagcacaggacccttctgccaagccatgagccttctcaggcccctgccgcccgcctcgcctgaggctgcgct

SEQ ID NO: 17 - DNA, 200 bp, unidentified
  source 1..200: mol_type = unassigned DNA
atcccaacctccaggagaaagcatcatttcaccttttgttctccaggcttcactgtggcctctccatccatctcagtccagaaacaagatccaggtggacttttcttctccttatgagatgcaggtgtttggacgacttctagttgctctcctttaaacctggagactgagaagtcaaggtggtattttggaaagcagct

SEQ ID NO: 18 - DNA, 200 bp, unidentified
  source 1..200: mol_type = unassigned DNA
agttagattaaccttctcaactgactttactctcaggttttaaaggcatccacatgatatggtctagttaaaaggaggaagagagcagcaaactggaaaaaaagaataaaatattttcgtaggttctgtacagatctggtagtattatctgcgtgtctggttcactgtggtaggcgccacagaaatgggtagattgataa

SEQ ID NO: 19 - DNA, 200 bp, unidentified
  source 1..200: mol_type = unassigned DNA
cacctgccaaacagtgtcaagggtgaaaggcaaagatcgaaggccaaagtgcccctagcgtcccctgcagcaggtctcagtcaaccttaatagatctattaatagaaagaggctggactgcgcaagggcagggggactagcctggctaaaagcaaaataaaacacatcaacagacacactcttcttttgttaggaggagg

SEQ ID NO: 20 - DNA, 200 bp, unidentified
  source 1..200: mol_type = unassigned DNA
agcatgcacttgaagatggagttcagatatccaggacgctccaggctttgacccgcgatggtgagcaagggttcctcttccttttcctatgctgggcttctcagtcagtttcatcacaaaaggtgaaaggtagctagtagtgacacattccagcaccagaagaatttctagaaattattgtctaagggtctttaatttcc

SEQ ID NO: 21 - DNA, 200 bp, unidentified
  source 1..200: mol_type = unassigned DNA
ggaaaaagggcaggaggggtctatgaagcagtcctggggaggacagattcgcagtgtttgaatgtgggaagaactaatcagtgaaacattttttaaagccaagtttatcttcgatctcctgcagtttctgaaaggatagttcaaaattccatcaagtagttctgacttcctgctacagaaatgcaatcgcattgacaata

SEQ ID NO: 22 - DNA, 200 bp, unidentified
  source 1..200: mol_type = unassigned DNA
gaatcttttccaggaaaaaaaagaaagaaagaaagaaagtaaatcccacccccaagcgtgcagtctgcccacaggaagtgattcatggtgctccgattcgggcagacctgactgcaggaggcgggaggctgggaaccaggtctgggaaaagagggcagagagaggatacccagacctaatggcttctgctctgtggaaga

SEQ ID NO: 23 - DNA, 200 bp, unidentified
  source 1..200: mol_type = unassigned DNA
gactaataatttgaaactacaagctaaccagatatagctttgcagaaaccaaagccaacaacaacaagaagtatgagctgttgacgtcagccctgaaagtgttgaaaataacagtaggcagagaaacacctgctcatgtgattgcctcacaaggatcttgttgcctgcaatttagtcagtgccaaatgggtgattattct

SEQ ID NO: 24 - DNA, 200 bp, unidentified
  source 1..200: mol_type = unassigned DNA
gcgggcattgtgaggattcagagagaatccagagtttgtcctcagcacagggcccggcgggctggtgtgggggccagtggccgtcctgggtaagctctgtgatcattgcaggtcctccctggcaggtgagacccgctctgctgggcagtgtggctgacaagaggaggctctcggggagcgctctttgtccctatgctgtg

SEQ ID NO: 25 - DNA, 200 bp, unidentified
  source 1..200: mol_type = unassigned DNA
gaatcccgcgcggtggatgcaaggtgggattccagcccagcgacgcgccgcgtggccgggagtgcagacaggtcaccggtccccgggtgtctggaccgcgccgcttcctactgggcttacttcgggatctagacttcggcctccgtccttgcaagggcccagatttgattccggttcgtcgaggcggtctgtagcagata

SEQ ID NO: 26 - DNA, 200 bp, unidentified
  source 1..200: mol_type = unassigned DNA
ctgatacaaaagctccacttgccggatccgggcgagcattgctgcttccaccgggatgcgggaaaaacagtgcagagagatcctgagcttccgcacgtcatcatttctcagagtcgccatctgttccttcaagcaagtaacacctttggggctgcagtgtggctcagagcaggtgtctggcctcgtgtgagataccccag

SEQ ID NO: 27 - DNA, 200 bp, unidentified
  source 1..200: mol_type = unassigned DNA
tcccgttcccggtgttggggccaaagctctgcttgatggggtctcaagttgcctttcttccccctccccccgcccgcagcagtccggatgccgcccgcatccgagtcaagcgctaccgccagagcatgaacaacttccagggcctccggagctttggctgccgcttcgggacgtgcacggtgcagaagctggcacaccag

SEQ ID NO: 28 - DNA, 200 bp, unidentified
  source 1..200: mol_type = unassigned DNA
tactcccatgcagattcagcatcccttttctataacagagcaaaccgagctttactgggttagctccacatttgactgcagacactgtcccttaagctagatttttaccaaccacaataatggcatctatttctcttgcatcatttccattcaacaaatatacatattttcaatggcctaaaactccaagtatgcaaagc

SEQ ID NO: 29 - DNA, 200 bp, unidentified
  source 1..200: mol_type = unassigned DNA
ccatgcctctttgctgtttgtggaaaataaagcattctataggcggagctagtgaacgcctcttttaaaacacgagtctccacacttccctgttcactttggttccagcatcctgtccagcaaagaagcaatcagccaaaatgatacctggaggcttatctgaggccaaacccgccactccagaaatccaggagattgtt

SEQ ID NO: 30 - DNA, 200 bp, unidentified
  source 1..200: mol_type = unassigned DNA
cgccaaagtattttgagtccctgaggtcaggccagagcagcctcgtgcccccactacctgagaagtgcccccggcctcccaggggtgctgtgctgggcaaccactcatccctggagcccctcgtatgccctcgctgccctccgagtgggctagccacccctgcagctgccggggagccagctgacctccggggcagcctc

SEQ ID NO: 31 - DNA, 200 bp, unidentified
  source 1..200: mol_type = unassigned DNA
caaccagactggcccctttactccttcctccctcaacctgtccatatgctattatgtttgcctacacatacaggttcactgttggaaatctgtgagtgggacatttttctgaatttttattttagattcagggggtacatatgcaggcttgttacctgggtatattgtgtgatgctgaggcttggggtacaaatgatccc

SEQ ID NO: 32 - DNA, 200 bp, unidentified
  source 1..200: mol_type = unassigned DNA
cacgcatctctctggcctgttggctcacatttgggggtgtggcatttgtgcgtgaagtggagcctggtccctggtgacccaggtacagtttacgtctggagccagcatttggaaagttgcctccagggccctggatttggagggagccaactgccagagtctcaagcaggtgagggtggggtgcaggagaggagcgttca

SEQ ID NO: 33 - DNA, 200 bp, unidentified
  source 1..200: mol_type = unassigned DNA
agatatccaactttaaataagggacaagaaaatcgatacctatgagatgctcactgttttcaagtaatagtaatcaatctgtttttgaatcctaccaggctgtctacactgggtgcatggattagttagttattgatgcataaaaaccacaaaaaacttagtagctcatgattgacatgctcagccatttaggcttggct

SEQ ID NO: 34 - DNA, 200 bp, unidentified
  source 1..200: mol_type = unassigned DNA
ttactccagctgaggcatcagatgtgtgggtgaagaaaccaccagacgcggtggctcacgcctctaataccagcactttgggaggccgaggcgggcggatcacgaggtcaggagtttgagaccagcctggccaacatgctgaaaccccgtctctactaaaagtacaaaaaagaaaaaaaaaatagccgggcgtggtggcg

SEQ ID NO: 35 - DNA, 200 bp, unidentified
  source 1..200: mol_type = unassigned DNA
gctcccagccgccgtgatatatcagcttatgtcatcctgacacctacgcagatgtcggctcgaatccactttgcctgagcattgtctcagagaaatctaatttaaaaattaggcagcaaatagaaaatatatttgactgctagagatgcaatgggactgggagcccaacaaaggatcttaggcaaaagaaatccaagttg

SEQ ID NO: 36 - DNA, 200 bp, unidentified
  source 1..200: mol_type = unassigned DNA
ctcgacatgattgtcccctgtcggccctgcgccgccgaggccgagtgacaaggcgcaggaaccgcctattcggtggggctaccagcggaaggaagtttactctgcagtggacactgttttagcactttaagagtagcaggaccattcctaaataattgaggttccccctcctccacacaaaattttaaaatttcacggat

SEQ ID NO: 37 - DNA, 200 bp, unidentified
  source 1..200: mol_type = unassigned DNA
ggttaacccagattgccttcggtgcctgagggacttgatattttctcattatgaagactttcccatctagtgaacgctgcaaataaaactgctcagaatcggtctgaactgcctgtttccgtggtagcaggcctctgtctggaggataaatctgcaccatcttcttttttttttttttttttttgggtttcagccagttg

SEQ ID NO: 38 - DNA, 200 bp, unidentified
  source 1..200: mol_type = unassigned DNA
tgggctaaaaaggatagacaaggaacacatcctgggccggtaattacgcaaagcattatctcctcttacctccttgcagatttttttttctctttcagtacgtgtcctaagatttctgtgccacccttggagttcactcacctaaacctgaaactaataaagcttggttcttttctccgacacgcaaaggaagcgctaag

SEQ ID NO: 39 - DNA, 200 bp, unidentified
  source 1..200: mol_type = unassigned DNA
tgttgagtgcaccctcttgttataagagctgatatcccgaagtatacatgtgcaaggaacatgcctgcttcttcagtgtgcattccaacatgccacagaaatgtggtttacatcatagcttctaccgccgaatagtctatctttagattttgtttacatggtttgtctcaattagcttaatacataattagctattgttg

SEQ ID NO: 40 - DNA, 200 bp, unidentified
  source 1..200: mol_type = unassigned DNA
ggaggattccttgagcctaggagtttgaggctgcagtgagctatgattgcaccactgcactccagcttgggtaacagagcaagaacctatcttttttttttctaaaaaaaaaaattggaatatggaaagttgatcggataaacgttgttttatctttgttaaaagataaatggtaaatattaaatttcccaaagttggta

SEQ ID NO: 41 - DNA, 200 bp, unidentified
  source 1..200: mol_type = unassigned DNA
gcaggtatagtcccatgaccaccccccgagcatggggttatctcattcttttttttgggggcgggggggacggagtctctctctgtcgcccaggctggagtgcagcagcgcgatctcggctcactgcaacctccgcctcccaggttcaagcgactctcctgcctcagcctcctgagtagctgggattacaggtaggcacc

SEQ ID NO: 42 - DNA, 200 bp, unidentified
  source 1..200: mol_type = unassigned DNA
ttggctctcccaagttattcttggtccacacttggccagcactaacagagcacacgtggcagtattcacaaatattagacatattttaattggaggtggcaacaggggagaaatatttttggttccttctcacctgtgcctccttaacctgcccaggggactgtgaaaagaaaaagattctagaagcacactttagaaga

SEQ ID NO: 43 - DNA, 200 bp, unidentified
  source 1..200: mol_type = unassigned DNA
gtctattatttttatgcctttgggtcctcatatgcatctgctattttcaagtgcctttagctccaaataatccttatgccaaaatggcatatttgggggcaacacattctgccacccttcagtacctttggccaaacagaggtttgctttccagacctcctgggttctgtgactggcaactgcacaagcagggcacgctc

SEQ ID NO: 44 - DNA, 200 bp, unidentified
  source 1..200: mol_type = unassigned DNA
gtgggtggcccgtgtacaggaatgggatcgcacggtggtccagatcgtctgtgcacccatccttgccctgcctccctccttctagggggttggctggaactcgggccccgctggccggcggtaggagctggaatgcccgagctctgagaagctcttcatctatcagcagtcagctttccacttaagcagagccaagttta

SEQ ID NO: 45 - DNA, 200 bp, unidentified
  source 1..200: mol_type = unassigned DNA
agctcgcctgggctctgtagcttctctcagtagaaaggattcttccatggctggggtgagcgggctgggacacctgcaggaaaggctcctcaggaagaaataactggaccttcaataacctttttttgcagtctcaatattggccttagcttggagtcactgggctcaggcttcaattcccagtcagagttattcacttg

SEQ ID NO: 46 - DNA, 200 bp, unidentified
  source 1..200: mol_type = unassigned DNA
gtggatgagagggtggggccctctacggagatgagatcttacctatgtaaaaccacgctccagagttcctaaaccacaaccctacctgggaagcacccagggctgagagttggtctttaaaacattcttagcctataacctcttctttaaaaatctgttgtctcttctagaacaaattattttaaaccagttcaagaaaa

SEQ ID NO: 47 - DNA, 200 bp, unidentified
  source 1..200: mol_type = unassigned DNA
ggctcttcccatcgacaactgggcatagctgttggttttgtgccaatcaccccacttgggaaaccctgagccaccatgcctcagggaaagtggtggcacttcctggtggcaccaggaatcgcacaaggccatcactgccgcctggtgcacagggctcccgcggctggcatctccagagcctggaggaagagggtagagga

SEQ ID NO: 48 - DNA, 200 bp, unidentified
  source 1..200: mol_type = unassigned DNA
ctacacctgagaacgacattcactctgcttcattgaaaacaatctatcccgtgtgtggaagaagcatgccttgctggggtcagggccagggacaaagctcagaggccatgctgagttttcatcaaacacctgctgagcagctggcacgtgccaggacacggtctaggctgaaggctgccatcgtagtgcagaatagaatc

SEQ ID NO: 49 - DNA, 200 bp, unidentified
  source 1..200: mol_type = unassigned DNA
actcatacccctcctcctcctccgcgttattaatccagcagttccctcctcctctgggtctgatgctgcagccctggagtattgcagtatgtgtgtccactcctgctgcctgcccaggattggtttgggcgaagtaaaactgggagttttaagtataaacggcaacaacagctctttgagagcattttagaaaagtgaaa

SEQ ID NO: 50 - DNA, 200 bp, unidentified
  source 1..200: mol_type = unassigned DNA
ccggggcttggggggcggccgggcaggggccactcggtgctgcgggggctgctgccctgcggggcatcgctctttctcggccggggcatcccccgacggggcgggcggcggcggcggcgggggctggaacacccgggaccgcttgctgaccagcttcgagttgacctggggaacccctgcggccctggggagcccgggcc

SEQ ID NO: 51 - DNA, 200 bp, unidentified
  source 1..200: mol_type = unassigned DNA
tgacagatttttattataatagtcatctccttctaaaatctggtgagcagcttccctgccgactcaataatgcctattgctgaacatctgcagttacacaaaggactattactgttggctgaaagctttcttatagaggatacttctttagaaaaggaagtagcagtctttacctgcagttgggtggggatacactacag

SEQ ID NO: 52 - DNA, 200 bp, unidentified
  source 1..200: mol_type = unassigned DNA
tctcttcctgcccagcgtccacttactatggtctttagcccatggctggcattcagtaagtactggggggtgacagagtaagagagaacaggtgagtgggaatgcatcagtgaatatgtgtgaaagagaaagttcatgaaagcagaaggaaatagaatgaaagaggaagcgaagggggtaagcaaatggaaaagagagac

SEQ ID NO: 53 - DNA, 200 bp, unidentified
  source 1..200: mol_type = unassigned DNA
atacgtcaaggactctgaagccgtgagagagggggaagaacaacagtagagaggatgcccagctggtaagaatcgagtgtttatgaagttttagtcaattgatgaatctcattggctaaaatcaagaaacgctccgcctctttgcaaatatgtatgaaggagagaagtgcctaaacttctatgtctgatagcatttgacc

SEQ ID NO: 54 - DNA, 200 bp, unidentified
  source 1..200: mol_type = unassigned DNA
acttttaatacttaaataacgacaataataataatatacaaggttagttccaaggcatgtagaggtgatggccagcagaggtgaagccaatccaccctttctgggctaggggaagcccagatggtcttccgctcgctcggggtgaggcactccccagggtcccggcctggctgcccatcccccaccaagtctcccaggcc

SEQ ID NO: 55 - DNA, 200 bp, unidentified
  source 1..200: mol_type = unassigned DNA
ggttgacaagtaaggttgccaacggaagccagttgctctcttctgatgctggaacttacaacctgtcatcttttgttgtctgcagagcagagaacatagccaagccggcagggacagtctgttgtatgagtctggatctcattgcaatgatgcaatttatttacacataccaaggatgagccataaatccttaccactgc

SEQ ID NO: 56 - DNA, 200 bp, unidentified
  source 1..200: mol_type = unassigned DNA
ttggggcccagggacacatcttccctgtctttctctatctccctcctcccttcttaccccaggcttgggaagtctgagtgtttctggaagtctgctgctgtgtgggaatcactcttgtcttgctgcttaagacacgtgggagctgttaatggctcacatttcaaagcctggctttggggtaggatttaatgcaagccacc

SEQ ID NO: 57 - DNA, 200 bp, unidentified
  source 1..200: mol_type = unassigned DNA
aaaactcatgactctgactggttaaaatgagtgtcagtgttctccatctgtcctcgtaacagcatcactggctctatattgtcagatctttaatactaactttctgcccagtgagcaatgactcatacaaagctcagtgcccattggttcttttctcagagtctgtccaatcctagggtcacagaagactgcttgggttc

SEQ ID NO: 58 - DNA, 200 bp, unidentified
  source 1..200: mol_type = unassigned DNA
aaatcaactccagctccctctgcgaggctggcatgattgttccatttcacccagccactcagtcccttgcctgttacactgtggggctgaaacctaggcaggccgagccccagccaccccagctctgagccgcctccccacccctcacctgatggtccactgtgctcccgtagagcccgttgaggttggcgtagtggcag

SEQ ID NO: 59 - DNA, 200 bp, unidentified
  source 1..200: mol_type = unassigned DNA
aagaggggtgagagcctcgagatgatgggtagtctctgactcttaactggatctgcttcacacctaggtcccgacatctgtggccctggcaccaagaaggttcatgtcatcttcaactacaagggcaagaacgtgctgatcaacaaggacatccgttgcaaggtgtgcctgggggtggtggcaaatggctgtcatgggga

SEQ ID NO: 60 - DNA, 200 bp, unidentified
  source 1..200: mol_type = unassigned DNA
ctggagtttctgataatttccaaattgaacaacctcagtcctaatgagtcagaggcttgtgcaatattttcaaacctcaggaacatctttttcattagttgtgcaataaagatggtaggcctatctctgtgatgagctgtttttttttctcaaagtttgatgagattcgccgtagaattccttctcacatagtcttgggc

SEQ ID NO: 61 - DNA, 200 bp, unidentified
  source 1..200: mol_type = unassigned DNA
ggacacagccctcaatctttgccccttgcagatgatgtgccttttgagaggcaccaggtgactgctcacagtatgcaagcgcattatctaaaagcactccctcatttggaaacacttcaatgaaaaagtgaaattttgttcacaatccactcccattaatttcagaattttcaaggctgggcaccgtggctcacccctgt

SEQ ID NO: 62 - DNA, 200 bp, unidentified
  source 1..200: mol_type = unassigned DNA
ctgtatagcatctttctccacctaccaaatggggggccgtgccatctctgcctctcggtgaggagatattacgacacagccagtgagattcacaaagcaccttgcagagtgggcctactctacactctggttttcagtttttattggagtcacaggtgggaatgcatcaaacaaaagatataggctctggaatgtacggg

SEQ ID NO: 63 - DNA, 200 bp, unidentified
  source 1..200: mol_type = unassigned DNA
tgcacacatcagcaagagacaggaaaggtcatctcagagccgggagtcacagctttctcctccatctgtccacaccacctccctcctccttcatccgtccaaaccatgtttctcctgctgcatccgtccacaccacctccctcctcctgcatccgtccacaccaccttcctcctgctgcatccgtccacaccacgtccct

SEQ ID NO: 64 - DNA, 200 bp, unidentified
  source 1..200: mol_type = unassigned DNA
gctggccaagttgagcgaggacattgtcttttgttgggaaaggatactttttaagtgttgtttctaaattttactacatttgctgaaaagacaggacaggaagcttttaagaatgaacaatctcccagcctgtgtgggttggtttgagctgtgagaggtgcagctgggcttccaagtaggcttatatttccagactccca

SEQ ID NO: 65 - DNA, 200 bp, unidentified
  source 1..200: mol_type = unassigned DNA
gtgaaattcgagattgcgtatttcaccaacacaggagcacagccctcgggaaactcagcctagtcaggcagtagagagttgtcccggagagaagtgatcctgcagactcgagaaggggcatgatgatagcacacgtctgttgagcacccagtctgtgtgccgggtgtgttacctctgtgacctcatttggtcaaacgagg

SEQ ID NO: 66 - DNA, 200 bp, unidentified
  source 1..200: mol_type = unassigned DNA
cgcagagctggccggggcattcgtgcgtgttcacgaccgagaacttttcacagagagaggacttctgtcctgatagacgtcggtgaggactgaatccccacttacaggcgtgcacatcagggactgatggacatcgctgaggaccgatcccagcttacaggcgtgcacactggaggctcggaagagctgactgtgactca

SEQ ID NO: 67 - DNA, 200 bp, unidentified
  source 1..200: mol_type = unassigned DNA
cccgaggactgggatgaagagatggacggagagtgggaacccccagtgattcagaaccctgagtacaaggtgagtttggggctctgagcagggctggggctcacagtggggagtgcaccaaccttactcacccttcggtttccttctcccttctgcagggtgagtggaagccccggcagatcgacaacccagattacaag

SEQ ID NO: 68 - DNA, 200 bp, unidentified
  source 1..200: mol_type = unassigned DNA
gtggataagtttttctccacgtgggccacattcttactagaaaagaaaccccggccctttagctcatcaccctctgctctggctttccccgagttggtacagagctgatgccgttggtatttttaagaaataatcttgagtcagaaggaatgtgccaaaccggcacaggtgagagatctctggacccccttatatttgcc

SEQ ID NO: 69 - DNA, 200 bp, unidentified
  source 1..200: mol_type = unassigned DNA
catctgctttctgcctgacagagccaggcttcctcgggacagccagacaggcggctattgttcaaaagtcagggccggtttgatgggctctcccgcccccagtagatgagctaggaagatgtcacttttaattacaacggtgagggaataggtataggtgtgtgcgtgtgtgtgtatggagcagggttccacaggcttgt

SEQ ID NO: 70 - DNA, 200 bp, unidentified
  source 1..200: mol_type = unassigned DNA
acagtcagagcacggcctggctgctcatgggctggagaggttttcctgcccagttgcctgagtcacgctcttcccccaggcccgcccttcctgcccttcacatctggttcagggctctgccgaggccttctggggcaggtgccaattatcgctcaggaactgctgcttgcttcccagggctggctttctgtgaagggacg

SEQ ID NO: 71 - DNA, 200 bp, unidentified
  source 1..200: mol_type = unassigned DNA
aaacctccagatggcaccagagaactgagaaaaacaattccattgccaaggagcctttgtgttgctgacaagagaggagcaggctgagagaaaacgccctgtttgtcacaaagttacatctgcgagctccttcaaggaagggaggtgcctgacgctccttgttaatgccggttgctgttacagacttgcaattgtgcaac

SEQ ID NO: 72 - DNA, 200 bp, unidentified
  source 1..200: mol_type = unassigned DNA
atgattatctgacacgatccaagaatccacagtgtaacaggctctatgactccattaaagaaaagtcagtttaattctgtgctatgctccagattacttctctcctttcctagatctcctctatcaaagagagtagacgtttataaaatggtgtctgaattcaactcagtgtcctggaagccatatgaaatatttttttc

SEQ ID NO: 73 - DNA, 200 bp, unidentified
  source 1..200: mol_type = unassigned DNA
caaaaacccaggggcgggaggcacatgcgcgttttgggatcagggtgcatcctgttggtggagtgtgacttatgctacaccagagggggtttggtgggaacagggcccagggcaaccactcagggcctcgtctgtcaggccgggagacccggactcttacctgtggagatgtggagctgggaatgacgctgtcccgaaaa

SEQ ID NO: 74 - DNA, 200 bp, unidentified
  source 1..200: mol_type = unassigned DNA
ctcagacaggaaaaacaggacccagatggctcagactgttgggttagcatctggccttcgacaggtcaccctggcccgccagatcagccccaggcctgacaaaagcactacaggataaagaataggatctcattaaatgcagtggaagtaacgtgagaaggatggcttttcatatgccatcctaaccctcccctcccccc

SEQ ID NO: 75 - DNA, 200 bp, unidentified
  source 1..200: mol_type = unassigned DNA
ggttgacacaagaacagatgcttgataaatgtttattggataaacgagcgaaggataatcacctgagcaccatagttgccaaagccccctctagtcccaaacctaagcccacactctgtccccttgcaagtaaagaaatgcaccaagcagggcttatgcagcagccctcggtctggcctggctggagctataaacatgca

SEQ ID NO: 76 - DNA, 200 bp, unidentified
  source 1..200: mol_type = unassigned DNA
tttgctatgagcaggtttaagttaatattacagaaaggattagcgcaattcctggcacagggtgcctgctcagtaaatatgagctattgttattttactgcgggttcccaatgagtgccccactgtcacgcacatgcatccccccggatcccacacccgacccagctccctctggatgggcacaccaccagagtcctgtc

SEQ ID NO: 77 - DNA, 200 bp, unidentified
  source 1..200: mol_type = unassigned DNA
agcccttgaagggactagaagagtaaaaaccctagaaaggactagagagatgccccatccgcctcatacctgacatataagggtcactgagaggcatcccaccaaccccctacctggcctatagtcatctgtgaggtggcagccaaagttgtagatgagatcgaggtgacgtcgctcctgtaacctgatgcccacatctc

SEQ ID NO: 78 - DNA, 200 bp, unidentified
  source 1..200: mol_type = unassigned DNA
ctctctcccactgcaaacacctggacaagaacacagccactggcaggcccagcctggctggtagggagaatacatgctgaggatagatgcggaggtatgggtgtgtatgaggtgcaacctgccaggacgaactgttgcatcacagggtgagtcatgttgaggaacagcggcagtagcaggaccacatcctttctgggatc

SEQ ID NO: 79 - DNA, 200 bp, unidentified
  source 1..200: mol_type = unassigned DNA
caggcgtccctgaggcttccagctgaatctttctgtccctcctaggcgtggctgtgggaatccttggggcgcctttgggttccaggaagctggggctgtgtgggccgggctgctaactggggctgtggagcaatctgccagccgagccccttccggagctgtgggttcctctgggtgtggggaagctgccctccacaacc

SEQ ID NO: 80 - DNA, 200 bp, unidentified
  source 1..200: mol_type = unassigned DNA
ctttggttagctgcactgtgctgagcgcagctctgcagcgaggtttagggagctgcggtgcgttaggcacaaggcacgtggcgcgcctcgctccagcactttgcaaacgcacagcagcgcagggccagagcggctggcccccaggggtgggggcgaccagggacaggaggtcccatatggtgtcgtcgcgttaaaaacgt

SEQ ID NO: 81 - DNA, 200 bp, unidentified
  source 1..200: mol_type = unassigned DNA
gaggccccagctgcactttcagtcacgtgacctggtgactcaggccctttctggttcctgcttgtcagggtatgggaaaggtgagaaactactttgcagtggctctttgggagggtctagggatgagcaatagtttgttattatacaggttgccctgaaccctttttatatgagcataataaagttatcttataacccca

SEQ ID NO: 82 - DNA, 200 bp, unidentified
  source 1..200: mol_type = unassigned DNA
aatgtggtgctaagtctaaagttttaacgcattaaaggacagcatctgggggagaaaggccaacgctttatattgtcatgactgtggtgaaacttttgctttccaagtggaagctgttccggttgtgcaaagccggatggcctaggctgctgtctttcaaaggtggagagcatgtgcgggtggggccaggccgggtgggt

SEQ ID NO: 83 - DNA, 200 bp, unidentified
  source 1..200: mol_type = unassigned DNA
atggctccgcaggtgaattttcagccgacaggctttatcatactgcttgctgcacccaggaaaggagcaggaaaagagctcttgttccctgaagtgggctcggttatgggaaaacagggcactcactgtgataaatgtcttctcacagcctgaacaaggaaaacacaaaccctgctcaaagcgtggcaaaaattatacaa

SEQ ID NO: 84 - DNA, 200 bp, unidentified
  source 1..200: mol_type = unassigned DNA
tcgaggacagttccgtgtagaagtggaggcacacagtgaaggctttgagaggcttcgttaacggtgctttgagggatacataggaagtatccgactctttgggaaacacaaaagccttcctcgacatgtctgtgagccagaaaaacaagcaaatgtgagaggtttctcagatcacacagatctatcccctcacttacgta

SEQ ID NO: 85 - DNA, 200 bp, unidentified
  source 1..200: mol_type = unassigned DNA
ttattattattttttgctttactttcacagattggtggtaatgagtgattgcttaaagcaatatacatccactttttttgttgttggtttattggttttgttaaaactgtctccaaagttttcactgaaacattttgaatcacatcaatactgtgacgtgtactgtgaatagaagagacggccaggttttggccagcact

SEQ ID NO: 86 - DNA, 200 bp, unidentified
  source 1..200: mol_type = unassigned DNA
ctctaaccatatgtctgtgtccacacctcgtgacaggaaaccagggtgacagaggagacaaaggcgcggcaggagcagggctggacgggcctgaaggagaccaggggccccaaggtacgagtccacggccagcaaggcttcactgggtgacatctcttccgccatcttgtagctttatgtggggcgtgcttgggttatga

SEQ ID NO: 87 - DNA, 200 bp, unidentified
  source 1..200: mol_type = unassigned DNA
cctggaaacactcacgctcaaggagcggaggtgaatggcttcctgttctcacctccgggtccaggcactgggggtgaggaagtggctgggaggccaggaggctggaggaggggaggtgctggggcccagggctgtgcgggaggggatgggatcctgtgggtaccttggcccgtgtgccagggtcgggggaacgtgacact

SEQ ID NO: 88 - DNA, 200 bp, unidentified
  source 1..200: mol_type = unassigned DNA
ctgacccgtggttcttgaacaatttcagtgggcacgtctggctttctttgtcgagtggaggctaagcttgcacgagttaattttgttttcttccgtgatttttttttgaaagccgcagggctgcacttggcgagtgctagaaagacggccacgtttgggatcgtgggccggatggggcctccgtcggccaggcccagttg

SEQ ID NO: 89 - DNA, 200 bp, unidentified
  source 1..200: mol_type = unassigned DNA
tctccatcatcgcacctctgatcctagagaagcccagtctgttctcttgctcaatcctggcctgagtccctggcatcagcatcctctgggtcccagtcattcggctgctcagctgtggcctcgctcgcctcactccactcggaaggagggaagggggtgccagagaatagaacttcttggccctaataggacgggagaag

SEQ ID NO: 90 - DNA, 200 bp, unidentified
  source 1..200: mol_type = unassigned DNA
tgaaagcaaaagagcccggagacgctcccgtgggggctggtgtgcgtgtctgcagaggctgccgtgtgagcctctgtgcagagcttggctgcacctcagcttcccgggctgcagagtgggcacacaggacacccatttctccgagtgtcctgttacgggtgcctcctcccgttcttttttttttttttttttttgagctg

SEQ ID NO: 91 - DNA, 200 bp, unidentified
  source 1..200: mol_type = unassigned DNA
gccgagactatctcaggtggccttgaaccactgttggcaaagggacagataacgagcccagggcagtgtgggggactttgcgttttgaagtctgggtcagccagatagtaagcatcttttgcttttcctgctataacagatatgtggccatcctccctcagttgtatatgcagagcctaagtttgcacagtgtcctcctg

SEQ ID NO: 92 - DNA, 200 bp, unidentified
  source 1..200: mol_type = unassigned DNA
gatttgagatgggtaaggcctccatgaatttcaacaatgcatttaaaaaaacaagcttttgttaaacttggttaaatcatatacttacaaagccacatacaagaacactagcttgacttagcacagtctactcagcccagaacctgtgagctgtgtagctactaccccccggtggtgggcaaagataagttcgagacaag

SEQ ID NO: 93 - DNA, 200 bp, unidentified
  source 1..200: mol_type = unassigned DNA
acagtcaaatactggggacagggaagtttcatctagaaaggtaggggtgtaaggggcacctgcaccccctggggcagcatgctaactcacctatttgtgtcttcaagaccccagaaatcacagctggtaggtggtcaagcagcagtgctctggggcgattctgaggtaaggattgcaggcagaaccagggaggaagttaa

SEQ ID NO: 94 - DNA, 200 bp, unidentified
  source 1..200: mol_type = unassigned DNA
tgactcaagccataaactcattctcttcatccagcaaacaagagcagaaagtctttcacaatcttttttttacaatttactttttaaccgcccttaatccagttattccttaaactgccgccttctatttactaatttctttaaaaaaaaaaaaagagagaaagaaaaatatatacatttgctgcttctatatcattggc

SEQ ID NO: 95 - DNA, 200 bp, unidentified
  source 1..200: mol_type = unassigned DNA
tctgctccccctcccccacaacccccaggagaccccatgttggagaagcgacccactgggccccgccccgacccccagcattgttttcggccctacggaggagcctcattaggaagaggacgcaaaagggaatcgctggcccccagtcttgaaaagcacttccttccccaagggcttggtacccgttgggtgggtagaat

SEQ ID NO: 96 - DNA, 200 bp, unidentified
  source 1..200: mol_type = unassigned DNA
gtgaggggcgggggtgaaaataattggtcagttttatgaagggctatcaagttggcaatgagtagcatagaccaaatgtcaactttctgatctgatttcccaaacgtacaccctcaaaaccacaaggtaaataaataatgccttgagtcagcaaaaggggacaaaacatattttgagaaaaacatttgaaagaatattaa

SEQ ID NO: 97 - DNA, 200 bp, unidentified
  source 1..200: mol_type = unassigned DNA
cgcctgtgaggtgcttatcacctgatactgcctcttcttgctggcacatcaagctggaggattgcagcatctgaacacgaatgaccaatcaaatctcgggcctataccccacagacgtaacctttatctcccaccagagggcaccagtaacctataaaaatctaagggggcgggcggtccctcttgcccttaacatcgtt

SEQ ID NO: 98 - DNA, 200 bp, unidentified
  source 1..200: mol_type = unassigned DNA
atctatcttcctaaaaagcgatggaaaaaataacggctttcaagagcaatagtaaattctttttttttttaatggagttgtctatcggggttactgataattgtcaactgaacccttttgaaacacaggtttaaataaaccttttcataaacatgtccactctctccctcagtccctcctaccctctgatcctgttcttc

SEQ ID NO: 99 - DNA, 200 bp, unidentified
  source 1..200: mol_type = unassigned DNA
ttggcactgtcagtctttttcatgttagtttagggtttattgcattgttagagctgttggggtagggaagcttacaacaacgagaatcattgaagaaacgtgagtgatgctcaccttgaccttgctcagggtaatcggcttaggaaattggctttgagtatttcttgtcctccaggcaggagagaggctctgccagcagg

SEQ ID NO: 100 - DNA, 200 bp, unidentified
  source 1..200: mol_type = unassigned DNA
gaatggcgccagagtgattcctaaaacccaaatgttattttgcctctactgcttaaactcctgtggccgttccctatcttccccagatgaaaaagctcaatatccttagaaaggctggaaggaccagcccagctggtgctgcctggcttccatctccccgtgagaaaacggagagctgtctggtacaagagcttgcgagg

SEQ ID NO: 101 - DNA, 100 bp, unidentified
  source 1..100: mol_type = unassigned DNA
caagcagctgggagcacaggcacgcgacaccaagtccagcaaatatttttgtatttttagtagagacggggtctcaccgtgttggccaggcttgtctcta

SEQ ID NO: 102 - DNA, 100 bp, unidentified
  source 1..100: mol_type = unassigned DNA
ttcgttcatgtgacagtattcttatttcagtcctgccatgagcaggcacagtgagtgcccggtctcctctctgctcttgcttatgggaaggggatgctgg

SEQ ID NO: 103 - DNA, 100 bp, unidentified
  source 1..100: mol_type = unassigned DNA
ctcaatttccctacctgtaccaagctgagagcgaactaaatgtccctgaaggttccctgtgatccaccagcgacagctccctccttataagtacatgaat

SEQ ID NO: 104 - DNA, 100 bp, unidentified
  source 1..100: mol_type = unassigned DNA
ttccaaaaccgaagaacttggggttcgatatttgagggcaggaaacatccagcacgggaaaaagatgtaggctgggaggctaggccagtctcgctttttc

SEQ ID NO: 105 - DNA, 100 bp, unidentified
  source 1..100: mol_type = unassigned DNA
gcacagatggacgtccgtggaagaaacgacccacccatagtccagcccaaacccagggtgagcgcagagtctacacgctggggactcctgggacgggctg

SEQ ID NO: 106 - DNA, 100 bp, unidentified
  source 1..100: mol_type = unassigned DNA
gcaaaaacgcgtggccaggggaaggtgtgatcggtctgagggctgatgggggagccaaggagaagggaatgtgcccagcgcacggatgtggtcagggcgg

SEQ ID NO: 107 - DNA, 100 bp, unidentified
  source 1..100: mol_type = unassigned DNA
gaggcgcgtccgcaccgattttctggaccactagggagatgagcccgtcgtcaatgtagatgcggccccccaccggcacgacccggacaatattggggta

SEQ ID NO: 108 - DNA, 100 bp, unidentified
  source 1..100: mol_type = unassigned DNA
ggccctggcccagcgagtcccagccccactgctcaccccctgcaggatcccagtgcggatctccggtcccttggtgtccagggcgatggccacgggccgg

SEQ ID NO: 109 - DNA, 100 bp, unidentified
  source 1..100: mol_type = unassigned DNA
gctcgcgggagtacctccagccctcgcgggggtacctccagccctcgcgggggtagcagcacgagctggcggccgtcagctaaggcggggaagacaagag

SEQ ID NO: 110 - DNA, 100 bp, unidentified
  source 1..100: mol_type = unassigned DNA
aggggagggaggcgcgtctccctctggcccagctcccgccgtggcgccaggaaccttttgcatgtcctgggtggtgtaatggctccgcggggctgcgtct

SEQ ID NO: 111 - DNA, 100 bp, unidentified
  source 1..100: mol_type = unassigned DNA
gccccctgcgcctctccctaagctgggagctgagcccccaccttcatccccgcccgagaggagagagggctgaccgtgggcagagggggcctctcatatt

SEQ ID NO: 112 - DNA, 100 bp, unidentified
  source 1..100: mol_type = unassigned DNA
atttcaggggccgtaaggcggggtcgggtgacagcgcggcttcccgccccgtcgcagctgcccccaactaggcccagctcagtgagggagagtgaggcgg

SEQ ID NO: 113 - DNA, 100 bp, unidentified
  source 1..100: mol_type = unassigned DNA
ccgggccaaagactgagtgaccgggtgggggctgtcccctgccccactctccagcccatgcgtccctgcggtggcctcagacccttcaccccgcccgacc

SEQ ID NO: 114 - DNA, 100 bp, unidentified
  source 1..100: mol_type = unassigned DNA
ggtttggcccacccagctagagctccagctcggcccttcctaagccccaagttgcatagctgcctggtgggtgcgtgttccacaggtacctcggactcgc

SEQ ID NO: 115 - DNA, 100 bp, unidentified
  source 1..100: mol_type = unassigned DNA
tagtgaagcccaaggctcaggggttcaactgggaagcaccgacctgctctgggaggagagggctatctcactgtggtggtgtaggacctggcagaatgaa

SEQ ID NO: 116 - DNA, 100 bp, unidentified
  source 1..100: mol_type = unassigned DNA
gaatggaatggaatggaatggaaatgaatggaatggaatggaatggaatggaatggaatggaacggaatgcaatggaatggaacagaacggaatgaaccc

SEQ ID NO: 117 - DNA, 100 bp, unidentified
  source 1..100: mol_type = unassigned DNA
gcttgaacccaggaggcagaggttgcagtgagccgagatcacgccattgcactccagcctagacaacaagagtgaaactccatctcaaaaaaaaaaaaaa

SEQ ID NO: 118 - DNA, 100 bp, unidentified
  source 1..100: mol_type = unassigned DNA
gtcccggtctccggggatgccggcctcggcctgcctggtgggggcagcagagcagtgcggtcggtcggacgcctccctctccccgcttggggggcctcct

SEQ ID NO: 119 - DNA, 100 bp, unidentified
  source 1..100: mol_type = unassigned DNA
cgggcagccccaccctccttccgggcagcctgccctcccggccctgggcagggggggctcggggcgttgtctgatgtctgaggtggaggccgtcttcagg

SEQ ID NO: 120 - DNA, 100 bp, unidentified
  source 1..100: mol_type = unassigned DNA
tatatatatgtatatatgtatatacgtatatatctatacatatatacgtgtatacacgtatatacacgtatatagacgtatatagacgtatatatacgta

SEQ ID NO: 121 - DNA, 100 bp, unidentified
  source 1..100: mol_type = unassigned DNA
gtcctggtggggcgagggtcgtgagggcggcggccggcccgggaaaggcggccctagagggctccgcgcctggggcggggcaggggccgcggggcgtact

SEQ ID NO: 122 - DNA, 100 bp, unidentified
  source 1..100: mol_type = unassigned DNA
gcggcttgcgggtgtcggcgccgggcccggcctcagggagcgggaagcggtcgatggacaagtcggtgccgtcggcgaagaccctcggggccggagtctc

SEQ ID NO: 123 - DNA, 100 bp, unidentified
  source 1..100: mol_type = unassigned DNA
ccttggggctcaggagcagcgtcgcgcagccgcggatggagaggaggggaacgggctcccggctcgaggacgcgcgcagcggcttcttggcgctgcgggt

SEQ ID NO: 124 - DNA, 100 bp, unidentified
  source 1..100: mol_type = unassigned DNA
cgcctcgccccagacctggaggggcgtggagcggcgggtgaggggcggccgggccgggccgtcggggccgggctcccgggccggggcgcaccgtgacgtg

SEQ ID NO: 125 - DNA, 100 bp, unidentified
  source 1..100: mol_type = unassigned DNA
caggtgatcccctgcctcggcctccgaaagtgctgggattacaggcgtgagccaccatgcctggctggttttgaagaagccagccacatgagtttcacag

SEQ ID NO: 126 - DNA, 100 bp, unidentified
  source 1..100: mol_type = unassigned DNA
ccctgtagtcccagctactccggaggctgaggcaggagaatggcgtgaaccccagggggcggagcctgcagtgagccgagattgcgccactgcactccag

SEQ ID NO: 127 - DNA, 100 bp, unidentified
  source 1..100: mol_type = unassigned DNA
ccgtgccctgcggccttacctggctgtttcacagtgacttctgtgcgcccagaaacctcctctgccagcttgtaccaggcgtagttggggctcagcagcc

SEQ ID NO: 128 - DNA, 100 bp, unidentified
  source 1..100: mol_type = unassigned DNA
gttgagtgagacgcgctcctcccacgcggggaagagtgctgcctgatctggcgagcgcagggcccgtgctcctctctggcgggtgcgcgcgggccgtgtg

SEQ ID NO: 129 - DNA, 100 bp, unidentified
  source 1..100: mol_type = unassigned DNA
ctcacgcttgcaatcccaccacctccggacgccgaggcgggcggataacctgaagtgggaagtttgagaccagccttatgaacatggagaaaccccatct

SEQ ID NO: 130 - DNA, 100 bp, unidentified
  source 1..100: mol_type = unassigned DNA
tttcaacgtgttagccaggaatggtcttgatctcctgatctcgtgatccgcctgcctcggcctcccagagtgctgggattacaggcatgagccactgtgc

SEQ ID NO: 131 - DNA, 100 bp, unidentified
  source 1..100: mol_type = unassigned DNA
ctccctgacccctcaggcctgtcctgggggtccgccctagaaatagctcggaggaaggagccagtgacttgggtgacgttttggaaattcagtagagtct

SEQ ID NO: 132 - DNA, 100 bp, unidentified
  source 1..100: mol_type = unassigned DNA
actgtccggctccagccaggtccgctacgcctggagagcatggaggcaagttctgccggctccacctgcaaagcagagcccctttccacgactgtgcagc

SEQ ID NO: 133 - DNA, 100 bp, unidentified
  source 1..100: mol_type = unassigned DNA
gagactccatcacaaaaaaaaaaaaaaaaaaagagaaaggagacgaaaaacaagaagagctcagctttaaaaaaggatcacgacgtagaaaaagacacaa

SEQ ID NO: 134 - DNA, 100 bp, unidentified
  source 1..100: mol_type = unassigned DNA
tgtctctactcgaaatacaaaaactagccaggtgtggtggtgggtacccgtaatcccagctactggggaggctgaggcaggagaatcacttgaacccggg

SEQ ID NO: 135 - DNA, 100 bp, unidentified
  source 1..100: mol_type = unassigned DNA
tcctgacctccacctcagcctcccaaagttctgggattacaggcatgagccaccgcgcctggccaatttttgtatttttaatagagacggggtttcgcca

SEQ ID NO: 136 - DNA, 100 bp, unidentified
  source 1..100: mol_type = unassigned DNA
aaccaacatctgctgagctgaaaccaccccaggcctgacagcaacagaacgtgtaaaacatgttcacagatcaatttcattcggcttttgcaatgaccgt

SEQ ID NO: 137 - DNA, 100 bp, unidentified
  source 1..100: mol_type = unassigned DNA
attacaggcatgcgccaacaagcccggctagtttttttgtatttttagtagagacgggatttctccatgtggtcaggctggtctcgaactcctgacctca

SEQ ID NO: 138 - DNA, 100 bp, unidentified
  source 1..100: mol_type = unassigned DNA
taaaatctagaattcccagctgggtgcagtaactcatgcctaaaaccccagcagtttgggaggccgaggcgggtagatcacttgaggtcaggagttccac

SEQ ID NO: 139 - DNA, 100 bp, unidentified
  source 1..100: mol_type = unassigned DNA
ctcctgggcgcaaatgatcgacctgcctcggcctccccaagtgttgggattacaggtgggccactgtgcccaagctatttcactatttcatcagcgcact

SEQ ID NO: 140 - DNA, 100 bp, unidentified
  source 1..100: mol_type = unassigned DNA
ttgaatgataattctattcgattccattgaaagattccattcgatggtgattccattagtgtccaatcgatgattccattcgattccgttcgatgatgat

SEQ ID NO: 141 - DNA, 100 bp, unidentified
  source 1..100: mol_type = unassigned DNA
  misc_difference 88: note = a, c, t, g, unknown, or other
  misc_difference 89: note = a, c, t, g, unknown, or other
  misc_difference 90: note = a, c, t, g, unknown, or other
  misc_difference 91: note = a, c, t, g, unknown, or other
  misc_difference 92: note = a, c, t, g, unknown, or other
  misc_difference 93: note = a, c, t, g, unknown, or other
  misc_difference 94: note = a, c, t, g, unknown, or other
  misc_difference 95: note = a, c, t, g, unknown, or other
  misc_difference 96: note = a, c, t, g, unknown, or other
  misc_difference 97: note = a, c, t, g, unknown, or other
  misc_difference 98: note = a, c, t, g, unknown, or other
  misc_difference 99: note = a, c, t, g, unknown, or other
  misc_difference 100: note = a, c, t, g, unknown, or other
ccggtggctcacgcctgtaatcccagcactttgggaggccgaggcgggcggatcacgaggtcaggagatcgagaccatcccggctaannnnnnnnnnnnn

SEQ ID NO: 142 - DNA, 100 bp, unidentified
  source 1..100: mol_type = unassigned DNA
tccagaggccagaagggagagagggtggggaggaccgaaggtgggcgccaggccccagaatgccagtgccctccgtctgacgctccctcttccctggggc

SEQ ID NO: 143 - DNA, 100 bp, unidentified
  source 1..100: mol_type = unassigned DNA
aatgtaatggactctaatagaatggtctcaaatataatggactcgaaagaaattgtctcgaatggaatttattcaaatggaatggaatcgaatggaatgc

SEQ ID NO: 144 - DNA, 100 bp, unidentified
  source 1..100: mol_type = unassigned DNA
aatggaactgaatggcgtcgaatggaattgaatccaatggaatggcattgaatggaatggaaaggaatggaatggaattgaaaggaatagaaaggaatgg

SEQ ID NO: 145 - DNA, 100 bp, unidentified
  source 1..100: mol_type = unassigned DNA
gaaaggaatagaatggaatggaatggacccaaatgtaatggactcgaattgaatggactcaaatagaatggattcgaaaggaatggtctcgaatggaatt

SEQ ID NO: 146 - DNA, 100 bp, unidentified
  source 1..100: mol_type = unassigned DNA
ggcatcgaatggcatcgaatggaatggaatggaaagaaatagaatggaatggacccaaatataatggactcgaatggaatggactccaatagaatggact

SEQ ID NO: 147 - DNA, 100 bp, unidentified
  source 1..100: mol_type = unassigned DNA
gaaacaatagaatggaatggaatcggatggaatgcaatggaatggaatggagtcgagtggaatagaatcgaatggaatggcatcgaatggaatgtaattc

SEQ ID NO: 148 - DNA, 100 bp, unidentified
  source 1..100: mol_type = unassigned DNA
cggaatgtaatcaaaccgagtggaatggaatggaatggaatgaaatggtacggaatggaatcaagacaagtggaatggaatggaatgaaatggtatggaa

SEQ ID NO: 149 - DNA, 100 bp, unidentified
  source 1..100: mol_type = unassigned DNA
tggaatggaatggaatcaacccgagtcgaatggaaaggaatggaatgcaatggactataatgtgatcaactggaatggaatggaatggaatgcaatggaa

SEQ ID NO: 150 - DNA, 100 bp, unidentified
  source 1..100: mol_type = unassigned DNA
atcccagctactcaggaggctgaggcaggagaatcacttgaactcgggaggcggaggttgtggtgagccgagatcgcgacactgcgctccaacctgggca

SEQ ID NO: 151 - DNA, 100 bp, unidentified
  source 1..100: mol_type = unassigned DNA
gactgggcgggcagtacctggggagcgcagcttggtctggctggccgagcgggagagaggcaggctctgtcggaagcggttcatcctgctgaagcccagg

SEQ ID NO: 152 - DNA, 100 bp, unidentified
  source 1..100: mol_type = unassigned DNA
ccacgcaccttccaaggagggagggcaacccacgggtccacaccggctccttaatcccctggctgtgctgcaccggtgcacacacccacctctcaactcc

SEQ ID NO: 153 - DNA, 100 bp, unidentified
  source 1..100: mol_type = unassigned DNA
catgaacacgagtcagcgtaaacactcgtcccccgggctggggctaagccactcgcccctcctcgggcgcctgcccccaccgcgatgtacatgcatgacc

SEQ ID NO: 154 - DNA, 100 bp, unidentified
  source 1..100: mol_type = unassigned DNA
gggggaggggaggaggcgtttctcagggatcgggaacctgcaatggcctggacgtcccccacccctggatggctctcggtgccgcggagcgggcccccat

SEQ ID NO: 155 - DNA, 100 bp, unidentified
  source 1..100: mol_type = unassigned DNA
tggagaggcgcccggggagaggcaggggctgcccagcaccccactagggactgaagggggaagtgcgtggatggcgtggaaagcgcgcctacggccccta

SEQ ID NO: 156 - DNA, 100 bp, unidentified
  source 1..100: mol_type = unassigned DNA
ctcagccttctaaagtggcgggattacacgcatgagtcaccgcgcccagccgggaatgcatgcttttaggagaaacgaagacaactcaggcccctcattc

SEQ ID NO: 157 - DNA, 100 bp, unidentified
  source 1..100: mol_type = unassigned DNA
cgcggcagcagccccagcattgggtcggccggggcggacgggggcggctctccgcccggctgcatttgcatgtcggtttagtcaccgccgccggcgccta

SEQ ID NO: 158 - DNA, 100 bp, unidentified
  source 1..100: mol_type = unassigned DNA
gaacaaccccagtcataaaatcatcatctccgtgatcaccaccagaccatcagcagccaaccaaccccctaaaacagctccggctttacagttcttgggt

SEQ ID NO: 159 - DNA, 100 bp, unidentified
  source 1..100: mol_type = unassigned DNA
cgatctcggctcactgcaacacccgcttcccgggtccaagcgatcctcctgtctcagcctcctgagaagctgggattataggcacaggccaccatgcctg

SEQ ID NO: 160 - DNA, 100 bp, unidentified
  source 1..100: mol_type = unassigned DNA
atctggcagggtgttcgctgcgcttctggtccagtgaggcgcccactgccgctcccgatgctgatcgtcgccattgcagacccactgttgacttccaccc

SEQ ID NO: 161 - DNA, 100 bp, unidentified
  source 1..100: mol_type = unassigned DNA
gcagtagcatttccccatcctggaaatgagggtgtgtggggcgctggactgtagaggcctgagatccaacccggcagagccagcgggggagcgccccgcc

SEQ ID NO: 162 - DNA, 100 bp, unidentified
  source 1..100: mol_type = unassigned DNA
agcaaacattattaggtaatattgaaataacgtaatagttgatctcctggaaaggtcaccagcactacccagaattacttattgaaatgccatttttagg

SEQ ID NO: 163 - DNA, 100 bp, unidentified
  source 1..100: mol_type = unassigned DNA
caataatttaatcatgatagtggtaagtactaaaaaggagaaaaatagcttggcaacccggaaactcttcagaattagccccctttttaaccattgttgt

SEQ ID NO: 164 - DNA, 100 bp, unidentified
  source 1..100: mol_type = unassigned DNA
aggctgtgtctctgtttagaactgaacacgagggtggggagtgggggatgctggaccacagggcagcacctgcccggcaggatgcgcgtgcctgtgagcc

SEQ ID NO: 165 - DNA, 100 bp, unidentified
  source 1..100: mol_type = unassigned DNA
ccatgcctggctaattttaaaatcttttttttggtagagacgagcgtctcactgtgttgtccaggctggtctcaaactcctgtcctccagtgatcctccc

SEQ ID NO: 166 - DNA, 100 bp, unidentified
  source 1..100: mol_type = unassigned DNA
cctggcagccactggtctttctgctgtctccgtggtttcgtcttttcaagagtgtcatacccagtaaggcttcatttcatatcattgataggtttttgga

SEQ ID NO: 167 - DNA, 100 bp, unidentified
  source 1..100: mol_type = unassigned DNA
aatgctgagttgtcttacggccctccctggcaaacctgtgactggccccgggacagtgtccttggccaggagggcagccaccccccaccctgcctctctc

SEQ ID NO: 168 - DNA, 100 bp, unidentified
  source 1..100: mol_type = unassigned DNA
gaggtaaccgctccctggacagcgggtggctcacagcacgtgctaggacccgtccactgctccttagcaggcatgacgctaaccctccccacctgcttgc

SEQ ID NO: 169 - DNA, 100 bp, unidentified
  source 1..100: mol_type = unassigned DNA
ccctcgcacctaggcagaaggtccattgccctctgccccgcagcctgcccgttcccaggccagcgagggcgctcccaggccggtctgcagccagaacaca

SEQ ID NO: 170 - DNA, 100 bp, unidentified
  source 1..100: mol_type = unassigned DNA
gataggagttgaaggggcacgctaaaaagtgaccaaaaaacgagtgtgagctctttgtcaggcctgggcagggccactagagggctccttggtctagcgg

SEQ ID NO: 171 - DNA, 100 bp, unidentified
  source 1..100: mol_type = unassigned DNA
taaggccagcgtctgggaaggcgcccgttaccaagcggagcatggtctatggtagtgtcagtgccaaagaaaagcatccgctacttagcagatcggaaaa

SEQ ID NO: 172 - DNA, 100 bp, unidentified
  source 1..100: mol_type = unassigned DNA
aggcaaggattgcttgaacccaggagttcaagaccaggctgggaaaacccagcaagacccgtctcgacaaaaaatttaaaaactagccgggtgtagtggc

SEQ ID NO: 173 - DNA, 100 bp, unidentified
  source 1..100: mol_type = unassigned DNA
cggcggcggcggtggcggcggcggcggcggcggcgggaccgaaaccccccccgagtgttacagcccccccggcagcagcactcgccgaatcccggggccg

SEQ ID NO: 174 - DNA, 100 bp, unidentified
  source 1..100: mol_type = unassigned DNA
tatcacaccacctagtcgttcaaatcctgcttatttgcccaagacaaatgaaagcgtatgtacaaacgattggacaaacattcgtagcaactttatttga

SEQ ID NO: 175 - DNA, 100 bp, unidentified
  source 1..100: mol_type = unassigned DNA
ctccctcagcctcccgagtagctgggattacaggtacgcatcaccacacccagctaatttttggatttttagtagagactaggttttaccatgttggcta

SEQ ID NO: 176 - DNA, 100 bp, unidentified
  source 1..100: mol_type = unassigned DNA
gattatttcattcgattccattcgatgattccaattgattccattcgataatgattccattcgagtccattcaattattccttttgagtccaatccatga

SEQ ID NO: 177 - DNA, 100 bp, unidentified
  source 1..100: mol_type = unassigned DNA
ttccattcgagtccgttcgatcattccatttgagtccattcgatgaagattccattcgagtccattcaataattctttttaagtccatttgatgatgatt

SEQ ID NO: 178 - DNA, 100 bp, unidentified
  source 1..100: mol_type = unassigned DNA
cgtatcctttcaatgattccatttgattccattcaatgatgattccactcgagtccatttgatgattccattcgattccatttgatgattcctttcgatc

SEQ ID NO: 179 - DNA, 100 bp, unidentified
  source 1..100: mol_type = unassigned DNA
tggcttccattcgatgatgattcctttcgaatactttcaatgattccattcgagtccattcgatgatgattccatttgagttcattctatgattccattc

SEQ ID NO: 180 - DNA, 100 bp, unidentified
  source 1..100: mol_type = unassigned DNA
attccattcgatgattctgtttgattccattcaatgatgattcccttctattccattcgatgattccgttcgattccattctgtgatgattctattcgat

SEQ ID NO: 181 - DNA, 100 bp, unidentified
  source 1..100: mol_type = unassigned DNA
ttctattcgattccattcaatgatgattccatcagattccattcgatggttccatttgattccattcagtgatggttccattcgattccattcgatgatg

SEQ ID NO: 182 - DNA, 100 bp, unidentified
  source 1..100: mol_type = unassigned DNA
cctcccgagtagctgggactacaggcgcccgccaccacgcctggctaattttttgtatttttagtagagacggggtttcaccctgttagccaggatggtc

SEQ ID NO: 183 - DNA, 100 bp, unidentified
  source 1..100: mol_type = unassigned DNA
ggcctggacaggcttagtgacccttgcaccagaactctccggacaccgccccttcgcccactcgttaggctcctctgccttcgggccggctgggctgccc

SEQ ID NO: 184 - DNA, 100 bp, unidentified
  source 1..100: mol_type = unassigned DNA
caagcaattctcctgtctcagcctcccgagtaattgggactacaggcatgcacctcgatatccggctaatttttgtatttttagtagagacggggtttca

SEQ ID NO: 185 - DNA, 100 bp, unidentified
  source 1..100: mol_type = unassigned DNA
gccgcttgccttgggaacgcggcccgaagcccaggacctggtagatgggcgcaggcgggcggtcggccgtgtcctcgccgcgggtcaccatcgcctcgcg

SEQ ID NO: 186 - DNA, 100 bp, unidentified
  source 1..100: mol_type = unassigned DNA
agcaaaacctctaatcattaattttggtcaagtttaaaacagagattcccatttatggcgcacgtcggtataaacacggcagctttataaaaattcccat

SEQ ID NO: 187 - DNA, 100 bp, unidentified
  source 1..100: mol_type = unassigned DNA
gatatatacgccctatgcagagcccggctccaacgcctcttgtagaaagaaggacactgaccaggacatttgcagcgtcacaaaagatcctgtcagcgcg

SEQ ID NO: 188 - DNA, 100 bp, unidentified
  source 1..100: mol_type = unassigned DNA
ccaggctgactggctggaaaagaaaaaaaaaaaacgcggagttcgacgccgcgcgcagcaccaaacacttccccagaagggcgcagcctggaggcggccg

SEQ ID NO: 189 - DNA, 100 bp, unidentified
  source 1..100: mol_type = unassigned DNA
cgcgcaggacgcacgcccaaggcgcctgggccgctagggaccgaccggagcgctcaaacccacagggtatctatcaggcgcgcgtcagcacgtaagcaga

SEQ ID NO: 190 - DNA, 100 bp, unidentified
  source 1..100: mol_type = unassigned DNA
gcttcccattggttgagacagcaccgcccagccaaagcccccttgtcctcgcgcgggtgcgccgcctggactcccaccctggccagtcccgggcccacca

SEQ ID NO: 191 - DNA, 100 bp, unidentified
  source 1..100: mol_type = unassigned DNA
ccactctggcatccccagcctgtccccgcggctcctcctccttccccggccctgtggcctgcctgctgtccagcattcttccccagggcgctgtggcgcg

SEQ ID NO: 192 - DNA, 100 bp, unidentified
  source 1..100: mol_type = unassigned DNA
ctcctgcctggcacattattccgaataataaatgccagatcatgctgctggagacgccggcagaaaagccgagagggagactccctggggtctgggcgcg

SEQ ID NO: 193 - DNA, 100 bp, unidentified
  source 1..100: mol_type = unassigned DNA
atcggccatcctacccttaccccgctgcggccgcagggctcggcctgtagggcgcgagccccccttccccacccgtatcccggcttcggagctaaaggaa

SEQ ID NO: 194 - DNA, 100 bp, unidentified
  source 1..100: mol_type = unassigned DNA
cacgtgaggccgaaccaaaccgagtagagggcgccagggaatcccgcccgagctgcagagacgcggacccctcctcttacccaaaaccaaatgggcaggg

SEQ ID NO: 195 - DNA, 100 bp, unidentified
  source 1..100: mol_type = unassigned DNA
ggctccgctgcagagggctcgccgaggctctcgccttcacccggcgccgcgcaggtgcccgctctcctgcggggagagccgagggggggcgctgccgcag

SEQ ID NO: 196 - DNA, 100 bp, unidentified
  source 1..100: mol_type = unassigned DNA
gggctcacggcggcgtggctgtcggggggccgccgagccgcctcctccgcgctgggtgcccggctggcgctcctcccgcccaaggtgccctgatcgctag

SEQ ID NO: 197 - DNA, 100 bp, unidentified
  source 1..100: mol_type = unassigned DNA
gctcccggtgcggccgtctcgcccctaagccgggcgccagatcccccgccgccttctcgctccccgaccgctgggcgggggcgagggccagggagatgga

SEQ ID NO: 198 - DNA, 100 bp, unidentified
  source 1..100: mol_type = unassigned DNA
gccaggtgcggaaagggttgcagcctcgcgggctgaggccggagggtgaaggcgccgctccttgaagtgaggaaaactccggtgaggaagaggctctttc

SEQ ID NO: 199 - DNA, 100 bp, unidentified
  source 1..100: mol_type = unassigned DNA
tgattcttagaaaagatcaataaattgactgacctctagcaaaactgataaaaacaaaaagaaacacgaacaagttaccaatatcagggagaagaaaaag

SEQ ID NO: 200 - DNA, 100 bp, unidentified
  source 1..100: mol_type = unassigned DNA
gaaaaactggaaactctaaaacgcagagcgcctctcctcctccaaaggaacgcagttcctcaacagcaacagaacaaagctggatggagaatgattttga

SEQ ID NO: 201 - DNA, 100 bp, unidentified
  source 1..100: mol_type = unassigned DNA
ttctcttccctaaattgatattttcagaatgatagaggttttccttttaattggtagatttaaaaaataaatcggcctagattcatagaatagaaatgac

SEQ ID NO: 202 - DNA, 100 bp, unidentified
  source 1..100: mol_type = unassigned DNA
cgatcactgcagtagcatagatagatcaattctatctcaggtcatttgcctggaactcttcccccagatcttcccttggcagagcttcatatcccagctt

SEQ ID NO: 203 - DNA, 100 bp, unidentified
  source 1..100: mol_type = unassigned DNA
agttcaaggccagcctgcccaacatggtgaaacctcatctccactaaaattacaaaagttagccaggcgtggtggtgcacacttgtggtcccagctactc

SEQ ID NO: 204 - DNA, 100 bp, unidentified
  source 1..100: mol_type = unassigned DNA
tcagtagttttttacaccattaaaacattttatcttaccttttgtgggatcttggcaagggctgatgcaattacattggccctttcttctgacgtgttac

SEQ ID NO: 205 - DNA, 100 bp, unidentified
  source 1..100: mol_type = unassigned DNA
aacctaatgttgtgtttgaaattgtataggccaatctaaattattgcaattcggtattaaaccagtaacaaattccacatgtgaaaaggacaagaataga

SEQ ID NO: 206 - DNA, 100 bp, unidentified
  source 1..100: mol_type = unassigned DNA
cttacgtgaagatatttctttttcgaccatggacaagaaagcactccaaatgaacacttggagattctacaaaaagtgtgtttcaacactgctctatcaa

SEQ ID NO: 207 - DNA, 100 bp, unidentified
  source 1..100: mol_type = unassigned DNA
caaatctgtaagtcgatattcggacctattagaggccttcgttggaaacgggatttcttcatataatgctagaaagaagaattctcagtaacttccttgt

SEQ ID NO: 208 - DNA, 100 bp, unidentified
  source 1..100: mol_type = unassigned DNA
caactcacagagttgaacgatcgtttccccagagcagacttgaaacactctttttgtggaatttgcaaatggagatttcagccgctttgaggtcaatggt

SEQ ID NO: 209 - DNA, 100 bp, unidentified
  source 1..100: mol_type = unassigned DNA
cgattcccctgcctcagcctcctgagtagctgggactataggcgaccgccaccacgcctggctaatttttttttttttttttttttgtattttagtacag

SEQ ID NO: 210 - DNA, 100 bp, unidentified
  source 1..100: mol_type = unassigned DNA
gcgggatctcggcttactgcaaactccgcctcccgggttcacgccattctcctgcctcagcctcccgagtagatgggactaaggcgcccgccaccgtgcc

SEQ ID NO: 211 - DNA, 100 bp, unidentified
  source 1..100: mol_type = unassigned DNA
ttttgtatttgcgctcggtttgtctttgcaatgaccttgaagctagggagactgacttgagaagcctcggcgacttgccttcacttccatcagtaaagcc

SEQ ID NO: 212 - DNA, 100 bp, unidentified
  source 1..100: mol_type = unassigned DNA
ggatattaaaaggtttacgtttatataaaatggacaaaaatacacatttgcagtttgacctcagggctgttaagttagcaaactctttgttgacatagtt

SEQ ID NO: 213 - DNA, 100 bp, unidentified
  source 1..100: mol_type = unassigned DNA
aatctactgtcctgtctacttctttctccttgatgcccccagggccccatctcctacggtaggttgtttttaattattgattagacaaatccaactttaa

SEQ ID NO: 214 - DNA, 100 bp, unidentified
  source 1..100: mol_type = unassigned DNA
gagctttaggaaggtcaaatgccacagctcctccgcctcccgcacctacaatccaggagaactgaccttgagcactggtcattccagaggattgtgtaaa

SEQ ID NO: 215 - DNA, 100 bp, unidentified
  source 1..100: mol_type = unassigned DNA
ggcatttgtgtgcgtaaccccgggtagcgttcatgtgcacctaaagtgtgacgacagcctatgggaaaccaagggaagggtcacctgtagaggtccggac

SEQ ID NO: 216 - DNA, 100 bp, unidentified
  source 1..100: mol_type = unassigned DNA
ggctctgccacctgccaagtgtctgatctgaggacaagtggtgctttctgggcctcagtgtcttcatctgcaaaacgggaccgtgagaccacacctgact

SEQ ID NO: 217 - DNA, 100 bp, unidentified
  source 1..100: mol_type = unassigned DNA
taggttctgaagccttggaccaggggaccggccggagcttctgtgcctgagaatgctgagccggtccagcatggcccaggtcgattgtgcaatcctgggc

SEQ ID NO: 218 - DNA, 100 bp, unidentified
  source 1..100: mol_type = unassigned DNA
cctctgccacctggccttggtagacgcgggcttcactactagcgtggtgccgccgctgctggccaacctgcgcggaccagcgctctggctgccgcgcagc

SEQ ID NO: 219 - DNA, 100 bp, unidentified
  source 1..100: mol_type = unassigned DNA
cctggaccgcggcggacatggcggctcagatcaccaagcgcaagtgggaggcagaagaatttgcagagcagatcagggcctacctggagggcacgtgcgt

SEQ ID NO: 220 - DNA, 100 bp, unidentified
  source 1..100: mol_type = unassigned DNA
tgaaaagcagggctgaggagtggcggcaaccggcagcgtccagctcccgcacctcgctgcacatcgcacctgagccccgccgcgaccgcatcgcgctcgc

SEQ ID NO: 221 - DNA, 100 bp, unidentified
  source 1..100: mol_type = unassigned DNA
gctgttgatgtctgcgcgctcctccctctaggggtcatcactctggaccctcagaccgccagccggagcctggttctctcggaagacaggaagtcagtga

SEQ ID NO: 222 - DNA, 100 bp, unidentified
  source 1..100: mol_type = unassigned DNA
gacggcgtctgggccgtgatcatctcgcaccagcagtgctgggccagcacctccccgggcaccgacctgccgctgagcgagatcccgcgcggcgtgagag

SEQ ID NO: 223 - DNA, 100 bp, unidentified
  source 1..100: mol_type = unassigned DNA
tttctttgccgtctggaaaaaaggttcctgccttacgctgaaaggctgaagtggggcgcgcgaagggcggcgaagcggagacggcggctctccgggatcc

SEQ ID NO: 224 - DNA, 100 bp, unidentified
  source 1..100: mol_type = unassigned DNA
ccaaatgccttaaggacggcctacatactaaggaaaatttttttctaactcctggttgcagctgaggggagcggctgagggcggggacaggggtgcggcg

SEQ ID NO: 225 - DNA, 100 bp, unidentified
  source 1..100: mol_type = unassigned DNA
cctcggagtacgccgcaccatgcagctcaggctcttccggcgcctccttctcgccgctttgctgctggtgatcgtctggaccctcttcgggccttcgggg

SEQ ID NO: 226 - DNA, 100 bp, unidentified
  source 1..100: mol_type = unassigned DNA
tctgcctcccaggttcaattgattctcctgcctcagctgggattacagtcacctgccaccacacccagctaagttttgtatttttagtagagacggggtt

SEQ ID NO: 227 - DNA, 100 bp, unidentified
  source 1..100: mol_type = unassigned DNA
gcagtggatctcccagcacagcgttcaagctctgataaggaacagactgcgtcctcaagtgagtccctgacccctgtgtatcctgactgggagacacttc

SEQ ID NO: 228 - DNA, 100 bp, unidentified
  source 1..100: mol_type = unassigned DNA
attcgttataaaatccattagttctactgatattcataatttcctttctgaagaaaaaactatctgaatagttggtttttttaggccaacagtacactga

SEQ ID NO: 229 - DNA, 100 bp, unidentified
  source 1..100: mol_type = unassigned DNA
ggctccatagtgactattaggatgaagatggactggaggtagggggtagaaaggagatgtgttctctattcgcaggaaatcaataatttacgcaaacgtt

SEQ ID NO: 230 - DNA, 100 bp, unidentified
  source 1..100: mol_type = unassigned DNA
aattctttgtgtagtgtgcattcaactcacagaatggaacgtccctttagacagagcagatttgaaacactctttttgcgcaatttgcaagtggagattt

SEQ ID NO: 231 - DNA, 100 bp, unidentified
  source 1..100: mol_type = unassigned DNA
cactctgtcgcccaggctggagtgcagtggcacgatctcggctcactgcaacgtccccctcccgggttcaagcgattctcctgcctcagcctcctgagta

SEQ ID NO: 232 - DNA, 100 bp, unidentified
  source 1..100: mol_type = unassigned DNA
cttgcccggtcgcccaggctggagcgcagtggcacaatctcggctcactgcaagctccgcctcccgggttcacgccattctcctgcctcagcctccccag

SEQ ID NO: 233 - DNA, 100 bp, unidentified
  source 1..100: mol_type = unassigned DNA
agcctcccacgtcagcctcccgagtaggtgggatcacaggcgcacatcacctcacccggcaaattttaaaaattatttggccaggcgcagtggctcacgc

SEQ ID NO: 234 - DNA, 100 bp, unidentified
  source 1..100: mol_type = unassigned DNA
atcgcccaggctggagtgcagtggcgtgatctcggctcactgcaaccttcgcctcccaggttccagagattctcctgtctcaggctcccaagtagctggg

SEQ ID NO: 235 - DNA, 100 bp, unidentified
  source 1..100: mol_type = unassigned DNA
agctgggcatggtggtgccttctcctgaggcaggagaatcgcttgaacccaggaggtggaggctgcagtaagctgagatcgcgccactgcactccagcct

SEQ ID NO: 236 - DNA, 100 bp, unidentified
  source 1..100: mol_type = unassigned DNA
agctacaggcgcgcgtcatcatgcccagctaatttttgtatttttggtagagacggggtttcaccatgttgaccaggcttgtctcgaactcctgacctca

SEQ ID NO: 237 - DNA, 100 bp, unidentified
  source 1..100: mol_type = unassigned DNA
tgaaatattttgtaatgtttttacaataataaaatttatacactaagatgcttaaacgtgagtagtaaaatctaggtatcattttcaaactaaaatatta

SEQ ID NO: 238 - DNA, 100 bp, unidentified
  source 1..100: mol_type = unassigned DNA
cctgtttgcctgggtgtcagcagcagtggctgtagaacagcggattttcgtgagccacgaatgctgctgtctgatcgttcctctggaagttttgtcgcag

SEQ ID NO: 239 - DNA, 100 bp, unidentified
  source 1..100: mol_type = unassigned DNA
gttttgtgtgtgtgtttgttttttgtttgtttgttttgagatggagtctcactctgtcacccaggctggagtgcagtggcgcaatcttggctcactgcaa

SEQ ID NO: 240 - DNA, 100 bp, unidentified
  source 1..100: mol_type = unassigned DNA
ccagctacttgggaggctgaggcaagagaatagcttgaacctgggaggcagaggttgcagtgagccgagattgcgccactgcactccagcctgggtgaca

SEQ ID NO: 241 - DNA, 100 bp, unidentified
  source 1..100: mol_type = unassigned DNA
cccggcactgaccagccctgccctcccatgcccctgcccaggccacaagcgttccacgagcgaaggcgcctggccacagctgccctctggcctctccatg

SEQ ID NO: 242 - DNA, 100 bp, unidentified
  source 1..100: mol_type = unassigned DNA
ctgcccggcgcagggggtcctgtggactccgcaggtagccctgggtctggctcaggaagttgggcacgtggcctgggtatcgctgaacctggggacaaaa

SEQ ID NO: 243 - DNA, 100 bp, unidentified
  source 1..100: mol_type = unassigned DNA
tctcatcccagcatcccacccactgtcagcacccgccccggccccccgcactgtccccataccccaccccgctgtcagctgcctctcccgcccgcctccc

SEQ ID NO: 244 - DNA, 100 bp, unidentified
  source 1..100: mol_type = unassigned DNA
ggactttgccccgcaatgatttcctgtcgttgaaaaaactttcaatctaactttcccccgaaatgatacaagcagggtttattttggtatttttaatgta

SEQ ID NO: 245 - DNA, 100 bp, unidentified
  source 1..100: mol_type = unassigned DNA
agtccctccagaggttcggaagactcacgaccccaaaacatgatgtttcccatatgtgggctcatcctgagacgcagccatcactatccaattgtccctg

SEQ ID NO: 246 - DNA, 100 bp, unidentified
  source 1..100: mol_type = unassigned DNA
taagaaaggagaaatcacgtctgaggctgatttctaagccctcagatctttaaacagtcatgtgtattttaaatatattaagaggaaaaattatattttt

SEQ ID NO: 247 - DNA, 100 bp, unidentified
  source 1..100: mol_type = unassigned DNA
ccatcgcaaagaagctaaaaaccttgaaaaaggattagacgaatggctaactagaataaacagcgtagagaagaccttaaatgacctgatggagctgaaa

SEQ ID NO: 248 - DNA, 100 bp, unidentified
  source 1..100: mol_type = unassigned DNA
tgagacagagtcttgctctgtcgcgcaggctggagtgcagtggcgcgatctcggctcactgcaacctccacctcctgggttcaagaaattctcttgcctc

SEQ ID NO: 249 - DNA, 100 bp, unidentified
  source 1..100: mol_type = unassigned DNA
tttcgcttggctctcgtgctcccttgcaggccgccatggtaagacgtacctttcaccttcagccgtgattgtgaggcctccccagccacgtggaactgtg

SEQ ID NO: 250 - DNA, 100 bp, unidentified
  source 1..100: mol_type = unassigned DNA
ggaggaggaggttgcagtgagcccagatggtgccagtacactctaggctgggcgacagagaaagactccgtcttaaaaaaaaaaaaaagaaagaaagaaa

SEQ ID NO: 251 - DNA, 100 bp, unidentified
  source 1..100: mol_type = unassigned DNA
tttttgagatggaattttgctcttgttgtccagactggagtgcagtggcacgatctcggctcaccacaacctccgcctctcgggttcaagcgattctcct

SEQ ID NO: 252 - DNA, 100 bp, unidentified
  source 1..100: mol_type = unassigned DNA
aaaaaaaattaaccgggttttgtggcgggtgcctgtagtcccagctactcgggaggctgaggcaggagaatggcatgaacccgggaggtggagcttgcag

SEQ ID NO: 253 - DNA, 100 bp, unidentified
  source 1..100: mol_type = unassigned DNA
gttctgtcacccaggctggagtgcaatggcgcgatctccgctcacagcaacctccaccacccggattgaagcgattctcttgcctcagcctcccgagtag

SEQ ID NO: 254 - DNA, 100 bp, unidentified
  source 1..100: mol_type = unassigned DNA
acccaccttggcctcctaaagtgtagggattacaggcgtgagccaccatgcccggccccaagtcaagtctttttcacagccttggttcaccataaaacta

SEQ ID NO: 255 - DNA, 100 bp, unidentified
  source 1..100: mol_type = unassigned DNA
ggtcgtggtggcaggcacctgtaatcccacctgctctggaggccgagacaggagaatcacttgaacccgggacgtgggcgttgcagtgagctgagatcgc

(19) United States
(12) Patent Application Publication
(10) Pub. No.: US 2025/0027164 A1
(43) Pub. Date: Jan. 23, 2025

(54) METHODS AND COMPOSITIONS FOR TREATING LIVER CANCER AND LIVER DISEASE

(71) Applicant: Active Genomes Expressed Diagnostics Corp, Bethesda, MD (US)

(72) Inventor: Rachel ZAYAS, Washington, DC (US)

(21) Appl. No.: 18/423,938

(22) Filed: Jan. 26, 2024

Related U.S. Application Data
(63) Continuation of application No. PCT/US2022/074339, filed on Jul. 29, 2022.
(60) Provisional application No. 63/227,548, filed on Jul. 30, 2021.

(51) Int. Cl. C12Q 1/6886

(52) U.S. Cl. CPC C12Q 1/6886; C12Q 2600/154

(57) ABSTRACT

Methods, compositions, and kits for determining a concentration of at least one methylation signature associated with a disease (e.g., HCC, NAFLD, NASH, fibrosis) in a subject suspected of having NAFLD or HCC are provided. In some instances, the methods comprise obtaining a tissue sample comprising a nucleic acid from the subject, isolating the nucleic acid from the tissue sample, determining a concentration of a disease-specific methylation signature in the tissue sample by contacting the nucleic acid with at least one disease-specific (NASH specific, HCC specific) methylation signature or liver specific methylation signature detection molecule, and determining if the disease specific methylation signature or liver specific methylation signature is detected in the tissue sample.

### CROSS-REFERENCE TO RELATED APPLICATIONS

This application is a continuation of International Application No. PCT/US2022/074339, filed Jul. 29, 2022, which claims the benefit of U.S. Provisional Application No. 63/227,548, filed Jul. 30, 2021, each of which is incorporated by reference herein in its entirety.

### SEQUENCE LISTING

The instant application contains a Sequence Listing which has been submitted electronically in XML format and is hereby incorporated by reference in its entirety. Said XML copy, created on Apr. 29, 2024, is named 65693-701_301_SL.xml and is 261,059 bytes in size.

### BACKGROUND

Hepatocellular carcinoma (HCC) causes 800,000 deaths globally and is the sixth leading cause of cancer related mortality in the US.¹⁻³ The incidence of HCC in the US has tripled in the last two decades, with projections to continue rising. The gold standard FDA-approved blood test for early detection of HCC, Alpha-fetoprotein (AFP), is less than 60% accurate, is often elevated in other cancers and cannot be used alone to diagnose HCC. At the time of diagnosis, the majority of patients will not live beyond a year.²⁻⁵ While improvements in identification of cell surface markers to isolate hepatocytes and derived material from hepatocytes in liquid biopsy have been made, but they are insufficient to be used in clinical settings.

Hepatocyte isolation techniques have relied on certain mutations and used techniques such as antibody affinity bead capture or droplet-based microfluidics to separate liver cells from other cell matter in liquid biopsy.⁶ The majority of markers rely on epithelial cell adhesion molecules (EpCAM) to target circulating tumor cells (CTC).^{6,7} However, EpCAM is not a universal marker for liver cancer. First, hepatocytes undergo epithelial to mesenchymal transition and EpCAM fails to account for cancer cells of mesenchymal origin.^{6,7}

Additionally, EpCAM are found in other tissues in the body (such as breast or kidney tissue) and have also been found in benign tumors. Another complicating factor is that CTC are challenging to target in early stages of HCC due to low enumeration of CTCs.^{8,9} Although the role of HCC is well studied, the current available information is not yet predictive for risk assessment of HCC. The current landscape is devoid of a sensitive technique for hepatocyte specific markers and these challenges must be addressed to assess HCC progression early and through non-invasive methods.

What is needed are methods and compositions to identify the origin of cellular components and determine the tumorigenicity and other disease states of the tissue of origin.

### SUMMARY

Aspects described herein provide a first method of determining if an HCC specific methylation signature is detected in a subject suspected of having liver cancer, by (a) obtaining a tissue sample comprising a nucleic acid from the subject; (b) isolating the nucleic acid from the tissue sample; (c) determining a concentration of the HCC specific methylation signature in the tissue sample by contacting the nucleic acid with at least one HCC specific methylation signature detection molecule; and (d) determining if the HCC specific methylation signature is detected in the tissue sample.

Further aspects provide a second method of determining a difference in a beta value between an HCC specific methylation signature in a tissue sample and a control tissue sample by determining a beta value of the HCC specific methylation signature in a nucleic acid from the tissue sample, determining a beta value of the HCC specific methylation signature in a nucleic acid from the control tissue sample, and determining if a difference in an average beta value between the beta value of the HCC specific methylation signature in the tissue sample and the beta value of the HCC specific methylation signature in the control tissue sample is greater than or equal to 10%.

Aspects described herein provide a third method of determining if a liver specific methylation signature is detected in a subject suspected of having liver cancer, by (a) obtaining a tissue sample comprising a nucleic acid from the subject; (b) isolating the nucleic acid from the tissue sample; (c) determining a concentration of the liver specific methylation signature in the tissue sample by contacting the nucleic acid with at least one liver specific methylation signature detection molecule; and (d) determining if the liver specific methylation signature is detected in the tissue sample.

Aspects described herein provide a fourth method of determining a difference in a conserved beta value for a liver specific methylation signature by determining a conserved beta value of the liver specific methylation signature in a nucleic acid from a tissue sample, determining a conserved beta value of the liver specific methylation signature in a nucleic acid from a control tissue sample, and determining if a difference in the conserved beta value between the conserved beta value of the liver specific methylation signature in the tissue sample and the conserved beta value of the liver specific methylation signature in the control tissue sample is less than or equal to 5%.

Aspects described herein provide a fifth method of treating liver cancer in a subject suspected of having liver cancer, by (a) obtaining a tissue sample comprising a nucleic acid from the subject, (b) isolating the nucleic acid from the tissue sample, (c) determining a concentration of an HCC specific methylation signature in the tissue sample by contacting the nucleic acid with at least one HCC specific methylation signature detection molecule, and (d) treating the subject for liver cancer if the HCC specific methylation signature is detected in the tissue sample.

Aspects described herein provide a sixth method of treating liver cancer in a subject suspected of having liver cancer by determining a concentration of an HCC specific methylation signature in a nucleic acid from a tissue sample, determining a concentration of an HCC specific methylation signature in a nucleic acid from a control tissue sample, determining a beta value of an HCC specific methylation signature in a nucleic acid from a tissue sample, determining a beta value of the HCC specific methylation signature in a nucleic acid from a control tissue sample; and treating the subject for liver cancer if the concentration of the HCC specific methylation signature in the nucleic acid from the tissue sample exceeds the concentration of the HCC specific methylation signature in the nucleic acid from the control tissue sample, and the beta value of the HCC specific methylation signature in a nucleic acid from the tissue sample is 10% or greater than the beta value of the HCC specific methylation signature in a nucleic acid from the control tissue sample.

Further aspects described herein provide a seventh method of treating liver cancer in a subject suspected of having liver cancer, by (a) obtaining a tissue sample comprising a nucleic acid from the subject, (b) isolating the nucleic acid from the tissue sample, (c) determining a concentration of a liver specific methylation signature in the tissue sample by contacting the nucleic acid with at least one liver specific methylation signature detection molecule, (d) determining a concentration of a HCC specific methylation signature in the tissue sample by contacting the nucleic acid with at least one HCC specific methylation signature detection molecule; and (e) treating the subject for liver cancer if the liver specific methylation signature is detected in the tissue sample and the HCC specific methylation signature is detected in the tissue sample.

Aspects described herein provide an eighth method of treating liver cancer in a subject suspected of having liver cancer by determining a concentration of an HCC specific methylation signature in a nucleic acid from a tissue sample; determining a concentration of an HCC specific methylation signature in a nucleic acid from a control tissue sample; determining a concentration of an liver specific methylation signature in a nucleic acid from a tissue sample; determining a concentration of a liver specific methylation signature in a nucleic acid from a control tissue sample; determining a beta value of an HCC specific methylation signature in a nucleic acid from a tissue sample; determining a beta value of the HCC specific methylation signature in a nucleic acid from a control tissue sample; determining a beta value of a liver specific methylation signature in a nucleic acid from a tissue sample; determining a beta value of the liver specific methylation signature in a nucleic acid from a control tissue sample; and treating the subject for liver cancer if the concentration of the HCC specific methylation signature in the nucleic acid from the tissue sample exceeds the concentration of the HCC specific methylation signature in the nucleic acid from the control tissue sample, the concentration of liver specific methylation signature in the nucleic acid from the tissue sample exceeds the concentration of the liver specific methylation signature in the control tissue sample, the beta value of the HCC specific methylation signature in a nucleic acid from the tissue sample is 10% or greater than the beta value of the HCC specific methylation signature in a nucleic acid from the control tissue sample, and the beta value of the liver specific methylation signature in a nucleic acid from the tissue sample is 10% or greater than the beta value of the liver specific methylation signature in a nucleic acid from the control tissue sample.

Further aspects described herein provide a first kit comprising at least one liver specific methylation signature detection molecule capable of binding to one or more methylation patterns associated with AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TAC1, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

Aspects described herein provide a second kit comprising at least one HCC specific methylation signature detection molecule capable of binding to one or more methylation patterns associated with POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

Aspects described herein provide a ninth method of detecting at least one HCC specific methylation signature in a tissue sample of a subject by contacting a HCC specific methylation signature detection molecule with nucleic acid obtained from the tissue sample, wherein the HCC specific methylation signature detection molecule is capable of binding to one or more methylation patterns associated with POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

Further aspects provide a tenth method of detecting at least one liver specific methylation signature in a tissue sample of a subject by contacting a liver specific methylation signature detection molecule with nucleic acid obtained from the tissue sample, wherein the liver specific methylation signature detection molecule is capable of binding to one or more methylation patterns associated with AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TAC1, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

Aspects described herein provide an eleventh method of determining if a NASH specific methylation signature is detected in a subject suspected of having NASH by (a) obtaining a tissue sample comprising a nucleic acid from the subject, (b) isolating the nucleic acid from the tissue sample, (c) determining a concentration of the NASH specific methylation signature in the tissue sample by contacting the nucleic acid with a NASH specific methylation signature detection molecule; and (d) determining if the NASH specific methylation signature is detected in the tissue sample.

Further aspects provide a twelfth method of determining a difference in a beta value between a NASH specific methylation signature in a tissue sample and a control tissue sample by determining a beta value of the NASH specific methylation signature in a nucleic acid from the tissue sample, determining a beta value of the NASH specific methylation signature in a nucleic acid from the control tissue sample; and determining if a difference in an average beta value between the beta value of the NASH specific methylation signature in the tissue sample and the beta value of the NASH specific methylation signature in the control tissue sample is greater than or equal to 10%.

Aspects described herein provide a thirteenth method of determining if a liver specific methylation signature is detected in a subject suspected of having fibrosis, by (a) obtaining a tissue sample comprising a nucleic acid from the subject, (b) isolating the nucleic acid from the tissue sample, (c) determining a concentration of a liver specific methylation signature in the tissue sample by contacting the nucleic acid with a liver specific methylation signature detection molecule, and (d) determining if the liver specific methylation signature is detected in the tissue sample.

Aspects described herein provide a fourteenth method of determining a difference in a conserved beta value for a liver specific methylation signature by determining a beta value of the liver specific methylation signature in a nucleic acid from a tissue sample of a subject suspected of having fibrosis, determining a beta value of the liver specific methylation signature in a nucleic acid from a control tissue sample, and determining if a difference in the conserved beta value between the beta value of the liver specific methylation signature in the tissue sample and the beta value of the liver specific methylation signature in the control tissue sample is less than or equal to 5%.

### BRIEF DESCRIPTION OF THE DRAWINGS

FIG. 1 provides an exemplary overview of the methods described herein.

FIGS. 2A-2B provide exemplary overview of the bioinformatics pipelines used in exemplary methods described herein. FIG. 2A: MinFi is a Bioconductor open-source tool than can be used for analyzing Illumina Methylation arrays and the mini Bumphunter tool was used to identify differentially methylated regions within the dataset.^{28,29} FIG. 2B: Bismark is an open-source tool than can be used for analyzing Methylation arrays and WGBS datasets used for statistical modeling.

FIG. 3 provides an exemplary epigenotype heatmap generated through Random Forest Machine Learning Output.

FIGS. 4A-4C provide an exemplary boxplot of unique liver specific methylation patterns and represents a visual representation of average methylation status between liver tissue, blood and various other tissues. FIG. 4A illustrates that methylation status of oligonucleotide sequence associated with miR443682 has a mean methylation status of 33% in the liver (see left figure), while mean methylation status of same oligonucleotide sequence in blood cells averages 95% and finally mean methylation status of other tissues averages 82%. The methylation status found on the liver has no overlap with any other tissue assessed and is thus defined as a liver specific methylation marker. FIG. 4B illustrates that methylation status of oligonucleotide sequence associated with BDH1 has a mean methylation status around 70% in the liver, while mean methylation status in blood and other tissues is less than 5%. FIG. 4C illustrates the methylation status of oligonucleotide sequence associated with MIR583 has a mean methylation status around 10% in the liver, while mean methylation status of the same oligonucleotide sequence in blood and other tissue averages around 95%.

FIG. 5 provides an exemplary boxplot of liver specific methylation patterns that are conserved in healthy and various disease states (obesity, simple steatosis and NASH).

### DETAILED DESCRIPTION

The following detailed description refers to the description below that illustrates exemplary aspects described herein. However, the scope of the aspects described herein are not limited to these aspects but is instead defined by the appended claims. Thus, aspects beyond those described below, such as modified versions of the illustrated aspects, may nevertheless be encompassed by the present description.

While there have been significant improvements in treating cancers, early detection offers patients the best opportunity for early treatment and complete remission. Diagnostic tools such as tissue biopsy are often performed after cancer is initiated or has spread. Treatment at this stage can be difficult and offers poor prognosis for five-year survival. Thus, early detection strategies that are easy to access and low cost are needed.

Exosomes are small extracellular vesicles known for intercellular communication and contain biological material such as proteins, DNA and methyl fragments. They are involved in processes such as inflammatory responses, apoptosis and metastasis.⁹⁻¹¹ Exosomes are present in the bloodstream during both healthy and diseased states, and concentrations can exceed 10⁹ vesicles per mL of blood, they can be used to assess disease state based on tissue of origin.^{10,11}

One mechanism of epigenetic modifications is through DNA methylation, in which DNA methyltransferase (DNMT) adds a methyl group to cysteine residues to regulate gene expression. DNMT on the genome are often higher in HCC patients than in healthy patients. DNMT is involved in silencing tumor suppressors or activating oncogenes which facilitates HCC metastasis, invasion and proliferation.¹³⁻¹⁷ In fact, methylation patterns have been shown to exhibit both tissue-specificity and cancer-specificity in a superior manner than transcriptional expression, genes or proteins.^{7,12,1,19}

Exosomal DNA methylation exhibits diagnostic potential. In one study, scientists analyzed frequently hypermethylation promotor regions associated with pancreatic cancer. They found that methylated patterns in exosomes are representative of similar methylation patterns from a cell of origin in the source tissue of the exosome. The correlation between methylated DNA in exosomes and methylated DNA directly from pancreas showed a 94% correlation factor, thus methylation patterns in exosomal in circulation have the potential for clinical utility to assess disease state.²⁰ Exosomes are newly emerging as diagnostic tools for monitoring cancer dynamics based upon the contents found within.

In addition to exosomes, cell free DNA has also shown diagnostic utility in whole blood using methylated targets to determine disease state. Lehmann-Werman et al (2016) has demonstrated proof of concept assessment of cell death targeting cell free DNA based on methylated tissue specific promotor regions for β-Cell-derived DNA in the circulation of traumatic brain injury patients. Methylation status of patients with traumatic brain injury showed an increase in target methylated regions in circulation as compared to controls. For example, patients within 24 hours post traumatic brain injury showed on average more than 800 copies per mL of β-Cell-derived DNA. Control groups over time showed a steady decline in the copies of targeted methylated regions.²¹ In another example, Lehmann-Werman et al (2016) assessed the promotor methylation status of oligodendrocyte in the serum of remitting and relapsing multiple sclerosis patients to demonstrate that this technique can be used to monitor disease state in stable compared to remitting patients. In this experiment, it was reported that patients with stable disease did not exceed greater than 400 copies per mL for targeted methylated fragments, while relapsing patients demonstrated up to 1,600 copies per mL in target methylated fragmented in circulation.

Methylation of oligonucleotides (e.g., addition of methyl groups to cysteine residues) in non-coding regions of genomic DNA (e.g., upstream or downstream regulatory regions) is one mechanism used to turn on or turn off expression of genes leading to increasing or decreasing levels of proteins encoded by the genes. Thus, identification of methylation patterns in such regulatory regions are an indication of whether or not genes controlled by the regulatory regions are turned on or off. The “on/off” state of genes implicated in cancer (e.g., liver cancer) or liver disease is an early indication of whether or not a subject has an elevated risk of developing cancer or another disease.

In one aspect, the term “methylation pattern” or “beta value” refers to the log ratio of percent methylation of a polynucleotide which is reported on a scale between 0-100%. Beta values greater than 50% indicate a methylated state (and the genomic region is silenced), while beta values less than 50% indicates that the value is unmethylated (and the genomic region is activated).

Methylation patterns or signatures in regulatory regions of genes expressed in tissue samples from liver cancer patients or liver disease patients and control samples from patients without liver cancer and liver disease were analyzed to identify methylation signatures associated with hepatocellular carcinoma (HCC), and liver specific patterns (pre-liver cancer). Patterns were also analyzed to differentiate simple steatosis (benign liver disease) from nonalcoholic steatohepatitis (NASH). Methylation patterns of these signatures can be assessed to determine if the methylation pattern concentration in a liquid biopsy (e.g., blood) is greater than a threshold associated with, for example, increased risk of HCC or early stages of liver cancer or liver disease. Then, the concentration of these methylation patterns can be quantified in patient samples (e.g., liquid biopsy of patient blood or urine).

Liver disease is one of the most common chronic diseases in the US, affecting 1 in 3 Americans, or 100 million people in the US. Liver disease is associated with diabetes, obesity and metabolic syndrome among several other risk factors. While 80 million patients have simple steatosis (the benign form of liver disease), 20 million patients have nonalcoholic steatohepatitis or NASH (the advanced form) that can lead to end stage liver disease, liver related mortality and liver cancer.³¹⁻³⁶ There are no effective noninvasive diagnostic blood-based tools for NASH. In addition, neither imaging nor previously available biomarkers(s) can adequately differentiate benign from advanced liver disease. The sole diagnostic tool is a liver biopsy. Liver biopsies are expensive, invasive and prone to sampling error, and therefore ineffective to serve the population at large.

In addition to these challenges, liver disease is typically asymptomatic until patients have progressed to advanced stages or liver cancer. At this stage, liver transplants are the most effective treatment. Without a reliable and non-invasive test, most patients are diagnosed at late stages, when outcomes are poor, mortality rates high, and healthcare spending can cost up to $1,000,000 per patient.³¹⁻³⁶

When diagnosing liver disease, two diagnostic assessments can be considered. First, the degree of fibrosis (liver scarring) must be considered because fibrosis accelerates liver related mortality. There are 4 stages of fibrosis, and stages greater than fibrosis stage 2 (F2) is a critical point in the progression from NAFLD to NASH, and the risk of liver specific mortality has been shown to increase 50-80% after F2.³¹⁻³⁵

The second diagnostic assessment is the differentiation of simple steatosis (benign) from NASH (advanced liver disease) can be considered. In some instances, detection of fibrosis and NASH can be used to obtain an accurate and early diagnosis of liver disease.

Liver biopsies are partially effective at diagnosing liver disease (e.g., 89% sensitivity at 90% specificity). However, they are expensive ($2,500 per test), invasive, prone to sample errors, and cause bleeding. Imaging techniques, such as CT scans, ultrasounds or transient elastography are capable of staging fibrosis, but there are several limitations. For example, these imaging tools cannot be used if there is significant fat or fluid between the chest wall and the liver and are associated with failed results in nearly 20% of patients, particularly those with obesity.³²

In addition to these challenges, 40% of patients with NASH do not have any underlying fibrosis, and many imaging tools cannot diagnose NASH in the absence of fibrosis. Finally, there are more than 2 dozen serum markers with modest sensitivity and specificity ranging from 52%-79% sensitivity at 85% specificity for staging fibrosis. However, current serum tests are not capable of differentiating benign disease from NASH.³¹⁻³⁵

Aspects described herein are more accurate than the alternatives described above, less expensive than the gold standard (liver biopsy) and are capable of staging fibrosis and differentiating simple steatosis (benign) from NASH (advanced liver disease).

Aspects described herein target both liver specific and NASH specific methylation signatures. By targeting liver specific methylation signatures in circulation, an indirect representation of liver fibrosis (liver scarring) can be obtained. Without being bound by theory, it is believed that excess liver-derived methylation patterns are associated with varying degrees of liver fibrosis. In some instances, the degree of liver specific methylation signatures, as described herein, can provide a sequential assessment to determine no fibrosis (F0) or mild fibrosis (F1), significant fibrosis (F≥2), advanced fibrosis (F≥3) from cirrhosis (F=4). For example, in some instances, less than 25 copies per mL of liver specific methylation signature sequences in a liquid biopsy (e.g., blood sample) represents a patient phenotype with mild fibrosis, while a patient with more than 25 copies per mL of liver specific methylation signature sequences in a liquid biopsy would represent a patient phenotype with significant fibrosis (F2-F4).

Aspects described herein also assess NASH specific methylation signatures that are present in patients with NASH and can differentiate simple steatosis from NASH. The NASH specific methylation signatures indicate that the patient has progressed to the advanced form of liver disease.

Aspects described herein provide a first method of determining if an HCC specific methylation signature is detected in a subject suspected of having liver cancer by (a) obtaining a tissue sample comprising a nucleic acid from the subject; (b) isolating the nucleic acid from the tissue sample; (c) determining a concentration of the HCC specific methylation signature in the tissue sample by contacting the nucleic acid with at least one HCC specific methylation signature detection molecule; and (d) determining if the HCC specific methylation signature is detected in the tissue sample.

As used herein throughout, an HCC specific methylation signature detection molecule is a probe that is capable of binding to one of the nucleotide sequences in Table 1 below, i.e., one of the nucleotide sequences of SEQ ID NOS:1-100. In some embodiments, the probe is at least 5, at least 6, at least 7, at least 8, at least 9, at least 10, at least 15, at least 20, 5-10, 10-15, 15-20, 20-25, 25-30, 30-40, 40-50, 50-70, 70-100, or any specific number or ranges of nucleotides derived therefrom capable of binding to one of the nucleotide sequences in Table 1 below, i.e., one of the nucleotide sequences in SEQ ID NOS:1-100. In some embodiments, at least 1, at least 2, at least 3, at least 4, at least 5, 1-10, 1-15, 1-20, 1-25, 1-30, 1-40, 1-50, 1-60, 1-70, 1-80, 1-90, 1-100 detections molecules or any specific number of detection molecules or ranges derived therefrom can be used in the methods disclosed herein.

The term “HCC specific methylation signature” refers to a methylation signature associated with risk of having hepatocellular carcinoma (HCC) as described herein.

In some instances, the concentration of the methylated signatures provided in Table 1 and associated with the indicated genes can be quantified in circulation by measuring the copy number of the methylated signature per milliliter (mL) in a tissue sample.

The term “tissue sample” refers to, for example, a sample of tissue that contains a cell, cell fragment, exosome, or other component from a tissue of origin of interest. For example, a tissue sample can include a blood sample containing a cell, an exosome, or cell free (cf) DNA. The tissue of origin can be an organ or system of interest (e.g., liver, spleen, kidney, bile duct, breast, prostate, lymph node, etc.). A solid or semi-solid tissue sample can be processed and liquefied or can already be in liquid form (e.g., blood). The term “tissue sample” also refers to a portion of bodily fluid (e.g., peripheral blood, urine, or saliva) or tissue (i.e., that can be homogenized) that can be obtain from a subject through any suitable means of collection.

The term “tissue of origin” refers to the tissue or organ a component was originally formed or where the component metastasized to after originating in a different tissue or organ (e.g., liver, lung, pancreas, intestine, spleen, prostate, or cardiovascular). In some instances, the tissue of origin can be the liver.

The term “control tissue sample” refers to a tissue sample having a tissue of origin similar to the tissue of origin from a subject who does not have the disease or condition being evaluated. For example, a test tissue sample can have liver as a tissue of origin (e.g., liver tissue, or an exosome, cell, or cfDNA originally from the liver) and be derived from a subject diagnosed with liver cancer, suspected of having liver cancer, or being tested to determine whether the subject has liver cancer. The corresponding control tissue sample can have liver as a tissue of origin and be derived from a subject who does not have liver cancer. In some instances, the control tissue can be derived from the same subject being tested at a point in time when the subject was confirmed not to have liver cancer.

The term “exosome” refers to extracellular vesicles of endosomal origin and produced by eukaryotic cells. The term “methylation pattern” refers to the pattern of epigenetic modification by which DNA methyltransferase (DNMT) adds a methyl group to cysteine residues in a nucleic acid molecule. DNA methylation is one mechanism for modifying gene expression and is associated with a variety of cell phenotypes. The dysregulation of DMNTs are higher in HCC patients than normal patient hepatocytes and involved in silencing tumor suppressors that facilitates HCC metastasis, invasion and proliferation.³⁶

The “pattern” or “signature” of methylation can refer to the number and order of cysteine residues having attached methyl groups. A methylation pattern can include identifying the degree to which (e.g., the percentage of cysteine residues) a nucleic acid has cysteine residues with methyl groups.

The term “associated with” refers to a methylation pattern that within between 0 base pairs to about 3 kilobases and is either upstream or downstream from the closest gene. The term “associated with,” as used herein, does not require regulating the closest gene body or having a functional consequence on the closest gene. The term “associated with,” as used herein, can refer to the nearest gene of interest in base pair numbers. The methylation signature can be located, for example, in an intragenic region, in an intron, in an exon, in a promoter region, or in a 3′ or 5′ untranslated region.

The term “liver specific methylation signature” refers to a methylation signature associated with a region associated with the liver and related to any stage of liver disease. In some instances, the liver specific methylation signature is associated with a gene related to liver tissue and not with genes associated with other tissues or organs in the body.

The liver specific methylation signature can be present during a healthy state, and the liver specific methylation pattern (or beta value) can be conserved during varying stages of disease. For example, if a liver specific methylation signature has a beta value of 0.15 on a healthy liver, the liver specific methylation signature can have a beta value within 5% of this value during various degrees of fibrosis (such as fibrosis stage 2). In this example, the liver specific methylation signature can have a beta value of 0.18 during this diseased state.

A “conserved” methylation signature or beta value is defined as having less than a 5% difference during any state (i.e., healthy, diseased, cancerous state) directly on the tissue of original (for example the liver).

The term “degree of methylation” refers to a measurement of the percentage of cytosine residues in a nucleic acid backbone that have added methyl groups.

The term “control cell” refers to a cell that does not exhibit a tumorigenic phenotype. For example, with respect to liver cells, a normal cell can include a non-tumorigenic normal cell and a non-tumorigenic cirrhotic cell. In further aspects, the degree of methylation of the nucleic acid can be determined by measuring the beta value or methylation status of a component of a cell contained in an exosome or circulating material (such as cell free material). The beta value or methylation status can be compared to a control normal cell, and a chronic cirrhosis cell. For example, the degree of methylation can be determined using bisulfite sequencing (e.g., post-bisulfate adapter-tagging (PBAT)), targeted methylation sequencing (targeted bisulfite sequencing or methyl sequencing), pyrosequencing, methylation arrays, digital droplet PCR and/or methylation specific PCR.

The term “circulating material” refers to biological material that can be found, for example, in blood, serum, urine or tissue and collected from, for example, a blood, serum, urine or tissue sample.

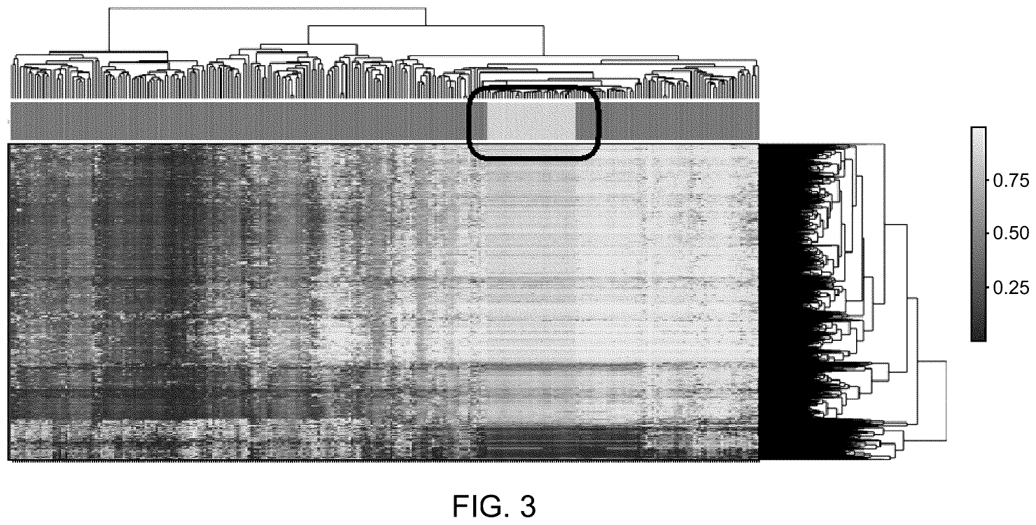

In some instances of the first method, the concentration of the HCC specific methylation signature exceeds about 25 copies per ml in the tissue sample. Without being bound by theory, it is believed that, in some instances, a concentration threshold of exceeding 25 copies per mL of one or more HCC methylation signature is indicative of a subject having liver cancer because the clustering regions, as shown, for example, in FIG. 3 and Table 1, are present in tissue samples from a subject having HCC and not present in a tissue sample from a subject who does not have HCC (e.g., a control tissue sample).

In some instances of the first method, the tissue sample is selected from the group consisting of blood, urine, stool, liver, and lymph.

In some instances of the first method, the subject at high risk for liver cancer is selected from the group consisting of a subject having viral hepatitis, obesity, diabetes, polycystic ovary syndrome, metabolic syndrome, non-alcoholic fatty liver disease (NAFLD), fibrosis, cirrhosis and/or other forms of chronic liver disease.

In some instances of the first method, the tissue sample is from a subject who has not been diagnosed with liver cancer.

In some instances of the first method, the HCC specific methylation signature comprises at least 2 to 7 CpG marker sites.

In some instances, the first method further comprises extracting circulating material from the tissue sample prior to step (b).

In some instances of the first method, the circulating material is extracted from the tissue sample by exosome isolation or cell free DNA isolation.

In some instances of the first method, at least one HCC specific methylation signature is associated with one or more of POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

Further aspects provide a second method of determining a difference in a beta value between an HCC specific methylation signature in a tissue sample and a control tissue sample by determining a beta value of the HCC specific methylation signature in a nucleic acid from the tissue sample, determining a beta value of the HCC specific methylation signature in a nucleic acid from the control tissue sample, and determining if a difference in an average beta value between the beta value of the HCC specific methylation signature in the tissue sample and the beta value of the HCC specific methylation signature in the control tissue sample is greater than or equal to 10%.

The term “average beta” refers to the log ratio of percent methylation which is reported on a scale between 0-100%. The term “determining the beta value” for example of an HCC specific methylation signature refers to use of DNA methylation probes designed to bind to and quantify the degree of methylation of a specific sequence.

In some instances of the second method, the control tissue sample is from a subject at high risk for liver cancer but has not been diagnosed with liver cancer.

In some instances of the second method, the HCC specific methylation signature is associated with one or more of POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

Aspects described herein provide a third method of determining if a liver specific methylation signature is detected in a subject suspected of having liver cancer, by (a) obtaining a tissue sample comprising a nucleic acid from the subject; (b) isolating the nucleic acid from the tissue sample; (c) determining a concentration of the liver specific methylation signature in the tissue sample by contacting the nucleic acid with at least one liver specific methylation signature detection molecule; and (d) determining if the liver specific methylation signature is detected in the tissue sample.

As used herein throughout, a liver specific methylation signature detection molecule is a probe that is capable of binding to one of the nucleotide sequences in Table 2 below, i.e., one of the nucleotide sequences in SEQ ID NOS:101-255. In some embodiments, the probe is at least 5, at least 6, at least 7, at least 8, at least 9, at least 10, at least 15, at least 20, 5-10, 10-15, 15-20, 20-25, 25-30, 30-40, 40-50, 50-70, 70-100, 100-150, 150-200, 200-255, or any specific number or ranges of nucleotides derived therefrom capable of binding to one of the nucleotide sequences in Table 2 below, i.e., one of the nucleotide sequences in SEQ ID NOS:101-255. In some embodiments, at least 1, at least 2, at least 3, at least 4, at least 5, 1-10, 1-15, 1-20, 1-25, 1-30, 1-40, 1-50, 1-60, 1-70, 1-80, 1-90, 1-100, 1-150, 1-200, 1-250, or 255 detections molecules or any specific number of detection molecules or ranges derived therefrom can be used in the methods disclosed herein.

In some instances of the third method, the concentration of the HCC specific methylation signature concentration exceeds about 25 copies per ml in the tissue sample.

In some instances of the third method, the tissue sample is selected from the group consisting of blood, urine, stool, liver, and lymph.

In some instances of the third method, the subject at high risk for liver cancer is selected from the group consisting of a subject having viral hepatitis, obesity, diabetes, polycystic ovary syndrome, metabolic syndrome, non-alcoholic fatty liver disease (NAFLD), fibrosis, cirrhosis and/or other forms of chronic liver disease.

In some instances of the third method, the tissue sample is from a subject who has not been diagnosed with liver cancer.

In some instances of the third method, the liver specific methylation signature comprises at least 2 to 7 CpG marker sites.

In some instances, the third method further comprises extracting circulating material from the tissue sample prior to step (b).

In some instances of the third method, the circulating material is extracted from the tissue sample by exosome isolation or cell free DNA isolation.

In some instances of the third method, the at least one liver specific methylation signature is associated with one or more of AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TAC1, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

Aspects described herein provide a fourth method of determining a difference in a conserved beta value for a liver specific methylation signature by determining a beta value of the liver specific methylation signature in a nucleic acid from a tissue sample, determining a beta value of the liver specific methylation signature in a nucleic acid from a control tissue sample, and determining if a difference in the conserved beta value between the beta value of the liver specific methylation signature in the tissue sample and the beta value of the liver specific methylation signature in the control tissue sample is less than or equal to 5%.

In some instances of the fourth method, the control tissue sample is from a subject at high risk for liver cancer, but has not been diagnosed with liver cancer.

In some instances of the fourth method, the liver specific methylation signature is associated with one or more of AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TAC1, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

Aspects described herein provide a fifth method of treating liver cancer in a subject suspected of having liver cancer, by (a) obtaining a tissue sample comprising a nucleic acid from the subject, (b) isolating the nucleic acid from the tissue sample, (c) determining a concentration of an HCC specific methylation signature in the tissue sample by contacting the nucleic acid with at least one HCC specific methylation signature detection molecule, and (d) treating the subject for liver cancer if the HCC specific methylation signature is detected in the tissue sample.

In some instances of the fifth method, the concentration of the HCC specific methylation signature concentration exceeds about 25 copies per ml in the tissue sample.

In some instances of the fifth method, the tissue sample is selected from the group consisting of blood, urine, stool, liver, and lymph.

In some instances of the fifth method, treating the subject for liver cancer comprises administering a drug selected from the group consisting of one or more of sorafenib, lenvatinib, regorafenib, cabozantinib, nivolumab, pembrolizumab, and ramucirumab to the subject.

In some instances of the fifth method, the tissue sample is from a subject at high risk for liver cancer but has not been diagnosed with liver cancer.

In some instances of the fifth method, the subject at high risk for liver cancer is selected from the group consisting of a subject having viral hepatitis, obesity, diabetes, polycystic ovary syndrome, metabolic syndrome, non-alcoholic fatty liver disease (NAFLD), fibrosis, cirrhosis and chronic liver disease.

In some instances of the fifth method, the tissue sample is from a subject who has not been diagnosed with liver cancer.

In some instances of the fifth method, the HCC specific methylation signature comprises at least 2 to 7 CpG marker sites.

In some instances, the fifth method further comprising extracting circulating material from the tissue sample prior to step (b).

In some instances of the fifth method, the circulating material is extracted from the tissue sample by exosome isolation or cell free DNA isolation.

In some instances of the fifth method, the HCC specific methylation signature detection molecule is capable of binding to the HCC specific methylation signature, wherein the HCC specific methylation signature is associated with one or more of POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

Aspects described herein provide a sixth method of treating liver cancer in a subject suspected of having liver cancer by determining a concentration of an HCC specific methylation signature in a nucleic acid from a tissue sample, determining a concentration of an HCC specific methylation signature in a nucleic acid from a control tissue sample, determining a beta value of an HCC specific methylation signature in a nucleic acid from a tissue sample, determining a beta value of the HCC specific methylation signature in a nucleic acid from a control tissue sample; and treating the subject for liver cancer if the concentration of the HCC specific methylation signature in the nucleic acid from the tissue sample exceeds the concentration of the HCC specific methylation signature in the nucleic acid from the control tissue sample, and the beta value of the HCC specific methylation signature in a nucleic acid from the tissue sample is 10% or greater than the beta value of the HCC specific methylation signature in a nucleic acid from the control tissue sample.

In some instances of the sixth method, the control tissue sample is from a subject at high risk for liver cancer but has not been diagnosed with liver cancer

Further aspects described herein provide a seventh method of treating liver cancer in a subject suspected of having liver cancer, by (a) obtaining a tissue sample comprising a nucleic acid from the subject, (b) isolating the nucleic acid from the tissue sample, (c) determining a concentration of a liver specific methylation signature in the tissue sample by contacting the nucleic acid with at least one liver specific methylation signature detection molecule, (d) determining a concentration of a HCC specific methylation signature in the tissue sample by contacting the nucleic acid with at least one HCC specific methylation signature detection molecule; and (e) treating the subject for liver cancer if the liver specific methylation signature is detected in the tissue sample and the HCC specific methylation signature is detected in the tissue sample.

In some instances of the seventh method, the tissue sample is from a subject at high risk for liver cancer but has not been diagnosed with liver cancer.

In some instances of the seventh method, the tissue sample is from a subject who has not been diagnosed with liver cancer.

In some instances of the seventh method, the tissue sample is selected from the group consisting of blood, urine, stool, liver, and lymph.

In some instances of the seventh method, treating the subject for liver cancer comprises administering a drug selected from the group consisting of one or more of sorafenib, lenvatinib, regorafenib, cabozantinib, nivolumab, pembrolizumab, and ramucirumab to the subject.

In some instances of the seventh method, the liver specific methylation signature comprises at least 2 to 7 CpG marker sites.

In some instances, the seventh method further comprises extracting circulating material from the tissue sample prior to step (b).

In some instances of the seventh method, the circulating material is extracted from the tissue sample by exosome isolation or cell free DNA isolation.

In some instances of the seventh method, the liver specific methylation signature detection molecule is selected from the group consisting of one or more detection molecules capable of binding to the liver specific methylation signature, wherein the liver specific methylation signature is associated with one or more of AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TAC1, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

In some instances of the seventh method, the HCC specific methylation signature detection molecule is selected from the group consisting of one or more detection molecules capable of binding to the HCC specific methylation signature, wherein the HCC specific methylation signature is associated with one or more of POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

Aspects described herein provide an eighth method of treating liver cancer in a subject suspected of having liver cancer by determining a concentration of an HCC specific methylation signature in a nucleic acid from a tissue sample; determining a concentration of an HCC specific methylation signature in a nucleic acid from a control tissue sample; determining a concentration of an liver specific methylation signature in a nucleic acid from a tissue sample; determining a concentration of an liver specific methylation signature in a nucleic acid from a control tissue sample; determining a beta value of an HCC specific methylation signature in a nucleic acid from a tissue sample; determining a beta value of the HCC specific methylation signature in a nucleic acid from a control tissue sample; determining a beta value of a liver specific methylation signature in a nucleic acid from a tissue sample; determining a beta value of the liver specific methylation signature in a nucleic acid from a control tissue sample; and treating the subject for liver cancer if the concentration of the HCC specific methylation signature in the nucleic acid from the tissue sample exceeds the concentration of the HCC specific methylation signature in the nucleic acid from the control tissue sample, the concentration of liver specific methylation signature in the nucleic acid from the tissue sample exceeds the concentration of the liver specific methylation signature in the control tissue sample, the beta value of the HCC specific methylation signature in a nucleic acid from the tissue sample is 10% or greater than the beta value of the HCC specific methylation signature in a nucleic acid from the control tissue sample, and the beta value of the liver specific methylation signature in a nucleic acid from the tissue sample is 10% or greater than the beta value of the liver specific methylation signature in a nucleic acid from the control tissue sample.

In some instances of the eighth method, the control tissue sample is from a subject at high risk for liver cancer but has not been diagnosed with liver cancer.

Further aspects described herein provide a first kit comprising at least one liver specific methylation signature detection molecule capable of binding to one or more methylation patterns associated with AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TACT, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

Aspects described herein provide a second kit comprising at least one HCC specific methylation signature detection molecule capable of binding to one or more methylation patterns associated with POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

Aspects described herein provide a ninth method of detecting at least one HCC specific methylation signature in a tissue sample of a subject by contacting a HCC specific methylation signature detection molecule with nucleic acid obtained from the tissue sample, wherein the HCC specific methylation signature detection molecule is capable of binding to one or more methylation patterns associated with POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1.

In some instances of the ninth method, the tissue is selected from the group consisting of blood, stool, urine, liver, and lymph.

Further aspects provide a tenth method of detecting at least one liver specific methylation signature in a tissue sample of a subject by contacting a liver specific methylation signature detection molecule with nucleic acid obtained from the tissue sample, wherein the liver specific methylation signature detection molecule is capable of binding to one or more methylation patterns associated with AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TAC1, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

In some instances of the tenth method, the tissue sample is selected from the group consisting of blood, stool, urine, liver, and lymph.

Aspects described herein provide an eleventh method of determining if a NASH specific methylation signature is detected in a subject suspected of having NASH by (a) obtaining a tissue sample comprising a nucleic acid from the subject, (b) isolating the nucleic acid from the tissue sample, (c) determining a concentration of the NASH specific methylation signature in the tissue sample by contacting the nucleic acid with a NASH specific methylation signature detection molecule; and (d) determining if the NASH specific methylation signature is detected in the tissue sample.

In some instances of the eleventh method, the concentration of the NASH methylation signature concentration exceeds about 25 copies per ml in the tissue sample.

In some instances of the eleventh method, the tissue sample is from a subject at high risk for liver cancer but has not been diagnosed with liver cancer.

In some instances of the eleventh method, the tissue sample is selected from the group consisting of blood, urine, stool, liver, and lymph.

In some instances of the eleventh method, the subject at high risk for liver cancer is selected from the group consisting of a subject having viral hepatitis, obesity, diabetes, polycystic ovary syndrome, metabolic syndrome, non-alcoholic fatty liver disease (NAFLD), fibrosis, cirrhosis and/or other forms of chronic liver disease.

In some instances of the eleventh method, the tissue sample is from a subject who has not been diagnosed with liver cancer.

In some instances of the eleventh method, the NASH specific methylation signature comprises at least 2 to 7 CpG marker sites.

In some instances, the eleventh method further comprises extracting circulating material from the tissue sample prior to step (b).

In some instances of the eleventh method, the circulating material is extracted from the tissue sample by exosome isolation or cell free DNA isolation.

In some instances of the eleventh method, the NASH specific methylation signature detection molecule is selected from the group consisting of one or more detection molecules capable of binding to at least one of ARHGEF25, NEU4, PCOLCE, CREB5, CASP8, FMN1, ADGRG1, AQP1, CD74, and CAVIN2.

As used herein throughout, a NASH specific methylation signature detection molecule is a probe that is capable of binding to one of the nucleotide sequences associated with the genes in Table 3 below, e.g., SEQ ID NO:164. In some embodiments, the probe is at least 5, at least 6, at least 7, at least 8, at least 9, at least 10, at least 15, at least 20, 5-10, 10-15, 15-20, 20-25, 25-30, 30-40, 40-50, 50-70, 70-100, 100-150, 150-200, 200-255, or any specific number or ranges of nucleotides derived therefrom capable of binding to one of the nucleotide sequences associated with the genes in Table 3 below, e.g., SEQ ID NO:164. In some embodiments, at least 1, at least 2, at least 3, at least 4, at least 5, 1-10, or more detections molecules or any specific number of detection molecules or ranges derived therefrom can be used in the methods disclosed herein.

Further aspects provide a twelfth method of determining a difference in a beta value between a NASH specific methylation signature in a tissue sample and a control tissue sample by determining a beta value of the NASH specific methylation signature in a nucleic acid from the tissue sample, determining a beta value of the NASH specific methylation signature in a nucleic acid from the control tissue sample; and determining if a difference in an average beta value between the beta value of the NASH specific methylation signature in the tissue sample and the beta value of the NASH specific methylation signature in the control tissue sample is greater than or equal to 10%.

In some instances of the twelfth method, the tissue sample is from a subject at high risk for NASH but has not been diagnosed with NASH.

Aspects described herein provide a thirteenth method of determining if a liver specific methylation signature is detected in a subject suspected of having fibrosis by (a) obtaining a tissue sample comprising a nucleic acid from the subject, (b) isolating the nucleic acid from the tissue sample, (c) determining a concentration of a liver specific methylation signature in the tissue sample by contacting the nucleic acid with a liver specific methylation signature detection molecule, and (d) determining if the liver specific methylation signature is detected in the tissue sample.

In some instances of the thirteenth method, the concentration of the liver methylation signature concentration exceeds about 25 copies per ml in the tissue sample.

In some instances of the thirteenth method, the tissue sample is selected from the group consisting of blood, urine, stool, liver, and lymph.

In some instances of the thirteenth method, the subject at high risk for fibrosis is selected from the group consisting of a subject having viral hepatitis, obesity, diabetes, polycystic ovary syndrome, metabolic syndrome, non-alcoholic fatty liver disease (NAFLD) and/or chronic liver disease.

In some instances of the thirteenth method, the tissue sample is from a subject who has not been diagnosed with fibrosis.

In some instances of the thirteenth method, the liver specific methylation signature comprises at least 2 to 7 CpG marker sites.

In some instances, the thirteenth method further comprises extracting circulating material from the tissue sample prior to step (b).

In some instances of the thirteenth method, the circulating material is extracted from the tissue sample by exosome isolation or cell free DNA isolation.

In some instances of the thirteenth method, the liver specific methylation signature detection molecule is capable of binding to a liver specific methylation signature associated with one or more of AMY1C, GSTM1, NOTCH2, NBPF26, PKLR, PKLR, PKLR, PKLR, MIR3675, MIR3675, ESPNP, ESPNP, ESPNP, MIR4677, CHST15, LOC441666, DNAJC12, SNORD131, SNORD131, CAPRIN1, LOC692247, LOC692247, LOC692247, LOC692247, FAM86C2P, C12orf75, FAM230C, OR11H12, GOLGA8F, LOC100288203, KLF13, KLF13, LOC101928042, GOLGA6A, PDXDC1, MYH11, PDPK1, LOC652276, FLJ42627, CCNYL3, LINC02167, SERPINF2, KCNJ18, KCNJ18, KCNJ18, KCNJ18, UBBP4, UBBP4, UBBP4, CCL16, SRCIN1, SRCIN1, SRCIN1, SRCIN1, SRCIN1, LOC653653, SBNO2, INSR, CD320, MIR4436B2, PRSS40A, ACVR2A, MIR5702, NEU4, CYTOR, MIR1-1HG, MIR1-1HG, CBS, LINC00319, FTCD-AS1, FTCD-AS1, LOC102724159, RNA28SN2, FRG1FP, FRG1FP, CCT8L2, CCT8L2, CCT8L2, CCT8L2, CCT8L2, CCT8L2, RIMBP3, RIMBP3, HIC2, CYP2D7, LINC02012, BDH1, BDH1, BDH1, BDH1, BDH1, BDH1, BDH1, BDH1, BDH1, BDH1, BDH1, BDH1, ZNF860, LOC100130207, GRM7-AS3, FAM241A, GK3P, UGT2B4, MIR548P, HCN1, HCN1, HCN1, LOC102467080, SMA4, C5orf49, NBPF22P, MIR583, CD24, ERMARD, LOC100131532, LOC100131532, UBD, HCG4B, HCG9, TRIM15, TRIM15, TRIM15, HSPA1B, B3GALT4, MLIP, GUSBP4, FHL5, CUL1, ZNF716, LOC100287704, LOC100287834, GTF2IRD2, GTF2IRD2B, GTF2IP1, GTF2IP1, PCLO, TAC1, MIR12119, ZHX2, GSDMD, MROH6, KBTBD11-OT1, ERICH1, USP17L8, GPR21, CNTNAP3, FAM242F, FAM242F, CNTNAP3B, RIC1, LOC100996643, ZNF658, FAM74A3, and/or LOC102723709.

Aspects described herein provide a fourteenth method of determining a difference in a conserved beta value for a liver specific methylation signature by determining a beta value of the liver specific methylation signature in a nucleic acid from a tissue sample of a subject suspected of having fibrosis, determining a beta value of the liver specific methylation signature in a nucleic acid from a control tissue sample, and determining if a difference in the conserved beta value between the beta value of the liver specific methylation signature in the tissue sample and the beta value of the liver specific methylation signature in the control tissue sample is less than or equal to 5%.

In some instances of the fourteenth method, the tissue sample is from a subject at high risk for fibrosis but has not been diagnosed with fibrosis.

Without being bound by theory, it is believed that excess liver specific methylation signatures are indirectly associated with various forms of excess hepatic debris released into the bloodstream, urine, stool and saliva among various other tissues. For example, during a healthy state the liver specific methylation signature has a defined range of liver specific methylation signatures per concentration of blood. For example, during a diseased state, the liver specific methylation signature can be associated with varying stages of fibrosis (mild, significant or advanced), progressed disease, excess necrosis, apoptosis and/or higher risk of development disease.

Without being bound by theory, it is believed that detection of a methylation signature (e.g., HCC specific, liver specific) in a tissue sample from a subject can indicate the subject has or has an elevated risk of developing a disease (e.g., HCC, simple steatosis, NASH, fibrosis).

FIG. 3 provides an exemplary HCC Epigenotype Heatmap and Random Forest Machine Learning Output. Differentially methylated blocks (200 bp in size) are shown clustered in the squared area indicating these methylated HCC specific signatures are found in subjects having HCC and not found in subjects with normal tissue.

Thus, methylation signatures associated with the indicated associated genes can be used to distinguish a subject with a disease or a high risk of developing a disease from a normal subject. In some instances, detecting 25 copies/mL of blood of an HCC specific or liver specific methylation signature is indicative a subject having or at high risk for the disease (e.g., HCC, liver disease, or fibrosis) associated with the methylation signature.

Table 1 provides exemplary HCC specific methylation signatures (last column) associated with the indicated gene (first column). Table 2 provides exemplary liver specific methylation signatures. Table 3 provides exemplary NASH specific methylation signatures.

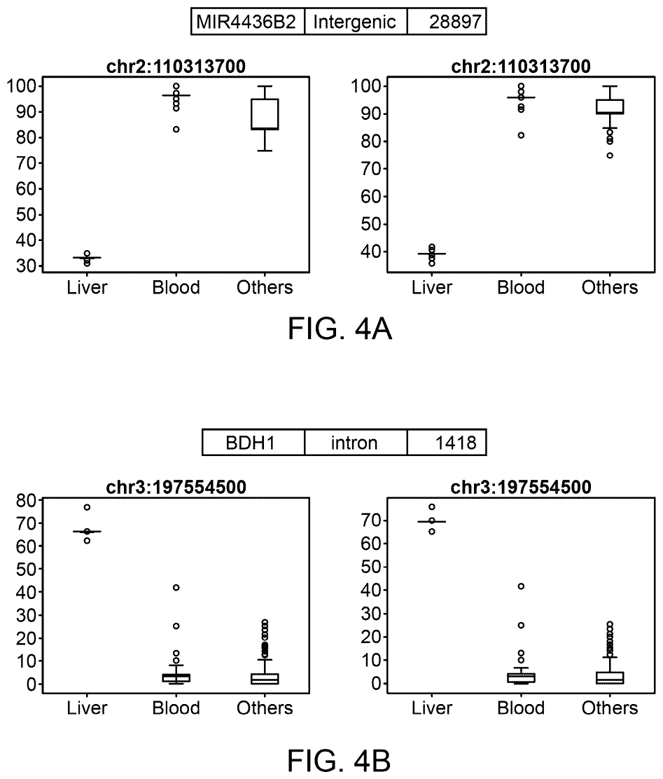

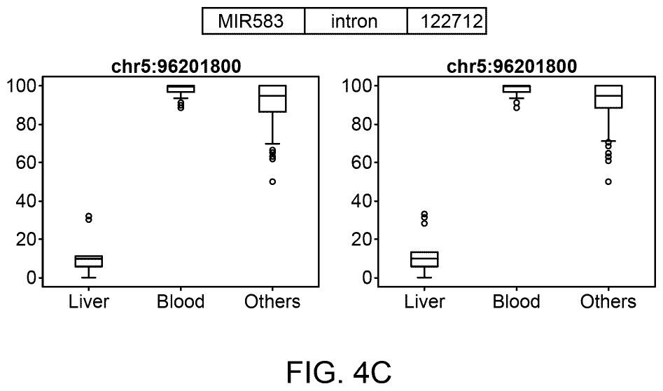

Without being bound by theory, in some instances, liver specific methylation patterns exhibit a specific and unique methylation signature that is not found in other tissues, nor found in healthy human blood. For example, FIG. 4A illustrates that methylation status of oligonucleotide sequence associated with miR443682 has a mean methylation status averages 33% (see left figure), while mean methylation status of same oligonucleotide sequence in blood cells averages 95% and finally mean methylation status of other tissues averages 82%. There methylation status found on the liver has no overlap with any other tissue assessed and is thus defined as a liver specific methylation marker.

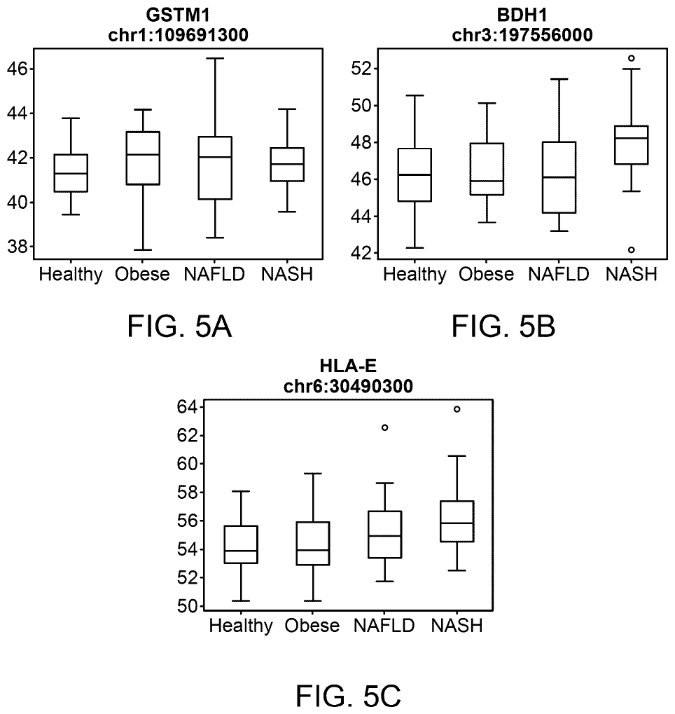

Without being bound by theory, in some instances, a liver specific methylation signature can maintain a similar methylation pattern between varying stages of liver disease, fibrosis, cirrhosis and liver cancer in order to be defined as a “liver specific methylation signature.” For example, FIG. 5 illustrate that the methylation status shown between controls is conserved (less than 5% difference between varying states).

The term “beta value” refers to the log ratio of percent methylation of a polynucleotide which is reported on a scale between 0-100%. Without being bound by theory, it is believed that a change in beta value (e.g., degree of methylation) of a methylation signature between the control sample and the tissue sample of at least 10% beta (e.g., in either direction, hypomethylated or hypermethylation) is associated with liver cancer.

For example, an average beta of greater than or equal to 10% between a tissue sample and a control tissue sample is indicative of liver cancer and the subject can be treated for liver cancer. In this example, past this threshold, the patient no longer has stable liver disease, and the presence of these targets is directly associated with a HCC (liver cancerous) phenotype. In some instances, a beta value that is conserved between control and tissue sample within 5% average beta is indicative of a liver specific methylation pattern.

In some instances, two measurements (e.g., concentration of a methylation signature and beta value (degree of methylation)) can be used to determine if a subject has a disease (e.g., HCC, simple steatosis, NASH, fibrosis). In some instances, a tissue sample from a subject can be assessed for concentration of a methylation signature and degree of methylation of a methylation signature. If both assessments indicate the subject has or is at high risk for a disease (e.g., HCC, liver disease, fibrosis), the subject can be treated for the disease.

Without being bound by theory, it is believed that a beta value that is conserved (e.g., a conserved beta value) between control and tissue sample within 5% average beta is associated with a liver specific methylation signature and can be indicative of high risk of developing fibrosis. In some instances, these liver specific patterns can be used as an indirect measurement of excess liver scaring (i.e., fibrosis).

In some instances, nucleic acid (e.g., deoxyribonucleic acid (DNA) or ribonucleic acid (RNA)) can be isolated or purified from a component by conventional means, or as described herein. In one aspect, the degree of methylation of the nucleic acid can be determined as described herein, or by conventional means, and compared to a control nucleic acid (e.g., a nucleic acid from a component having a normal or non-tumorigenic phenotype). As described herein and in WO2020010311, hereby incorporated by reference in its entirety, the degree of methylation can be used to determine if a cell component has tumorigenic (e.g., precancerous or cancerous) or normal phenotype.

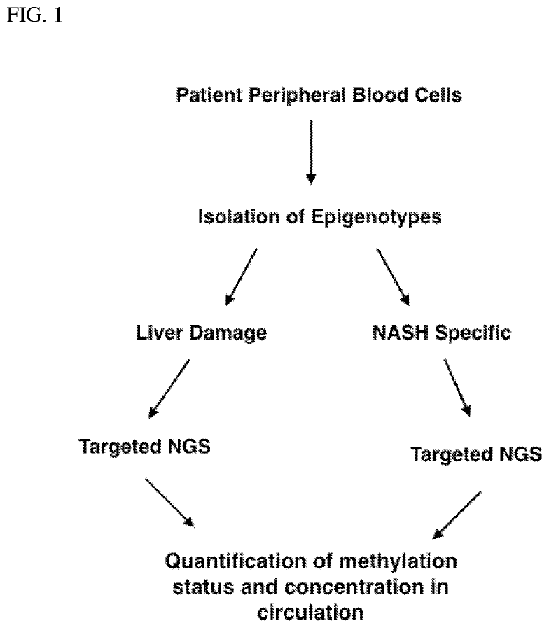

In reference to FIG. 1, a patient sample (e.g., blood sample) can be processed, and epigenotypes can be isolated through exemplary methods (e.g., cell free DNA or exosome isolation methods). Liver-specific methylation signatures and liver-cancer specific methylation signatures can be assessed for both quantification of methylation status and concentration of methylated regions in patient blood through exemplary methods (e.g., targeted methylation sequencing, whole genome bisulfite sequencing or methylation specific PCR). The presence of both liver specific and liver cancer specific methylation signatures concentration can be assessed in copies per mL to quantify epigenotypes in circulation and determine, for example, if the patient currently has liver cancer.

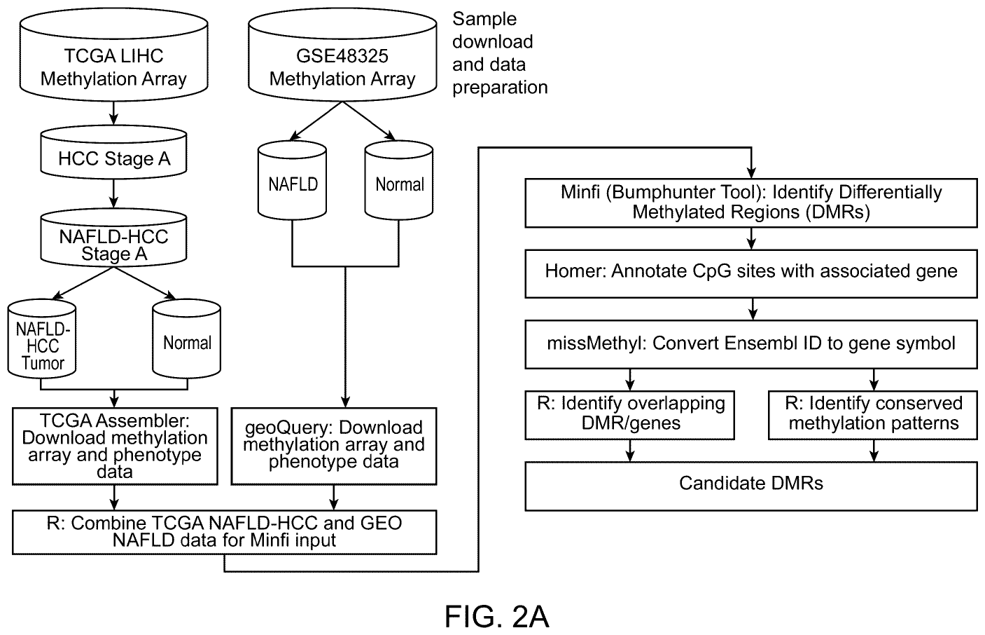

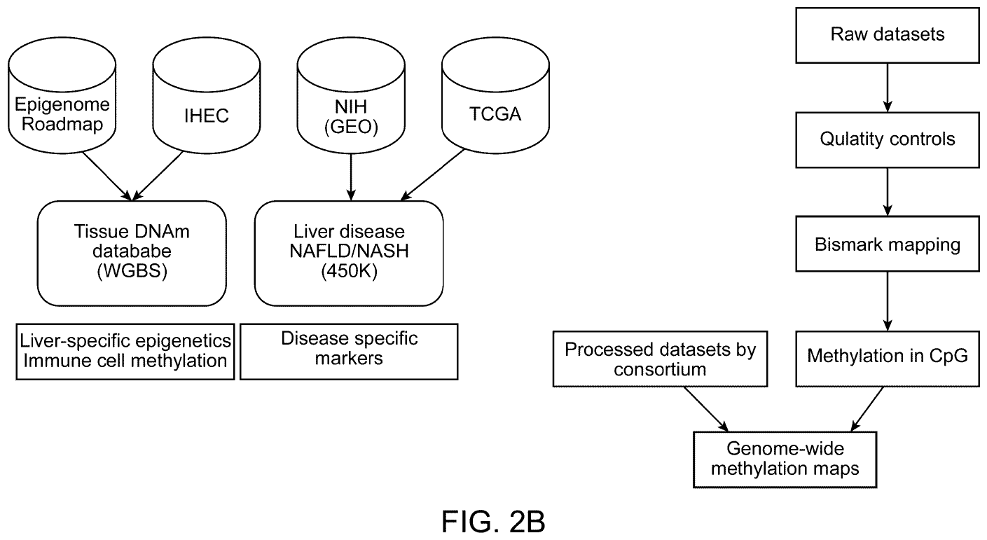

In reference to FIG. 2, samples from patients from TCGA (The Cancer Genome Atlas) Stage I and II HCC and controls, and GSE NAFLD (Non-alcoholic fatty liver disease) and healthy controls can be analyzed using whole genome bisulfite sequencing from the Epigenome Roadmap and International Human Epigenome Consortium (IHEC) and 450 k methylation arrays.^{26,27,39,40}. Each CpG can be associated with measurements: a “methylated” measurement and an “unmethylated” measurement. FIG. 2A: MinFi is a Bioconductor open-source tool than can be used for analyzing Illumina Methylation arrays and the mini Bumphunter tool was used to identify differentially methylated regions within the dataset.^{28,29} FIG. 2B: Bismark is an open-source tool than can be used for analyzing Methylation arrays and WGBS datasets used for statistical modeling. The package includes tools for processing, QC assessments, identification methylation loci of interest and plotting tools identify differentially methylated regions within the dataset to construct methylome maps.^{39,40}.

FIG. 3 provides an exemplary HCC Epigenotype Heatmap and Random Forest Machine Learning Output. FIG. 3 shows exemplary HCC Specific Epigenotypes. Differentially methylated blocks (200 bp in size) were identified using a Minfi Bumphunter. A random forest machine learning approach was then used to distinguish between Stage I/II HCC and control (high-risk and healthy patient) sample groups.³⁰ After model tuning with 200 bp binned blocks, the 100 most informative CpG sites (CpG sites that improved model performance) were identified. These sites provided nearly 100% accuracy when differentiating between HCC samples versus control samples as shown in the top clustering regions in light grey (control) and dark grey (HCC group), also highlighted by the black rectangular box to indicate clustering regions.

In reference to FIGS. 4A-4C, Liver Specific Methylated Markers. Differential methylation regions (DMRs) reported in average R were used to calculate mean methylated markers unique to the liver. Liver specific markers were selected based upon the following criteria: mean methylation in all liver specific tissue had at least a 40% mean R difference as compared to peripheral blood monocular cells (PBMCs), at least a 20% mean R difference as compared to 42 tissue types (referred to as “Other Tissue”.) Liver specific markers with less than or equal to 2 overlapping sites are considered liver specific methylation biomarkers to be measured in circulation. FIGS. 4A-4C: shows exemplary box plot between mean methylation status (mean β) of liver tissue, blood (or PBMC) and other tissue. A: miR443682 is reported. B: BDH1 is reported. C: miR583 is reported.

In reference to FIG. 5 provides an exemplary visual of liver specific methylation signatures. Dataset was used to compare methylation status in healthy patients, patients with obesity (without NAFLD), patients with steatosis, and finally patients with NASH. Methylation status was considered conserved if there was less than a 5% change in beta values between variables. Genes noted are associated genes. Note: y-axis reflects methylation status (beta value), x-axis reflects healthy or disease state; NAFLD denotes simple steatosis patients only. A: shows conserved region associated with GSTM1 gene. B: shows conserved region associated with BDH1 gene. C: shows conserved region associated with HLA-E. Liver specific methylation signatures (that are conserved on the liver) during varying disease states can be used as an indirect measurement of disease state from other material; for example, excess liver specific methylation signatures found in material such as but limited to in blood, saliva, urine or stool. Excess liver specific methylation signatures can indicate disease progression. For example, the concentration of liver specific methylation signatures in blood are stable during healthy state, defined as a healthy status; while the concentration of liver specific methylation signatures exceeds healthy status concentration and can be used an indirect measurement of excess liver scarring (e.g., associated with varying stages of fibrosis).

Table 1 (below) provides exemplary HCC Specific Differentially Methylated Regions (DMR) that exhibited differential methylation compared to control samples (e.g., comparing liver cancer high risk patents to healthy patients' liver tissue and liver disease liver tissue). Beta values are reported from 0.00 to 1.0. In one aspect, values greater than 0.5 are considered methylated, while values less than 0.5 are considered unmethylated. In this example, the table shows the most conserved regions based on averages with the lowest standard deviation. The average beta values for both HCC and control (normal and high-risk patients without HCC) are reported. Probe regions are reported in 200 base pair (bp) sequences; however, 1 kb and 3 kb regions can be assessed computationally using reported human genome coordinates.

Associated gene (or nearest gene), methylation direction (hypermethylated represented methylated regions, hypomethylated represents unmethylation regions) and distance to transcription start sight (TSS) are provided.

TABLE 1

| | | | | | Distance | | SEQ |
|---|---|---|---|---|---|---|---|
| Associated | Coordinate | | | | to | | ID |
| Gene | (hg19) | Direction | HCC | Control | TSS | 200 bp | NO: |
| POM121L12 | chr7:53104200- | hypo | 0.52580 | 0.92859 | 974 | ACCAGGAATTTTGGAGA | 1 |
| | 53104400 | | | | | AGTTCCCAGCAGCTCAGG | |
| | | | | | | AGAGTGGCTGGTGCCTTG | |
| | | | | | | TCCCAGGGGAGAGGGAA | |
| | | | | | | ATAAGCACAGGCCTATTT | |
| | | | | | | CTTTCTCAAGACGGAAAA | |
| | | | | | | ACCGAGCCCCAGAAAAT | |
| | | | | | | GGCGATAAGATGGGGAT | |
| | | | | | | GCAGGAAAAAGCTAGCT | |
| | | | | | | AATGAACAGTTCAGCAG | |
| | | | | | | TGGGCACCGTGTGGGAA | |
| | | | | | | CCTCTCCTA | |
| CSMD3 | chr8:114389000- | hypo | 0.53381 | 0.90536 | 281 | CACAAAGTTTCAGGGGT | 2 |
| | 114389200 | | | | | GAGTGTGACCCAGGGTT | |
| | | | | | | AGCTTCTAGGTCTGCTCA | |
| | | | | | | GGCCCTACCCCACACTAA | |
| | | | | | | ACTCAAATATCACTTCCC | |
| | | | | | | CTCAGGAGCTCAGGCGA | |
| | | | | | | TGGGAGAGGAAGATGAA | |
| | | | | | | GATGGAGTAGGTACCAC | |
| | | | | | | AGGTAATGAAAGAAACA | |
| | | | | | | GCACAGCGAACCGCAGA | |
| | | | | | | TATGCGGGAGGAGAACG | |
| | | | | | | GCAGAGCTAG | |
| AJAP1 | chr1:4842800- | hypo | 0.67204 | 0.89795 | 128231 | GGGTGAAGGCAGGAGAC | 3 |
| | 4843000 | | | | | TTACTTCGGAGTAGGACC | |
| | | | | | | CTGCCAAAAAGGCTGTG | |
| | | | | | | ATCCCTGTCCAGGGTGTT | |
| | | | | | | GCAATTCCAGCGGTGCTG | |
| | | | | | | GCGGAACTGGTGCTGGC | |
| | | | | | | ATTCTGCTGTCCACTCGG | |
| | | | | | | CCACGCCCTGGCTAATGG | |
| | | | | | | CACGCATCACATCTGGAT | |
| | | | | | | GTCGGTGACACAGCTGCC | |
| | | | | | | GCTGGCTGCTCACCAGGC | |
| | | | | | | CTGGC | |
| COTL1 | chr16:84600200- | hypo | 0.77334 | 0.93452 | 51384 | ctctgcctttttctgagtctgtttcctctcac | 4 |
| | 84600400 | | | | | catctctctctccatccgtccctccctgtat | |
| | | | | | | ctgcccctctctccatctcgctgctgtgtc | |
| | | | | | | cagctctctctctcgctcATTCCTTC | |
| | | | | | | CTCGCCATCTGGGGATCT | |
| | | | | | | CCCCGGCTCCGTGTCCCC | |
| | | | | | | TGCATTGCCCCGCGCCCC | |
| | | | | | | AATCACCGGCCAAAGCG | |
| | | | | | | CCCGCGGAGC | |
| TRIL | chr7:28862400- | hypo | 0.81368 | 0.94440 | 135446 | TCTCTCCCAACCCGCCCC | 5 |
| | 28862600 | | | | | CCCACCACACACAGGCA | |
| | | | | | | GGCTGTCAGTCACCCTGA | |
| | | | | | | AACAATAAGGCTTTTCAA | |
| | | | | | | AAGAGGAAAATCAAGCT | |
| | | | | | | TAAACGCTGGAGAGGAA | |
| | | | | | | CAGACTAAAAACTTAGG | |
| | | | | | | GGTCAAAGGCTCATAGA | |
| | | | | | | CTGTTGATTCATGTGTTC | |
| | | | | | | AGGCTAGAAAGGGCTGT | |
| | | | | | | GGATGTGCATGCCACCTC | |
| | | | | | | CAGGTTCT | |
| SLC25A36 | chr3:140696400- | hypo | 0.87627 | 0.97207 | 35822 | TGTCCAATCAGTCTGACT | 6 |
| | 140696600 | | | | | AATCTCCCTGAAATGCCA | |
| | | | | | | CCTTGGGCATTTCTTCAG | |
| | | | | | | AGCCCTCTCAGGCCCAGG | |
| | | | | | | AACTGGACTGGGCTGGTC | |
| | | | | | | CGCCTTGCAATGCCCTCT | |
| | | | | | | GTGGCCTGGCCCCACCCT | |
| | | | | | | CCATGCACACGCCCTTTG | |
| | | | | | | CACCTCATTGACTGGCAT | |
| | | | | | | CCTGCCATTTCTCCTGCA | |
| | | | | | | GATGCTATACCTCTTACC | |
| | | | | | | TC | |
| TMEM51- | chr1:15392200- | hypo | 0.86087 | 0.92717 | 86660 | GCGGACATGCATCACTA | 7 |
| AS1 | 15392400 | | | | | GCCCATAATTTAGGCAAT | |
| | | | | | | TTTGTTTAAAGACCACTG | |
| | | | | | | GTATTTCCTGGGGGAATT | |
| | | | | | | AATCGCCGGCTCCAAGA | |
| | | | | | | GTGCTTTGGGTCATTAAT | |
| | | | | | | CTTATATTCTTATTAAAT | |
| | | | | | | TCTCAGGAAATGACTATC | |
| | | | | | | TTATGAGGAGGGAGCAC | |
| | | | | | | TGCAGAGATTAGAGAAA | |
| | | | | | | TGCACACTGGGAGAGCA | |
| | | | | | | TGGGTAC | |
| TACC2 | chr10:123923200- | hyper | 0.22490 | 0.05341 | 155 | GGCGTCACCGCCGCCTTC | 8 |
| | 123923400 | | | | | CCCCTGAGTCCCTCCTTC | |
| | | | | | | CTGCGTCTGGTCATTCAT | |
| | | | | | | TCATTCACTGGCTCAGTC | |
| | | | | | | CGGTCCTTCTTCCTTCAC | |
| | | | | | | TTCTCATTCACTCAGACG | |
| | | | | | | GCTGCCTTATTTTCTCGG | |
| | | | | | | Cagttaggtgaccttggaccagtcaacc | |
| | | | | | | aacctcacctagcctcagtttgttagaaaa | |
| | | | | | | acaaggggggaggtgg | |
| NUDT16L1 | chr16:4738400- | hyper | 0.74087 | 0.26807 | -5194 | GGAGATGCCACTGTCCGC | 9 |
| | 4738600 | | | | | TTGGTTTAATGATCAATG | |
| | | | | | | AGCTCCCTGCCAGGAAA | |
| | | | | | | CCCTTTCTGACCTGGTTT | |
| | | | | | | GCCCCTCAGTCCCTCGGG | |
| | | | | | | CTCATACCTAGTGCCTGC | |
| | | | | | | GGCAGGACAGCCATGGC | |
| | | | | | | CGCCAACTCCACCAGCG | |
| | | | | | | ACCTCCACACTCCCGGGA | |
| | | | | | | CGCAGCTGAGCGTGGCT | |
| | | | | | | GACATCATCGTCATCACT | |
| | | | | | | GTGTAT | |
| NLRP2 | chr19:55494600- | hypo | 0.63801 | 0.94646 | 16958 | GTGTTTTTAAACAGAAAC | 10 |
| | 55494800 | | | | | ATAAAGGGAATGGAAGC | |
| | | | | | | CAGGGTGGCTAATGGTA | |
| | | | | | | CATAATGGCCTTAATTAC | |
| | | | | | | ACCACTGAAGGATCAAT | |
| | | | | | | ATTAGCGTAGCTCAGTCA | |
| | | | | | | CCAGCAGCCAAGGCACA | |
| | | | | | | GAAACTGGTACAGCAGG | |
| | | | | | | AAGTGGGAAACTGGGAA | |
| | | | | | | TCGGTCATCCCCGCCTGA | |
| | | | | | | TGCTCAGGGTGCACATTC | |
| | | | | | | CAAAAAGA | |
| MIR7160 | chr8:2041800- | hypo | 0.73593 | 0.95513 | 17231 | TTTGATCTCTAGATATTC | 11 |
| | 2042000 | | | | | TACTTTGGTCAATAAAGC | |
| | | | | | | AGAAGTCAGAGTGATTTC | |
| | | | | | | CTCATTCTGTGGTCTGTT | |
| | | | | | | CTTCTATTGCATAAAATA | |
| | | | | | | CCGAAGTGCCACCTCCTG | |
| | | | | | | GCTACCACAGGTGCTATC | |
| | | | | | | AGCCAGGCGGGCAGGCA | |
| | | | | | | GCATGAACAAGGCAACC | |
| | | | | | | ACTGGGGGATGGCGCAG | |
| | | | | | | TGCCACACGGCCTGTGGT | |
| | | | | | | AGGGA | |
| MYL1 | chr2:211179600- | hypo | 0.76860 | 0.95054 | 198 | ATCTATGTCTATTTCACC | 12 |
| | 211179800 | | | | | CAAGAACTCACCTTTGGA | |
| | | | | | | TTTGCACAACTATATCGA | |
| | | | | | | TTGACCTCTTAATTGAGT | |
| | | | | | | GTTTTTGAAATATTTAAC | |
| | | | | | | CAGGAGGACTATAACCA | |
| | | | | | | GTGTATCTAATAGTTTGG | |
| | | | | | | TGATCATATCTTGAGTAA | |
| | | | | | | CCAGTTGATTCTCTGCCA | |
| | | | | | | TGCCTGAAGACTTGGAAT | |
| | | | | | | TCTGCTTTAAAACACAGT | |
| | | | | | | TAC | |
| LOC391322 | chr22:24373200- | hypo | 0.30360 | 0.45705 | 199 | AACTGGGTTCCCTGACAC | 13 |
| | 24373400 | | | | | CCAGAGCTGCTCTGAAAT | |
| | | | | | | GGCCAGCGGCTTCACGC | |
| | | | | | | ATAACAAAGACCTTGCCC | |
| | | | | | | CTTGGCATTGTTTATTCC | |
| | | | | | | TTGACCTGTGCAGAGAGT | |
| | | | | | | CCTTCAAAAGAGGCTTTA | |
| | | | | | | CTGAAACTATTTCAGGGT | |
| | | | | | | TGCTTTCCAGGGGCAGAA | |
| | | | | | | TTTTCTAAAGAGAAAAAT | |
| | | | | | | TATTTATGCATATAGAAA | |
| | | | | | | GTT | |
| EGR3 | chr8:22570800- | hypo | 0.82287 | 0.96304 | −19907 | ATTTGTTTTCCACCTCCCT | 14 |
| | 22571000 | | | | | ATTCAGCAACCCATCTGG | |
| | | | | | | TCCTTATGCAAAGCACTT | |
| | | | | | | GCCTTCTCCCGGGATGGG | |
| | | | | | | GACACACATCAGAGCAC | |
| | | | | | | AGCGTGGTGTTTTCCCAA | |
| | | | | | | GCCCCCGAGGCACCTGTT | |
| | | | | | | CCCTCACATTACACCTTG | |
| | | | | | | AGGAGAGTCCAGTGACC | |
| | | | | | | TCTGCTGCAGAGGCGAC | |
| | | | | | | AAAGGCTCCCTGGGAAG | |
| | | | | | | CCGGC | |
| PCDHGA12 | chr5:140811600- | hyper | 0.33032 | 0.07478 | 1626 | GGCAGAGAACGCGCGAA | 15 |
| | 140811800 | | | | | AGGGCAGGGCCTTGGCC | |
| | | | | | | GGGAAGTACCGCCCAGC | |
| | | | | | | GAAAGGCTGGCAAGGGT | |
| | | | | | | GCGCAGCTGGAGCGTGG | |
| | | | | | | CCTCGGGTACCCCTTTCC | |
| | | | | | | AGGCAGCCGAACTCCCC | |
| | | | | | | GTATCCCAGCTCTGCTTG | |
| | | | | | | GCTGACTCCCATTTGTCT | |
| | | | | | | TGAGGGAGGATCCCCTA | |
| | | | | | | GTAGGACTGAATCAGAA | |
| | | | | | | GTGCGCCCGT | |
| GAS1 | chr9:89560800- | hyper | 0.42415 | 0.09046 | 1521 | CCTTTGGGAGGCGTTTGA | 16 |
| | 89561000 | | | | | CAGCTCAGAAAGCATGG | |
| | | | | | | CCTTTACTCAGGCTCAGA | |
| | | | | | | GACACACAAAAACCTTTC | |
| | | | | | | ACGTTTTCCTTTCAGCTT | |
| | | | | | | CGCAGCCCCGTCCTTCTC | |
| | | | | | | CACCTGCACGGGCACCC | |
| | | | | | | ACTGAAGCACAGGACCC | |
| | | | | | | TTCTGCCAAGCCATGAGC | |
| | | | | | | CTTCTCAGGCCCCTGCCG | |
| | | | | | | CCCGCCTCGCCTGAGGCT | |
| | | | | | | GCGCT | |
| CCDC177 | chr14:70038600- | hyper | 0.43002 | 0.06427 | 2888 | ATCCCAACCTCCAGGAG | 17 |
| | 70038800 | | | | | AAAGCATCATTTCACCTT | |
| | | | | | | TTGTTCTCCAGGCTTCAC | |
| | | | | | | TGTGGCCTCTCCATCCAT | |
| | | | | | | CTCAGTCCAGAAACAAG | |
| | | | | | | ATCCAGGTGGACTTTTCT | |
| | | | | | | TCTCCTTATGAGATGCAG | |
| | | | | | | GTGTTTGGACGACTTCTA | |
| | | | | | | GTTGCTCTCCTTTAAACC | |
| | | | | | | TGGAGACTGAGAagtcaaggt | |
| | | | | | | ggtattttggaaagcagct | |
| SIX2 | chr2:45233400- | hyper | 0.45956 | 0.07864 | 3091 | AGTTAGATTAACCTTCTC | 18 |
| | 45233600 | | | | | AACTGACTTTACTCTCAG | |
| | | | | | | GTTTTAAAGGCATCCACA | |
| | | | | | | TGATATGGTCTAGTTAAA | |
| | | | | | | AGGAGGAAGAGAGCAGC | |
| | | | | | | AAACTGGAAAAAAAGAA | |
| | | | | | | TAAAATATTTTCGTAGGT | |
| | | | | | | TCTGTACAGATCTGGTAG | |
| | | | | | | TATTATCTGCGTGTCTGG | |
| | | | | | | TTCACTGTGGTAGGCGCC | |
| | | | | | | ACAGAAATGGGTAGATT | |
| | | | | | | GATAA | |
| BASP1P1 | chr13:23422600- | hypo | 0.52606 | 0.89342 | 49620 | CACCTGCCAAACAGTGTC | 19 |
| | 23422800 | | | | | AAGGGTGAAAGGCAAAG | |
| | | | | | | ATCGAAGGCCAAAGTGC | |
| | | | | | | CCCTAGCGTCCCCTGCAG | |
| | | | | | | CAGGTCTCAGTCAACCTT | |
| | | | | | | AATAGATCTATTAATAGA | |
| | | | | | | AAGAGGCTGGACTGCGC | |
| | | | | | | AAGGGCAGGGGGACTAG | |
| | | | | | | CCTGGCTAAAAGCAAAA | |
| | | | | | | TAAAACACATCAACAGA | |
| | | | | | | CACACTCTTCTTTTGTTA | |
| | | | | | | GGAGGAGG | |
| ANKRD30A | chr10:37106800- | hypo | 0.62521 | 0.93957 | −307885 | AGCATGCACTTGAAGAT | 20 |
| | 37107000 | | | | | GGAGTTCAGATATCCAG | |
| | | | | | | GACGCTCCAGGCTTTGAC | |
| | | | | | | CCGCGATGGTGAGCAAG | |
| | | | | | | GGTTCCTCTTCCTTTTCCT | |
| | | | | | | ATGCTGGGCTTCTCAGTC | |
| | | | | | | AGTTTCATCACAAAAGGT | |
| | | | | | | GAAAGGTAGCTAGTAGT | |
| | | | | | | GACACATTCCAGCACCA | |
| | | | | | | GAAGAATTTCTAGAAATT | |
| | | | | | | ATTGTCTAAGGGTCTTTA | |
| | | | | | | ATTTCC | |
| TMEM132D | chr12:130434200- | hypo | 0.63365 | 0.94442 | −45730 | GGAAAAAGGGCAGGAGG | 21 |
| | 130434400 | | | | | GGTCTATGAAGCAGTCCT | |
| | | | | | | GGGGAGGACAGATTCGC | |
| | | | | | | AGTGTTTGAATGTGGGAA | |
| | | | | | | GAACTAATCAGTGAAAC | |
| | | | | | | ATTTTTTAAAGCCAAGTT | |
| | | | | | | TATCTTCGATCTCCTGCA | |
| | | | | | | GTTTCTGAAAGGATAGTT | |
| | | | | | | CAAAATTCCATCAAGTAG | |
| | | | | | | TTCTGACTTCCTGCTACA | |
| | | | | | | GAAATGCAATCGCATTG | |
| | | | | | | ACAATA | |
| LINC02500 | chr4:182287000- | hypo | 0.70955 | 0.97648 | −100918 | GAATCTTTTCCAGGAaaaaa | 22 |
| | 182287200 | | | | | aagaaagaaagaaagaaagTAAAT | |
| | | | | | | CCCACCCCCAAGCGTGCA | |
| | | | | | | GTCTGCCCACAGGAAGT | |
| | | | | | | GATTCATGGTGCTCCGAT | |
| | | | | | | TCGGGCAGACCTGACTGC | |
| | | | | | | AGGAGGCGGGAGGCTGG | |
| | | | | | | GAACCAGGTCTGGGAAA | |
| | | | | | | AGAGGGCAGAGAGAGGA | |
| | | | | | | TACCCAGACCTAATGGCT | |
| | | | | | | TCTGCTCTGTGGAAGA | |
| SMOC2 | chr6:168782400- | hypo | 0.72011 | 0.95703 | −59364 | GACTAATAATTTGAAACT | 23 |
| | 168782600 | | | | | ACAAGCTAACCAGATAT | |
| | | | | | | AGCTTTGCAGAAACCAA | |
| | | | | | | AGCCAACAACAACAAGA | |
| | | | | | | AGTATGAGCTGTTGACGT | |
| | | | | | | CAGCCCTGAAAGTGTTGA | |
| | | | | | | AAATAACAGTAGGCAGA | |
| | | | | | | GAAACACCTGCTCATGTG | |
| | | | | | | ATTGCCTCACAAGGATCT | |
| | | | | | | TGTTGCCTGCAATTTAGT | |
| | | | | | | CAGTGCCAAATGGGTGA | |
| | | | | | | TTATTCT | |
| MIR4689 | chr1:5870200- | hypo | 0.72993 | 0.96354 | 52501 | gcgggcattgtgaggattcagagagaat | 24 |
| | 5870400 | | | | | ccagagtttgtcctcagcacagggcccg | |
| | | | | | | gcGGGCTGGTGTGGGGGC | |
| | | | | | | CAGTGGCCGTCCTGGGTA | |
| | | | | | | AGCTCTGTGATCATTGCA | |
| | | | | | | GGTCCTCCCTGGCAGGTG | |
| | | | | | | AGACCCGCTCTGCTGGGC | |
| | | | | | | AGTGTGGCTGACAAGAG | |
| | | | | | | GAGGCTCTCGGGGAGCG | |
| | | | | | | CTCTTTGTCCCTATGCTG | |
| | | | | | | TG | |
| THEG5 | chr19:32147400- | hypo | 0.74652 | 0.97751 | 64313 | GAATCCCGCGCGGTGGA | 25 |
| | 32147600 | | | | | TGCAAGGTGGGATTCCA | |
| | | | | | | GCCCAGCGACGCGCCGC | |
| | | | | | | GTGGCCGGGAGTGCAGA | |
| | | | | | | CAGGTCACCGGTCCCCGG | |
| | | | | | | GTGTCTGGACCGCGCCGC | |
| | | | | | | TTCCTACTGGGCTTACTT | |
| | | | | | | CGGGATCTAGACTTCGGC | |
| | | | | | | CTCCGTCCTTGCAAGGGC | |
| | | | | | | CCAGATTTGATTCCGGTT | |
| | | | | | | CGTCGAGGCGGTCTGTAG | |
| | | | | | | CAGATA | |
| MROH5 | chr8:142632400- | hypo | 0.70518 | 0.92246 | −115170 | CTGATACAAAAGCTCCAC | 26 |
| | 142632600 | | | | | TTGCCGGATCCGGGCGA | |
| | | | | | | GCATTGCTGCTTCCACCG | |
| | | | | | | GGATGCGGGAAAAACAG | |
| | | | | | | TGCAGAGAGATCCTGAG | |
| | | | | | | CTTCCGCACGTCATCATT | |
| | | | | | | TCTCAGAGTCGCCATCTG | |
| | | | | | | TTCCTTCAAGCAAGTAAC | |
| | | | | | | ACCTTTGGGGCTGCAGTG | |
| | | | | | | TGGCTCAGAGCAGGTGTC | |
| | | | | | | TGGCCTCGTGTGAGATAC | |
| | | | | | | CCCAG | |
| MRPL36 | chr5:1786800- | hypo | 0.76516 | 0.97224 | 13051 | TCCCGTTCCCGGTGTTGG | 27 |
| | 1787000 | | | | | GGCCAAAGCTCTGCTTGA | |
| | | | | | | TGGGGTCTCAAGTTGCCT | |
| | | | | | | TTCTTCCCCCTCCCCCCG | |
| | | | | | | CCCGCAGCAGTCCGGAT | |
| | | | | | | GCCGCCCGCATCCGAGTC | |
| | | | | | | AAGCGCTACCGCCAGAG | |
| | | | | | | CATGAACAACTTCCAGG | |
| | | | | | | GCCTCCGGAGCTTTGGCT | |
| | | | | | | GCCGCTTCGGGACGTGCA | |
| | | | | | | CGGTGCAGAAGCTGGCA | |
| | | | | | | CACCAG | |
| LINC00602 | chr6:166270200- | hypo | 0.66691 | 0.87160 | −130739 | TACTCCCATGCAGATTCA | 28 |
| | 166270400 | | | | | GCATCCCTTTTCTATAAC | |
| | | | | | | AGAGCAAACCGAGCTTT | |
| | | | | | | ACTGGGTTAGCTCCACAT | |
| | | | | | | TTGACTGCAGACACTGTC | |
| | | | | | | CCTTAAGCTAGATTTTTA | |
| | | | | | | CCAACCACAATAATGGC | |
| | | | | | | ATCTATTTCTCTTGCATC | |
| | | | | | | ATTTCCATTCAACAAATA | |
| | | | | | | TACATATTTTCAATGGCC | |
| | | | | | | TAAAACTCCAAGTATGCA | |
| | | | | | | AAGC | |
| MEGF6 | chr1:3532800- | hypo | 0.72763 | 0.91988 | −4828 | CCATGCCTCTTTGCTGTT | 29 |
| | 3533000 | | | | | TGTGGAAAATAAAGCAT | |
| | | | | | | TCTATAGGCGGAGCTAGT | |
| | | | | | | GAACGCCTCTTTTAAAAC | |
| | | | | | | ACGAGTCTCCACACTTCC | |
| | | | | | | CTGTTCACTTTGGTTCCA | |
| | | | | | | GCATCCTGTCCAGCAAAG | |
| | | | | | | AAGCAATCAGCCAAAAT | |
| | | | | | | GATACCTGGAGGCTTATC | |
| | | | | | | TGAGGCCAAACCCGCCA | |
| | | | | | | CTCCAGAAATCCAGGAG | |
| | | | | | | ATTGTT | |
| MIR4456 | chr5:554800- | hypo | 0.78172 | 0.95218 | −18903 | CGCCAAAGTATTTTGAGT | 30 |
| | 555000 | | | | | CCCTGAGGTCAGGCCAG | |
| | | | | | | AGCAGCCTCGTGCCCCCA | |
| | | | | | | CTACCTGAGAAGTGCCCC | |
| | | | | | | CGGCCTCCCAGGGGTGCT | |
| | | | | | | GTGCTGGGCAACCACTCA | |
| | | | | | | TCCCTGGAGCCCCTCGTA | |
| | | | | | | TGCCCTCGCTGCCCTCCG | |
| | | | | | | AGTGGGCTAGCCACCCCT | |
| | | | | | | GCAGCTGCCGGGGAGCC | |
| | | | | | | AGCTGACCTCCGGGGCA | |
| | | | | | | GCCTC | |
| MIR6072 | chr10:2125200- | hypo | 0.83645 | 0.98283 | −7017 | CAACCAGACTGGCCCCTT | 31 |
| | 2125400 | | | | | TACTCCTTCCTCCCTCAA | |
| | | | | | | CCTGTCCATATGCTATTA | |
| | | | | | | TGTTTGCCTACACATACA | |
| | | | | | | GGTTCACTGTTGGAAATC | |
| | | | | | | TGTGAGTGGGACAtttttctga | |
| | | | | | | atttttattttagattcagggggtacatatgc | |
| | | | | | | aggcttgttacctgggtatattgtgtgatgc | |
| | | | | | | tgaggcttggggtacaaatgatccc | |
| LINC02667 | chr10:130844000- | hypo | 0.84962 | 0.97367 | 133003 | CACGCATCTCTCTGGCCT | 32 |
| | 130844200 | | | | | GTTGGCTCACATTTGGGG | |
| | | | | | | GTGTGGCATTTGTGCGTG | |
| | | | | | | AAGTGGAGCCTGGTCCCT | |
| | | | | | | GGTGACCCAGGTACAGTT | |
| | | | | | | TACGTCTGGAGCCAGCAT | |
| | | | | | | TTGGAAAGTTGCCTCCAG | |
| | | | | | | GGCCCTGGATTTGGAGG | |
| | | | | | | GAGCCAACTGCCAGAGT | |
| | | | | | | CTCAAGCAGGTGAGGGT | |
| | | | | | | GGGGTGCAGGAGAGGAG | |
| | | | | | | CGTTCA | |
| MIR4472-1 | chr8:143203400- | hypo | 0.78733 | 0.88999 | −54200 | AGATATCCAACTTTAAAT | 33 |
| | 143203600 | | | | | AAGGGACAAGAAAATCG | |
| | | | | | | ATACCTATGAGATGCTCA | |
| | | | | | | CTGTTTTCAAGTAATAGT | |
| | | | | | | AATCAATCTGTTTTTGAA | |
| | | | | | | TCCTACCAGGCTGTCTAC | |
| | | | | | | ACTGGGTGCAtggattagttagtt | |
| | | | | | | attgatgcataaaaaccacaaaaaactta | |
| | | | | | | gtagctcatgattgacatgctcagccattt | |
| | | | | | | aggcttggct | |
| EFNA5 | chr5:106709200- | hypo | 0.87019 | 0.94734 | 297338 | TTACTCCAGCTGAGGCAT | 34 |
| | 106709400 | | | | | CAGATGTGTGGGTGAAG | |
| | | | | | | AAACCAccagacgcggtggctcac | |
| | | | | | | gcctctaataccagcactttgggaggccg | |
| | | | | | | agggggcggatcacgaggtcaggagt | |
| | | | | | | ttgagaccagcctggccaacatgctgaa | |
| | | | | | | accccgtctctactaaaagtacaaaaaag | |
| | | | | | | aaaaaaaaaatagccgggcgtggtggc | |
| | | | | | | g | |
| LONRF3 | chrX:118107400- | hyper | 0.34876 | 0.32258 | −1077 | gctcccagccgccgtgatatatcagcttat | 35 |
| | 118107600 | | | | | gtcatcctgacaCCTACGCAGA | |
| | | | | | | TGTCGGCTCGAATCCACT | |
| | | | | | | TTGCCTGAGCATTGTCTC | |
| | | | | | | AGAGAAATCTAATTTAA | |
| | | | | | | AAATTAGGCAGCAAATA | |
| | | | | | | GAAAATATATTTGACTGC | |
| | | | | | | TAGAGATGCAATGGGAC | |
| | | | | | | TGGGAGCCCAACAAAGG | |
| | | | | | | ATCTTAGGCAAAAGAAA | |
| | | | | | | TCCAAGTTG | |
| TBC1D28 | chr17:18553600- | hyper | 0.57564 | 0.48250 | −5960 | CTCGACATGATTGTCCCC | 36 |
| | 18553800 | | | | | TGTCGGCCCTGCGCCGCC | |
| | | | | | | GAGGCCGAGTGACAAGG | |
| | | | | | | CGCAGGAACCGCCTATTC | |
| | | | | | | GGTGGGGCTACCAGCGG | |
| | | | | | | AAGGAAGTTTACTCTGCA | |
| | | | | | | GTGGACACTGTTTTAGCA | |
| | | | | | | CTTTAAGAGTAGCAGGA | |
| | | | | | | CCATTCCTAAATAATTGA | |
| | | | | | | GGTTCCCCCTCCTCCACA | |
| | | | | | | CAAAATTTTAAAATTTCA | |
| | | | | | | CGGAT | |
| NLGN4Y | chrY:16632400- | hyper | 0.62325 | 0.52761 | −1996 | GGTTAACCCAGATTGCCT | 37 |
| | 16632600 | | | | | TCGGTGCCTGAGGGACTT | |
| | | | | | | GATATTTTCTCATTATGA | |
| | | | | | | AGACTTTCCCATCTAGTG | |
| | | | | | | AACGCTGCAAATAAAAC | |
| | | | | | | TGCTCAGAATCGGTCTGA | |
| | | | | | | ACTGCCTGTTTCCGTGGT | |
| | | | | | | AGCAGGCCTCTGTCTGGA | |
| | | | | | | GGATAAATCTGCACCATC | |
| | | | | | | ttcttttttttttttttttttttGGGTTTCA | |
| | | | | | | GCCAGTTG | |
| USP3 | chr15:63758800- | hyper | 0.65927 | 0.54275 | −37810 | TGGGCTAAAAAGGATAG | 38 |
| | 63759000 | | | | | ACAAGGAACACATCCTG | |
| | | | | | | GGCCGGTAATTACGCAA | |
| | | | | | | AGCATTATCTCCTCTTAC | |
| | | | | | | CTCCTTGCAGATTTTTTTT | |
| | | | | | | TCTCTTTCAGTACGTGTC | |
| | | | | | | CTAAGATTTCTGTGCCAC | |
| | | | | | | CCTTGGAGTTCACTCACC | |
| | | | | | | TAAACCTGAAACTAATA | |
| | | | | | | AAGCTTGGTTCTTTTCTC | |
| | | | | | | CGACACGCAAAGGAAGC | |
| | | | | | | GCTAAG | |
| UTP14A | chrX:129065600- | hyper | 0.49800 | 0.32700 | 25560 | TGTTGAGTGCACCCTCTT | 39 |
| | 129065800 | | | | | GTTATAAGAGCTGATATC | |
| | | | | | | CCGAAGTATACATGTGCA | |
| | | | | | | AGGAACATGCCTGCTTCT | |
| | | | | | | TCAGTGTGCATTCCAACA | |
| | | | | | | TGCCACAGAAATGTGGTT | |
| | | | | | | TACATCATAGCTTCTACC | |
| | | | | | | GCCGAATAGTCTATCTTT | |
| | | | | | | AGATTTTGTTTACATGGT | |
| | | | | | | TTGTCTCAATTAGCTTAA | |
| | | | | | | TACATAATTAGCTATTGT | |
| | | | | | | TG | |
| FOXD1 | chr5:72731800- | hyper | 0.34844 | 0.10350 | 12702 | ggaggattccttgagcctaggagtttgag | 40 |
| | 72732000 | | | | | gctgcagtgagctatgattgcaccactgc | |
| | | | | | | actccagcttgggtaacagagcaagaac | |
| | | | | | | ctatcttttttttttctaaaaaaaaaaaTTG | |
| | | | | | | GAATATGGAAAGTTGAT | |
| | | | | | | CGGATAAACGTTGTTTTA | |
| | | | | | | TCTTTGTTAAAagataaatggta | |
| | | | | | | aatattaaatttcccaaagttggta | |
| SLC22A31 | chr16:89268000- | hyper | 0.49751 | 0.24828 | −1155 | GCAGGTATAGTCCCATGA | 41 |
| | 89268200 | | | | | CCACCCCCCGAGCATGG | |
| | | | | | | GGTTATCTCATTCTTTTTT | |
| | | | | | | TTGGGGGCGGGGGGgacgg | |
| | | | | | | agtctctctctgtcgcccaggctggagtg | |
| | | | | | | cagcagcgcgatctcggctcactgcaac | |
| | | | | | | ctccgcctcccaggttcaagcgactctcc | |
| | | | | | | tgcctcagcctcctgagtagctgggatta | |
| | | | | | | caggtaggcacc | |
| PRRX1 | chr1:170630000- | hyper | 0.36384 | 0.08436 | −3172 | TTGGCTCTCCCAAGTTAT | 42 |
| | 170630200 | | | | | TCTTGGTCCACACTTGGC | |
| | | | | | | CAGCACTAACAGAGCAC | |
| | | | | | | ACGTGGCAGTATTCACAA | |
| | | | | | | ATATTAGACATATTTTAA | |
| | | | | | | TTGGAGGTGGCAACAGG | |
| | | | | | | GGAGAAATATTTTTGGTT | |
| | | | | | | CCTTCTCACCTGTGCCTC | |
| | | | | | | CTTAACCTGCCCAGGGGA | |
| | | | | | | CTGTGAAAAGAAAAAGA | |
| | | | | | | TTCTAGAAGCACACTTTA | |
| | | | | | | GAAGA | |
| LINC01381 | chr2:25439200- | hyper | 0.39563 | 0.05478 | 12118 | gtctattatttttatgcctttgggtcctcatat | 43 |
| | 25439400 | | | | | gcatctgctattttcaagtgcctttagctcc | |
| | | | | | | aaataatccttatgccaaaatggcatatttg | |
| | | | | | | ggggcaacacattctgccacccttcaGT | |
| | | | | | | ACCTTTGGCCAAACAGA | |
| | | | | | | GGTTTGCTTTCCAGACCT | |
| | | | | | | CCTGGGTTCTGTGACTGG | |
| | | | | | | CAACTGCACAAGCAGGG | |
| | | | | | | CACGCTC | |
| ACTG1 | chr17:79481400- | hyper | 0.56536 | 0.21853 | −1675 | GTGGGTGGCCCGTGTACA | 44 |
| | 79481600 | | | | | GGAATGGGATCGCACGG | |
| | | | | | | TGGTCCAGATCGTCTGTG | |
| | | | | | | CACCCATCCTTGCCCTGC | |
| | | | | | | CTCCCTCCTTCTAGGGGG | |
| | | | | | | TTGGCTGGAACTCGGGCC | |
| | | | | | | CCGCTGGCCGGCGGTAG | |
| | | | | | | GAGCTGGAATGCCCGAG | |
| | | | | | | CTCTGAGAAGCTCTTCAT | |
| | | | | | | CTATCAGCAGTCAGCTTT | |
| | | | | | | CCACTTAAGCAGAGCCA | |
| | | | | | | AGTTTA | |
| HOXA11- | chr7:27231400- | hyper | 0.50886 | 0.08831 | 6473 | AGCTCGCCTGGGCTCTGT | 45 |
| AS | 27231600 | | | | | AGCTTCTCTCAGTAGAAA | |
| | | | | | | GGATTCTTCCATGGCTGG | |
| | | | | | | GGTGAGCGGGCTGGGAC | |
| | | | | | | ACCTGCAGGAAAGGCTC | |
| | | | | | | CTCAGGAAGAAATAACT | |
| | | | | | | GGACCTTCAATAACCTTT | |
| | | | | | | TTTTGCAGTCTCAATATT | |
| | | | | | | GGCCTTAGCTTGGAGTCA | |
| | | | | | | CTGGGCTCAGGCTTCAAT | |
| | | | | | | TCCCAGTCAGAGTTATTC | |
| | | | | | | ACTTG | |
| FOXC1 | chr6:1620600- | hyper | 0.54964 | 0.12087 | 10550 | GTGGATGAGAGGGTGGG | 46 |
| | 1620800 | | | | | GCCCTCTACGGAGATGA | |
| | | | | | | GATCTTACCTATGTAAAA | |
| | | | | | | CCACGCTCCAGAGTTCCT | |
| | | | | | | AAACCACAACCCTACCTG | |
| | | | | | | GGAAGCACCCAGGGCTG | |
| | | | | | | AGAGTTGGTCTTTAAAAC | |
| | | | | | | ATTCTTAGCCTATAACCT | |
| | | | | | | CTTCTTTAAAAATCTGTT | |
| | | | | | | GTCTCTTCTAGAACAAAT | |
| | | | | | | TATTTTAAACCAGTTCAA | |
| | | | | | | GAAAA | |
| DUSP10 | chr1:221737200- | hyper | 0.76214 | 0.30577 | 172942 | GGCTCTTCCCATCGACAA | 47 |
| | 221737400 | | | | | CTGGGCATAGCTGTTGGT | |
| | | | | | | TTTGTGCCAATCACCCCA | |
| | | | | | | CTTGGGAAACCCTGAGCC | |
| | | | | | | ACCATGCCTCAGGGAAA | |
| | | | | | | GTGGTGGCACTTCCTGGT | |
| | | | | | | GGCACCAGGAATCGCAC | |
| | | | | | | AAGGCCATCACTGCCGCC | |
| | | | | | | TGGTGCACAGGGCTCCCG | |
| | | | | | | CGGCTGGCATCTCCAGAG | |
| | | | | | | CCTGGAGGAAGAGGGTA | |
| | | | | | | GAGGA | |
| LOC101929268 | chr8:49292400- | hyper | 0.63222 | 0.15952 | −171627 | CTACACCTGAGAACGAC | 48 |
| | 49292600 | | | | | ATTCACTCTGCTTCATTG | |
| | | | | | | AAAACAATCTATCCCGTG | |
| | | | | | | TGTGGAAGAAGCATGCC | |
| | | | | | | TTGCTGGGGTCAGGGCCA | |
| | | | | | | GGGACAAAGCTCAGAGG | |
| | | | | | | CCATGCTGAGTTTTCATC | |
| | | | | | | AAACACCTGCTGAGCAG | |
| | | | | | | CTGGCACGTGCCAGGAC | |
| | | | | | | ACGGTCTAGGCTGAAGG | |
| | | | | | | CTGCCATCGTAGTGCAGA | |
| | | | | | | ATAGAATC | |
| ENGASE | chr17:77174000- | hypo | 0.49101 | 0.93834 | 103081 | ACTCATACCCCTCCTCCT | 49 |
| | 77174200 | | | | | CCTCCGCGTTATTAATCC | |
| | | | | | | AGCAGTTCCCTCCTCCTC | |
| | | | | | | TGGGTCTGATGCTGCAGC | |
| | | | | | | CCTGGAGTATTGCAGTAT | |
| | | | | | | GTGTGTCCACTCCTGCTG | |
| | | | | | | CCTGCCCAGGATTGGTTT | |
| | | | | | | GGGCGAAGTAAAACTGG | |
| | | | | | | GAGTTTTAAGTATAAACG | |
| | | | | | | GCAACAACAGCTCTTTGA | |
| | | | | | | GAGCATTTTAGAAAAGT | |
| | | | | | | GAAA | |
| DPP10 | chr2:116122000- | hypo | 0.54166 | 0.89872 | 20709 | CCGGGGCTTGGGGGGCG | 50 |
| | 116122200 | | | | | GCCGGGCAGGGGCCACT | |
| | | | | | | CGGTGCTGCGGGGGCTG | |
| | | | | | | CTGCCCTGCGGGGCATCG | |
| | | | | | | CTCTTTCTCGGCCGGGGC | |
| | | | | | | ATCCCCCGAcggggcgggcgg | |
| | | | | | | cggcggcggcgggggcTGGAAC | |
| | | | | | | ACCCGGGACCGCTTGCTG | |
| | | | | | | ACCAGCTTCGAGTTGACC | |
| | | | | | | TGGGGAACCCCTGCGGC | |
| | | | | | | CCTGGGGAGCCCGGGCC | |
| LSAMP- | chr3:115984200- | hypo | 0.60996 | 0.93443 | −94571 | TGACAGATTTTTATTATA | 51 |
| AS1 | 115984400 | | | | | ATAGTCATCTCCTTCTAA | |
| | | | | | | AATCTGGTGAGCAGCTTC | |
| | | | | | | CCTGCCGACTCAATAATG | |
| | | | | | | CCTATTGCTGAACATCTG | |
| | | | | | | CAGTTACACAAAGGACT | |
| | | | | | | ATTACTGTTGGCTGAAAG | |
| | | | | | | CTTTCTTATAGAGGATAC | |
| | | | | | | TTCTTTAGAAAAGGAAGT | |
| | | | | | | AGCAGTCTTTACCTGCAG | |
| | | | | | | TTGGGTGGGGATACACTA | |
| | | | | | | CAG | |
| DLGAP2- | chr8:1527800- | hypo | 0.55093 | 0.87382 | 41930 | TCTCTTCCTGCCCAGCGT | 52 |
| AS1 | 1528000 | | | | | CCACTTACTATGGTCTTT | |
| | | | | | | AGCCCATGGCTGGCATTC | |
| | | | | | | AGTAAGTACTGGGGGGT | |
| | | | | | | GACAGAGTAAGAGAGAA | |
| | | | | | | CAGGTGAGTGGGAATGC | |
| | | | | | | ATCAGTGAATATGTGTGA | |
| | | | | | | AAGAGAAAGTTCATGAA | |
| | | | | | | AGCAGAAGGAAATAGAA | |
| | | | | | | TGAAAGAGGAAGCGAAG | |
| | | | | | | GGGGTAAGCAAATGGAA | |
| | | | | | | AAGAGAGAC | |
| PXDN | chr2:1879800- | hypo | 0.60324 | 0.91557 | −131613 | ATACGTCAAGGACTCTGA | 53 |
| | 1880000 | | | | | AGCCGTGAGAGAGGGGG | |
| | | | | | | AAGAACAACAGTAGAGA | |
| | | | | | | GGATGCCCAGCTGGTAA | |
| | | | | | | GAATCGAGTGTTTATGAA | |
| | | | | | | GTTTTAGTCAATTGATGA | |
| | | | | | | ATCTCATTGGCTAAAATC | |
| | | | | | | AAGAAACGCTCCGCCTCT | |
| | | | | | | TTGCAAATATGTATGAAG | |
| | | | | | | GAGAGAAGTGCCTAAAC | |
| | | | | | | TTCTATGTCTGATAGCAT | |
| | | | | | | TTGACC | |
| LINC01103 | chr2:105118400- | hypo | 0.64523 | 0.94284 | 8467 | ACTTTTAATACTTAAATA | 54 |
| | 105118600 | | | | | ACGACAATAATAATAAT | |
| | | | | | | ATACAAGGTTAGTTCCAA | |
| | | | | | | GGCATGTAGAGGTGATG | |
| | | | | | | GCCAGCAGAGGTGAAGC | |
| | | | | | | CAATCCACCCTTTCTGGG | |
| | | | | | | CTAGGGGAAGCCCAGAT | |
| | | | | | | GGTCTTCCGCTCGCTCGG | |
| | | | | | | GGTGAGGCACTCCCCAG | |
| | | | | | | GGTCCCGGCCTGGCTGCC | |
| | | | | | | CATCCCCCACCAAGTCTC | |
| | | | | | | CCAGGCC | |
| WWOX | chr16:78496400- | hypo | 0.32745 | 0.61566 | 362949 | GGTTGACAAGTAAGGTT | 55 |
| | 78496600 | | | | | GCCAACGGAAGCCAGTT | |
| | | | | | | GCTCTCTTCTGATGCTGG | |
| | | | | | | AACTTACAACCTGTCATC | |
| | | | | | | TTTTGTTGTCTGCAGAGC | |
| | | | | | | AGAGAACATAGCCAAGC | |
| | | | | | | CGGCAGGGACAGTCTGTT | |
| | | | | | | GTATGAGTCTGGATCTCA | |
| | | | | | | TTGCAATGATGCAATTTA | |
| | | | | | | TTTACACATACCAAGGAT | |
| | | | | | | GAGCCATAAATCCTTACC | |
| | | | | | | ACTGC | |
| PBX1 | chr1:16454460 | hypo | 0.61338 | 0.89966 | 15741 | TTGGGGCCCAGGGACAC | 56 |
| | 0-164544800 | | | | | ATCTTCCCTGTCTTTCTCT | |
| | | | | | | ATCTCCCTCCTCCCTTCTT | |
| | | | | | | ACCCCAGGCTTGGGAAG | |
| | | | | | | TCTGAGTGTTTCTGGAAG | |
| | | | | | | TCTGCTGCTGTGTGGGAA | |
| | | | | | | TCACTCTTGTCTTGCTGC | |
| | | | | | | TTAAGACACGTGGGAGC | |
| | | | | | | TGTTAATGGCTCACATTT | |
| | | | | | | CAAAGCCTGGCTTTGGGG | |
| | | | | | | TAGGATTTAATGCAAGCC | |
| | | | | | | ACC | |
| LINC02645 | chr10:2542200- | hypo | 0.64677 | 0.92848 | 1354 | AAAACTCATGACTCTGAC | 57 |
| | 2542400 | | | | | TGGTTAAAATGAGTGTCA | |
| | | | | | | GTGTTCTCCATCTGTCCT | |
| | | | | | | CGTAACAGCATCACTGGC | |
| | | | | | | TCTATATTGTCAGATCTT | |
| | | | | | | TAATACTAACTTTCTGCC | |
| | | | | | | CAGTGAGCAATGACTCAT | |
| | | | | | | ACAAAGCTCAGTGCCCAT | |
| | | | | | | TGGTTCTTTTCTCAGAGT | |
| | | | | | | CTGTCCAATCCTAGGGTC | |
| | | | | | | ACAGAAGACTGCTTGGG | |
| | | | | | | TTC | |
| LINC00200 | chr10:1334200- | hypo | 0.60145 | 0.88190 | 128592 | AAATCAACTCCAGCTCCC | 58 |
| | 1334400 | | | | | TCTGCGAGGCTGGCATGA | |
| | | | | | | TTGTTCCATTTCACCCAG | |
| | | | | | | CCACTCAGTCCCTTGCCT | |
| | | | | | | GTTACACTGTGGGGCTGA | |
| | | | | | | AACCTAGGCAGGCCGAG | |
| | | | | | | CCCCAGCCACCCCAGCTC | |
| | | | | | | TGAGCCGCCTCCCCACCC | |
| | | | | | | CTCACCTGATGGTCCACT | |
| | | | | | | GTGCTCCCGTAGAGCCCG | |
| | | | | | | TTGAGGTTGGCGTAGTGG | |
| | | | | | | CAG | |
| VCX3A | chrX:6452600- | hypo | 0.71574 | 0.96785 | 459 | AAGAGGGGTGAGAGCCT | 59 |
| | 6452800 | | | | | CGAGATGATGGGTAGTCT | |
| | | | | | | CTGACTCTTAACTGGATC | |
| | | | | | | TGCTTCACACCTAGGTCC | |
| | | | | | | CGACATCTGTGGCCCTGG | |
| | | | | | | CACCAAGAAGGTTCATGT | |
| | | | | | | CATCTTCAACTACAAGGG | |
| | | | | | | CAAGAACGTGCTGATCA | |
| | | | | | | ACAAGGACATCCGTTGC | |
| | | | | | | AAGGTGTGCCTGGGGGT | |
| | | | | | | GGTGGCAAATGGCTGTC | |
| | | | | | | ATGGGGA | |
| ZFPM2 | chr8:106406400- | hypo | 0.66662 | 0.91699 | 74729 | CTGGAGTTTCTGATAATT | 60 |
| | 106406600 | | | | | TCCAAATTGAACAACCTC | |
| | | | | | | AGTCCTAATGAGTCAGA | |
| | | | | | | GGCTTGTGCAATATTTTC | |
| | | | | | | AAACCTCAGGAACATCTT | |
| | | | | | | TTTCATTAGTTGTGCAAT | |
| | | | | | | AAAGATGGTAGGCCTAT | |
| | | | | | | CTCTGTGATGAGCTGTTT | |
| | | | | | | TTTTTTCTCAAAGTTTGA | |
| | | | | | | TGAGATTCGCCGTAGAAT | |
| | | | | | | TCCTTCTCACATAGTCTT | |
| | | | | | | GGGC | |
| COL14A1 | chr8:121208400- | hypo | 0.44921 | 0.69090 | 71159 | GGACACAGCCCTCAATCT | 61 |
| | 121208600 | | | | | TTGCCCCTTGCAGATGAT | |
| | | | | | | GTGCCTTTTGAGAGGCAC | |
| | | | | | | CAGGTGACTGCTCACAGT | |
| | | | | | | ATGCAAGCGCATTATCTA | |
| | | | | | | AAAGCACTCCCTCATTTG | |
| | | | | | | GAAACACTTCAATGAAA | |
| | | | | | | AAGTGAAATTTTGTTCAC | |
| | | | | | | AATCCACTCCCATTAATT | |
| | | | | | | TCAGAATTTTCAAggctggg | |
| | | | | | | caccgtggctcacccctgt | |
| LINC01587 | chr4:5570000- | hypo | 0.76720 | 0.97083 | 43217 | CTGTATAGCATCTTTCTC | 62 |
| | 5570200 | | | | | CACCTACCAAATGGGGG | |
| | | | | | | GCCGTGCCATCTCTGCCT | |
| | | | | | | CTCGGTGAGGAGATATTA | |
| | | | | | | CGACACAGCCAGTGAGA | |
| | | | | | | TTCACAAAGCACCTTGCA | |
| | | | | | | GAGTGGGCCTACTCTACA | |
| | | | | | | CTCTGGTTTTCAGTTTTTA | |
| | | | | | | TTGGAGTCACAGGTGGG | |
| | | | | | | AATGCATCAAACAAAAG | |
| | | | | | | ATATAGGCTCTGGAATGT | |
| | | | | | | ACGGG | |
| MIR561 | chr2:189193600- | hypo | 0.56471 | 0.75563 | 31481 | TGCACACATCAGCAAGA | 63 |
| | 189193800 | | | | | GACAGGAAAGGTCATCT | |
| | | | | | | CAGAGCCGGGAGTCACA | |
| | | | | | | GCTTTCTCCTCCATCTGT | |
| | | | | | | CCACACCACCTCCCTCCT | |
| | | | | | | CCTTCATCCGTCCAAACC | |
| | | | | | | ATGTTTCTCCTGCTGCAT | |
| | | | | | | CCGTCCACACCACCTCCC | |
| | | | | | | TCCTCCTGCATCCGTCCA | |
| | | | | | | CACCACCTTCCTCCTGCT | |
| | | | | | | GCATCCGTCCACACCACG | |
| | | | | | | TCCCT | |
| AJAP1 | chr1:4794000- | hypo | 0.79476 | 0.97888 | 79431 | GCTGGCCAAGTTGAGCG | 64 |
| | 4794200 | | | | | AGGACATTGTCTTTTGTT | |
| | | | | | | GGGAAAGGATACTTTTTA | |
| | | | | | | AGTGTTGTTTCTAAATTT | |
| | | | | | | TACTACATTTGCTGAAAA | |
| | | | | | | GACAGGACAGGAAGCTT | |
| | | | | | | TTAAGAATGAACAATCTC | |
| | | | | | | CCAGCCTGTGTGGGTTGG | |
| | | | | | | TTTGAGCTGTGAGAGGTG | |
| | | | | | | CAGCTGGGCTTCCAAGTA | |
| | | | | | | GGCTTATATTTCCAGACT | |
| | | | | | | CCCA | |
| RIMS1 | chr6:72806800- | hypo | 0.78130 | 0.93919 | −115696 | GTGAAATTCGAGATTGCG | 65 |
| | 72807000 | | | | | TATTTCACCAACACAGGA | |
| | | | | | | GCACAGCCCTCGGGAAA | |
| | | | | | | CTCAGCCTAGTCAGGCAG | |
| | | | | | | TAGAGAGTTGTCCCGGA | |
| | | | | | | GAGAAGTGATCCTGCAG | |
| | | | | | | ACTCGAGAAGGGGCATG | |
| | | | | | | ATGATAGCACACGTCTGT | |
| | | | | | | TGAGCACCCAGTCTGTGT | |
| | | | | | | GCCGGGTGTGTTACCTCT | |
| | | | | | | GTGACCTCATTTGGTCAA | |
| | | | | | | ACGAGG | |
| OCA2 | chr15:28272600- | hypo | 0.78268 | 0.93763 | 71761 | CGCAGAGCTGGCCGGGG | 66 |
| | 28272800 | | | | | CATTCGTGCGTGTTCACG | |
| | | | | | | accgagaacttttcacagagagaggactt | |
| | | | | | | ctgtcctgatagacgtcggtgaggactga | |
| | | | | | | atccccacttacaggcgtgcacatcAG | |
| | | | | | | GGACTGAtggacatcgctgaggac | |
| | | | | | | cgatcccagcttacaggcgtgcacactg | |
| | | | | | | gaggctcggaagagctgactgtgactca | |
| PBX1 | chr1:164545400- | hypo | 0.66751 | 0.81093 | 16541 | CCCGAGGACTGGGATGA | 67 |
| | 164545600 | | | | | AGAGATGGACGGAGAGT | |
| | | | | | | GGGAACCCCCAGTGATTC | |
| | | | | | | AGAACCCTGAGTACAAG | |
| | | | | | | GTGAGTTTGGGGCTCTGA | |
| | | | | | | GCAGGGCTGGGGCTCAC | |
| | | | | | | AGTGGGGAGTGCACCAA | |
| | | | | | | CCTTACTCACCCTTCGGT | |
| | | | | | | TTCCTTCTCCCTTCTGCA | |
| | | | | | | GGGTGAGTGGAAGCCCC | |
| | | | | | | GGCAGATCGACAACCCA | |
| | | | | | | GATTACAAG | |
| MIR4251 | chr1:3037200- | hypo | 0.76729 | 0.90892 | −7239 | GTGGATAAGTTTTTCTCC | 68 |
| | 3037400 | | | | | ACGTGGGCCACATTCTTA | |
| | | | | | | CTAGAAAAGAAACCCCG | |
| | | | | | | GCCCTTTAGCTCATCACC | |
| | | | | | | CTCTGCTCTGGCTTTCCC | |
| | | | | | | CGAGTTGGTACAGAGCT | |
| | | | | | | GATGCCGTTGGTATTTTT | |
| | | | | | | AAGAAATAATCTTGAGTC | |
| | | | | | | AGAAGGAATGTGCCAAA | |
| | | | | | | CCGGCACAGGTGAGAGA | |
| | | | | | | TCTCTGGACCCCCTTATA | |
| | | | | | | TTTGCC | |
| RASA3 | chr13:114750800- | hypo | 0.63288 | 0.76725 | 92530 | CATCTGCTTTCTGCCTGA | 69 |
| | 114751000 | | | | | CAGAGCCAGGCTTCCTCG | |
| | | | | | | GGACAGCCAGACAGGCG | |
| | | | | | | GCTATTGTTCAAAAGTCA | |
| | | | | | | GGGCCGGTTTGATGGGCT | |
| | | | | | | CTCCCGCCCCCAGTAGAT | |
| | | | | | | GAGCTAGGAAGATGTCA | |
| | | | | | | CTTTTAATTACAACGGTG | |
| | | | | | | AGGGAATAGGTATAGGT | |
| | | | | | | GTGTGCGTGTGTGTGTAT | |
| | | | | | | GGAGCAGGGTTCCACAG | |
| | | | | | | GCTTGT | |
| GRIA1 | chr5:152896400- | hypo | 0.68979 | 0.81569 | 24768 | ACAGTCAGAGCACGGCC | 70 |
| | 152896600 | | | | | TGGCTGCTCATGGGCTGG | |
| | | | | | | AGAGGTTTTCCTGCCCAG | |
| | | | | | | TTGCCTGAGTCACGCTCT | |
| | | | | | | TCCCCCAGGCCCGCCCTT | |
| | | | | | | CCTGCCCTTCACATCTGG | |
| | | | | | | TTCAGGGCTCTGCCGAGG | |
| | | | | | | CCTTCTGGGGCAGGTGCC | |
| | | | | | | AATTATCGCTCAGGAACT | |
| | | | | | | GCTGCTTGCTTCCCAGGG | |
| | | | | | | CTGGCTTTCTGTGAAGGG | |
| | | | | | | ACG | |
| LOC10272 | chr1:7432600- | hypo | 0.86717 | 0.97796 | 17114 | AAACCTCCAGATGGCAC | 71 |
| 5193 | 7432800 | | | | | CAGAGAACTGAGAAAAA | |
| | | | | | | CAATTCCATTGCCAAGGA | |
| | | | | | | GCCTTTGTGTTGCTGACA | |
| | | | | | | AGAGAGGAGCAGGCTGA | |
| | | | | | | GAGAAAACGCCCTGTTTG | |
| | | | | | | TCACAAAGTTACATCTGC | |
| | | | | | | GAGCTCCTTCAAGGAAG | |
| | | | | | | GGAGGTGCCTGACGCTCC | |
| | | | | | | TTGTTAATGCCGGTTGCT | |
| | | | | | | GTTACAGACTTGCAATTG | |
| | | | | | | TGCAAC | |
| HLA- | chr6:33050000- | hypo | 0.74414 | 0.83528 | −1575 | ATGATTATCTGACACGAT | 72 |
| DPA1 | 33050200 | | | | | CCAAGAATCCACAGTGT | |
| | | | | | | AACAGGCTCTATGACTCC | |
| | | | | | | ATTAAAGAAAAGTCAGT | |
| | | | | | | TTAATTCTGTGCTATGCT | |
| | | | | | | CCAGATTACTTCTCTCCT | |
| | | | | | | TTCCTAGATCTCCTCTAT | |
| | | | | | | CAAAGAGAGTAGACGTT | |
| | | | | | | TATAAAATGGTGTCTGAA | |
| | | | | | | TTCAACTCAGTGTCCTGG | |
| | | | | | | AAGCCATATGAAATATTT | |
| | | | | | | TTTTC | |
| MMP17 | chr12:132334600- | hypo | 0.87160 | 0.95124 | 21762 | CAAAAACCCAGGGGCGG | 73 |
| | 132334800 | | | | | GAGGCACATGCGCGTTTT | |
| | | | | | | GGGATCAGGGTGCATCCT | |
| | | | | | | GTTGGTGGAGTGTGACTT | |
| | | | | | | ATGCTACACCAGAGGGG | |
| | | | | | | GTTTGGTGGGAACAGGG | |
| | | | | | | CCCAGGGCAACCACTCA | |
| | | | | | | GGGCCTCGTCTGTCAGGC | |
| | | | | | | CGGGAGACCCGGACTCTT | |
| | | | | | | ACCTGTGGAGATGTGGA | |
| | | | | | | GCTGGGAATGACGCTGTC | |
| | | | | | | CCgaaaa | |
| AFAP1- | chr4:7812800- | hypo | 0.86863 | 0.91366 | 57083 | CTCAGACAGGAAAAACA | 74 |
| AS1 | 7813000 | | | | | GGACCCAGATGGCTCAG | |
| | | | | | | ACTGTTGGGTTAGCATCT | |
| | | | | | | GGCCTTCGACAGGTCACC | |
| | | | | | | CTGGCCCGCCAGATCAGC | |
| | | | | | | CCCAGGCCTGACAAAAG | |
| | | | | | | CACTACAGGATAAAGAA | |
| | | | | | | TAGGATCTCATTAAATGC | |
| | | | | | | AGTGGAAGTAACGTGAG | |
| | | | | | | AAGGATGGCTTTTCATAT | |
| | | | | | | GCCATCCTAACCCTCCCC | |
| | | | | | | TCCCCCC | |
| DTX2P1- | chr7:76624600- | hyper | 0.45024 | 0.43392 | 14561 | ggttgacacaagaacagatgcttgataaa | 75 |
| UPK3BP1- | 76624800 | | | | | tgtttattggataaacgaGCGAAGG | |
| PMS2P11 | | | | | | ATAATCACCTGAGCACCA | |
| | | | | | | TAGTTGCCAAAGCCCCCT | |
| | | | | | | CTAGTCCCAAACCTAAGC | |
| | | | | | | CCACACTCTGTCCCCTTG | |
| | | | | | | CAAGTAAAGAAATGCAC | |
| | | | | | | CAAGCAGGGCTTATGCA | |
| | | | | | | GCAGCCCTCGGTCTGGCC | |
| | | | | | | TGGCTGGAGCTATAAAC | |
| | | | | | | ATGCA | |
| MIR5739 | chr22:28839200- | hyper | 0.24096 | 0.07271 | −16557 | tttgctatgagcaggtttaagttaatattac | 76 |
| | 28839400 | | | | | agaaaggattagcgcaattcctggcaca | |
| | | | | | | gggtgcctgctcagtaaatatgagctattg | |
| | | | | | | ttATTTTACTGCGGGTTCC | |
| | | | | | | CAATGAGTGCCCCACTGT | |
| | | | | | | CACGCACATGCATCCCCC | |
| | | | | | | CGGATCCCACACCCGACC | |
| | | | | | | CAGCTCCCTCTGGATGGG | |
| | | | | | | CACACCACCAGAGTCCTG | |
| | | | | | | TC | |
| PRRX1 | chr1:170635800- | hyper | 0.37076 | 0.19605 | 2628 | AGCCCTTGAAGGGACTA | 77 |
| | 170636000 | | | | | GAAGAGTAAAAACCCTA | |
| | | | | | | GAAAGGACTAGAGAGAT | |
| | | | | | | GCCCCATCCGCCTCATAC | |
| | | | | | | CTGACATATAAGGGTCAC | |
| | | | | | | TGAGAGGCATCCCACCA | |
| | | | | | | ACCCCCTACCTGGCCTAT | |
| | | | | | | AGTCATCTGTGAGGTGGC | |
| | | | | | | AGCCAAAGTTGTAGATG | |
| | | | | | | AGATCGAGGTGACGTCG | |
| | | | | | | CTCCTGTAACCTGATGCC | |
| | | | | | | CACATCTC | |
| TBX15 | chr1:119527600- | hyper | 0.35491 | 0.10532 | 3266 | CTCTCTCCCACTGCAAAC | 78 |
| | 119527800 | | | | | ACCTGGACAAGAACACA | |
| | | | | | | GCCACTGGCAGGCCCAG | |
| | | | | | | CCTGGCTGGTAGGGAGA | |
| | | | | | | ATACATGCTGAGGATAG | |
| | | | | | | ATGCGGAGGTATGGGTG | |
| | | | | | | TGTATGAGGTGCAACCTG | |
| | | | | | | CCAGGACGAACTGTTGC | |
| | | | | | | ATCACAGGGTGAGTCAT | |
| | | | | | | GTTGAGGAACAGCGGCA | |
| | | | | | | GTAGCAGGACCACATCCT | |
| | | | | | | TTCTGGGATC | |
| PTPRN2 | chr7:158362600- | hyper | 0.52325 | 0.17864 | 17815 | CAGGCGTCCCTGAGGCTT | 79 |
| | 158362800 | | | | | CCAGCTGAATCTTTCTGT | |
| | | | | | | CCCTCCTAGGCGTGGCTG | |
| | | | | | | TGGGAATCCTTGGGGCGC | |
| | | | | | | CTTTGGGTTCCAGGAAGC | |
| | | | | | | TGGGGCTGTGTGGGCCG | |
| | | | | | | GGCTGCTAACTGGGGCTG | |
| | | | | | | TGGAGCAATCTGCCAGCC | |
| | | | | | | GAGCCCCTTCCGGAGCTG | |
| | | | | | | TGGGTTCCTCTGGGTGTG | |
| | | | | | | GGGAAGCTGCCCTCCAC | |
| | | | | | | AACC | |
| TNR | chr1:175568400- | hyper | 0.45363 | 0.10538 | 144231 | CTTTGGTTAGCTGCACTG | 80 |
| | 175568600 | | | | | TGCTGAGCGCAGCTCTGC | |
| | | | | | | AGCGAGGTTTAGGGAGC | |
| | | | | | | TGCGGTGCGTTAGGCACA | |
| | | | | | | AGGCACGTGGCGCGCCT | |
| | | | | | | CGCTCCAGCACTTTGCAA | |
| | | | | | | ACGCACAGCAGCGCAGG | |
| | | | | | | GCCAGAGCGGCTGGCCC | |
| | | | | | | CCAGGGGTGGGGGCGAC | |
| | | | | | | CAGGGACAGGAGGTCCC | |
| | | | | | | ATATGGTGTCGTCGCGTT | |
| | | | | | | AAAAACGT | |
| SCUBE3 | chr6:35182400- | hyper | 0.47864 | 0.12249 | 767 | GAGGCCCCAGCTGCACTT | 81 |
| | 35182600 | | | | | TCAGTCACGTGACCTGGT | |
| | | | | | | GACTCAGGCCCTTTCTGG | |
| | | | | | | TTCCTGCTTGTCAGGGTA | |
| | | | | | | TGGGAAAGGTGAGAAAC | |
| | | | | | | TACTTTGCAGTGGCTCTT | |
| | | | | | | TGGGAGGGTCTAGGGAT | |
| | | | | | | GAGCAATAGTTTGTTATT | |
| | | | | | | ATACAGGTTGCCCTGAAC | |
| | | | | | | CCTTTTTATATGAGCATA | |
| | | | | | | ATAAAGTTATCTTATAAC | |
| | | | | | | CCCA | |
| SEPTIN9 | chr17:75369600- | hyper | 0.51492 | 0.05407 | 281 | AATGTGGTGCTAAGTCTA | 82 |
| | 75369800 | | | | | AAGTTTTAACGCATTAAA | |
| | | | | | | GGACAGCATCTGGGGGA | |
| | | | | | | GAAAGGCCAACGCTTTAT | |
| | | | | | | ATTGTCATGACTGTGGTG | |
| | | | | | | AAACTTTTGCTTTCCAAG | |
| | | | | | | TGGAAGCTGTTCCGGTTG | |
| | | | | | | TGCAAAGCCGGATGGCC | |
| | | | | | | TAGGCTGCTGTCTTTCAA | |
| | | | | | | AGGTGGAGAGCATGTGC | |
| | | | | | | GGGTGGGGCCAGGCCGG | |
| | | | | | | GTGGGT | |
| TM2D3 | chr15:102193400- | hypo | 0.59090 | 0.96440 | −906 | ATGGCTCCGCAGGTGAAT | 83 |
| | 102193600 | | | | | TTTCAGCCGACAGGCTTT | |
| | | | | | | ATCATACTGCTTGCTGCA | |
| | | | | | | CCCAGGAAAGGAGCAGG | |
| | | | | | | AAAAGAGCTCTTGTTCCC | |
| | | | | | | TGAAGTGGGCTCGGTTAT | |
| | | | | | | GGGAAAACAGGGCACTC | |
| | | | | | | ACTGTGATAAATGTCTTC | |
| | | | | | | TCACAGCCTGAACAAGG | |
| | | | | | | AAAACACAAACCCTGCT | |
| | | | | | | CAAAGCGTGGCAAAAAT | |
| | | | | | | TATACAA | |
| LOC105374620 | chr5:2917400- | hypo | 0.71770 | 0.97212 | −468 | TCGAGGACAGTTCCGTGT | 84 |
| | 2917600 | | | | | AGAAGTGGAGGCACACA | |
| | | | | | | GTGAAGGCTTTGAGAGG | |
| | | | | | | CTTCGTTAACGGTGCTTT | |
| | | | | | | GAGGGATACATAGGAAG | |
| | | | | | | TATCCGACTCTTTGGGAA | |
| | | | | | | ACACAAAAGCCTTCCTCG | |
| | | | | | | ACATGTCTGTGAGCCAGA | |
| | | | | | | AAAACAAGCAAATGTGA | |
| | | | | | | GAGGTTTCTCAGATCACA | |
| | | | | | | CAGATCTATCCCCTCACT | |
| | | | | | | TACGTA | |
| PIGBOS1 | chr15:55610600- | hypo | 0.72701 | 0.95071 | 621 | TTATTATTATTTTTTGCTT | 85 |
| | 55610800 | | | | | TACTTTCACAGATTGGTG | |
| | | | | | | GTAATGAGTGATTGCTTA | |
| | | | | | | AAGCAATATACATCCACT | |
| | | | | | | TTTTTTGTTGTTGGTTTAT | |
| | | | | | | TGGTTTTGTTAAAACTGT | |
| | | | | | | CTCCAAAGTTTTCACTGA | |
| | | | | | | AACATTTTGAATCACATC | |
| | | | | | | AATACTGTGACGTGTACT | |
| | | | | | | GTGAATAGAAGAGACGG | |
| | | | | | | CCAGGTTTTGGCCAGCAC | |
| | | | | | | T | |
| UNKL | chr16:1430000- | hypo | 0.54246 | 0.71787 | −447 | CTCTAACCATATGTCTGT | 86 |
| | 1430200 | | | | | GTCCACACCTCGTGACAG | |
| | | | | | | GAAACCAGGGTGACAGA | |
| | | | | | | GGAGACAAAGGCGCGGC | |
| | | | | | | AGGAGCAGGGCTGGACG | |
| | | | | | | GGCCTGAAGGAGACCAG | |
| | | | | | | GGGCCCCAAGGTACGAG | |
| | | | | | | TCCACGGCCAGCAAGGC | |
| | | | | | | TTCACTGGGTGACATCTC | |
| | | | | | | TTCCGCCATCTTGTAGCT | |
| | | | | | | TTATGTGGGGCGTGCTTG | |
| | | | | | | GGTTATGA | |
| LBX2-AS1 | chr2:74729000- | hypo | 0.58690 | 0.66709 | −620 | CCTGGAAACACTCACGCT | 87 |
| | 74729200 | | | | | CAAGGAGCGGAGGTGAA | |
| | | | | | | TGGCTTCCTGTTCTCACC | |
| | | | | | | TCCGGGTCCAGGCACTGG | |
| | | | | | | GGGTGAGGAAGTGGCTG | |
| | | | | | | GGAGGCCAGGAGGCTGG | |
| | | | | | | AGGAGGGGAGGTGCTGG | |
| | | | | | | GGCCCAGGGCTGTGCGG | |
| | | | | | | GAGGGGATGGGATCCTG | |
| | | | | | | TGGGTACCTTGGCCCGTG | |
| | | | | | | TGCCAGGGTCGGGGGAA | |
| | | | | | | CGTGACACT | |
| TIMP1 | chrX:47441400- | hypo | 0.34237 | 0.40777 | −335 | CTGACCCGTGGTTCTTGA | 88 |
| | 47441600 | | | | | ACAATTTCAGTGGGCACG | |
| | | | | | | TCTGGCTTTCTTTGTCGA | |
| | | | | | | GTGGAGGCTAAGCTTGC | |
| | | | | | | ACGAGTTAATTTTGTTTT | |
| | | | | | | CTTCCGTGATTTTTTTTTG | |
| | | | | | | AAAGCCGCAGGGCTGCA | |
| | | | | | | CTTGGCGAGTGCTAGAA | |
| | | | | | | AGACGGCCACGTTTGGG | |
| | | | | | | ATCGTGGGCCGGATGGG | |
| | | | | | | GCCTCCGTCGGCCAGGCC | |
| | | | | | | CAGTTG | |
| PSCA | chr8:14375180 | hyper | 0.43386 | 0.37722 | 185 | TCTCCATCATCGCACCTC | 89 |
| | 0-143752000 | | | | | TGATCCTAGAGAAGCCC | |
| | | | | | | AGTCTGTTCTCTTGCTCA | |
| | | | | | | ATCCTGGCCTGAGTCCCT | |
| | | | | | | GGCATCAGCATCCTCTGG | |
| | | | | | | GTCCCAGTCATTCGGCTG | |
| | | | | | | CTCAGCTGTGGCCTCGCT | |
| | | | | | | CGCCTCACTCCACTCGGA | |
| | | | | | | AGGAGGGAAGGGGGTGC | |
| | | | | | | CAGAGAATAGAACTTCTT | |
| | | | | | | GGCCCTAATAGGACGGG | |
| | | | | | | AGAAG | |
| PNMA3 | chrX:152224600- | hyper | 0.38952 | 0.31742 | −127 | TGAAAGCAAAAGAGCCC | 90 |
| | 152224800 | | | | | GGAGACGCTCCCGTGGG | |
| | | | | | | GGCTGGTGTGCGTGTCTG | |
| | | | | | | CAGAGGCTGCCGTGTGA | |
| | | | | | | GCCTCTGTGCAGAGCTTG | |
| | | | | | | GCTGCACCTCAGCTTCCC | |
| | | | | | | GGGCTGCAGAGTGGGCA | |
| | | | | | | CACAGGACACCCATTTCT | |
| | | | | | | CCGAGTGTCCTGTTACGG | |
| | | | | | | GTGCCTCCTCCCGttcttttttttt | |
| | | | | | | tttttttttttgagctg | |
| RPF2 | chr6:111302600- | hyper | 0.68105 | 0.54758 | −520 | GCCGAGACTATCTCAGGT | 91 |
| | 111302800 | | | | | GGCCTTGAACCACTGTTG | |
| | | | | | | GCAAAGGGACAGATAAC | |
| | | | | | | GAGCCCAGGGCAGTGTG | |
| | | | | | | GGGGACTTTGCGTTTTGA | |
| | | | | | | AGTCTGGGTCAGCCAGAT | |
| | | | | | | AGTAAGCATCTTTTGCTT | |
| | | | | | | TTCCTGCTATAACAGATA | |
| | | | | | | TGTGGCCATCCTCCCTCA | |
| | | | | | | GTTGTATATGCAGAGCCT | |
| | | | | | | AAGTTTGCACAGTGTCCT | |
| | | | | | | CCTG | |
| ADCY1 | chr7:45613400- | hyper | 0.54417 | 0.29756 | −239 | GATTTGAGATGGGTAAG | 92 |
| | 45613600 | | | | | GCCTCCATGAATTTCAAC | |
| | | | | | | AATGCATTTAAAAAAAC | |
| | | | | | | AAGCTTTTGTTAAACTTG | |
| | | | | | | GTTAAATCATATACTTAC | |
| | | | | | | AAAGCCACATACAAGAA | |
| | | | | | | CACTAGCTTGACTTAGCA | |
| | | | | | | CAGTCTACTCAGCCCAGA | |
| | | | | | | ACCTGTGAGCTGTGTAGC | |
| | | | | | | TACTACCCCCCGGTGGTG | |
| | | | | | | GGCAAAGATAAGTTCGA | |
| | | | | | | GACAAG | |
| TFAP2A | chr6:10415400- | hyper | 0.41354 | 0.07958 | −193 | ACAGTCAAATACTGGGG | 93 |
| | 10415600 | | | | | ACAGGGAAGTTTCATCTA | |
| | | | | | | GAAAGGTAGGGGTGTAA | |
| | | | | | | GGGGCACCTGCACCCCCT | |
| | | | | | | GGGGCAGCATGCTAACT | |
| | | | | | | CACCTATTTGTGTCTTCA | |
| | | | | | | AGACCCCAGAAATCACA | |
| | | | | | | GCTGGTAGGTGGTCAAG | |
| | | | | | | CAGCAGTGCTCTGGGGC | |
| | | | | | | GATTCTGAGGTAAGGATT | |
| | | | | | | GCAGGCAGAACCAGGGA | |
| | | | | | | GGAAGTTAA | |
| HOXA10- | chr7:27208200- | hyper | 0.45261 | 0.04300 | −218 | tgactcaagccataaactcattctcttcatc | 94 |
| AS | 27208400 | | | | | caGCAAACAAGAGCAGAA | |
| | | | | | | AGTCTTTCACAATCTTTT | |
| | | | | | | TTTTACAATTTACTTTTTA | |
| | | | | | | ACCGCCCTTAATCCAGTT | |
| | | | | | | ATTCCTTAAACTGCCGCC | |
| | | | | | | TTCTATTTACTAATTTCTT | |
| | | | | | | TAAAAAAAAAAAAAGAG | |
| | | | | | | AGAAAGAAAAATATATA | |
| | | | | | | CATTTGCTGCTTCTATAT | |
| | | | | | | CATTGGC | |
| SEPTIN9 | chr17:75369000- | hyper | 0.48086 | 0.04484 | −319 | TCTGCTCCCCCTCCCCCA | 95 |
| | 75369200 | | | | | CAACCCCCAGGAGACCC | |
| | | | | | | CATGTTGGAGAAGCGAC | |
| | | | | | | CCACTGGGCCCCGCCCCG | |
| | | | | | | ACCCCCAGCATTGTTTTC | |
| | | | | | | GGCCCTACGGAGGAGCC | |
| | | | | | | TCATTAGGAAGAGGACG | |
| | | | | | | CAAAAGGGAATCGCTGG | |
| | | | | | | CCCCCAGTCTTGAAAAGC | |
| | | | | | | ACTTCCTTCCCCAAGGGC | |
| | | | | | | TTGGTACCCGTTGGGTGG | |
| | | | | | | GTAGAAT | |
| SEPTIN9 | chr17:75369400- | hyper | 0.47816 | 0.03649 | 81 | GTGAGGGGCGGGGGTGA | 96 |
| | 75369600 | | | | | AAATAATTGGTCAGTTTT | |
| | | | | | | ATGAAGGGCTATCAAGTT | |
| | | | | | | GGCAATGAGTAGCATAG | |
| | | | | | | ACCAAATGTCAACTTTCT | |
| | | | | | | GATCTGATTTCCCAAACG | |
| | | | | | | TACACCCTCAAAACCACA | |
| | | | | | | AGGTAAATAAATAATGC | |
| | | | | | | CTTGAGTCAGCAAAAGG | |
| | | | | | | GGACAAAACATATTTTGA | |
| | | | | | | GAAAAACATTTGAAAGA | |
| | | | | | | ATATTAA | |
| EFNB2 | chr13:107187600- | hyper | 0.50180 | 0.05559 | −312 | CGCCTGTGAGGTGCTTAT | 97 |
| | 107187800 | | | | | CACCTGATACTGCCTCTT | |
| | | | | | | CTTGCTGGCACATCAAGC | |
| | | | | | | TGGAGGATTGCAGCATCT | |
| | | | | | | GAACACGAATGACCAAT | |
| | | | | | | CAAATCTCGGGCCTATAC | |
| | | | | | | CCCACAGACGTAACCTTT | |
| | | | | | | ATCTCCCACCAGAGGGC | |
| | | | | | | ACCAGTAACCTATAAAA | |
| | | | | | | ATCTAAGGGGGCGGGCG | |
| | | | | | | GTCCCTCTTGCCCTTAAC | |
| | | | | | | ATCGTT | |
| SDK1 | chr7:3340400- | hyper | 0.54849 | 0.01933 | −384 | ATCTATCTTCCTAAAAAG | 98 |
| | 3340600 | | | | | CGATGGAAAAAATAACG | |
| | | | | | | GCTTTCAAGAGCAATAGT | |
| | | | | | | AAATTCTTTTTTTTTTTAA | |
| | | | | | | TGGAGTTGTCTATCGGGG | |
| | | | | | | TTACTGATAATTGTCAAC | |
| | | | | | | TGAACCCTTTTGAAACAC | |
| | | | | | | AGGTTTAAATAAACCTTT | |
| | | | | | | TCATAAACATGTCCACTC | |
| | | | | | | TCTCCCTCAGTCCCTCCT | |
| | | | | | | ACCCTCTGATCCTGTTCT | |
| | | | | | | TC | |
| MMP24OS | chr20:33865400-3 | hyper | 0.36846 | 0.07152 | 425 | ttggcactgtcagtctttttcatgttagTT | 99 |
| | 3865600 | | | | | TAGGGTTTATTGCATTGT | |
| | | | | | | TAGAGCTGTTGGGGTAG | |
| | | | | | | GGAAGCTTACAACAACG | |
| | | | | | | AGAATCATTGAAGAAAC | |
| | | | | | | GTGAGTGATGCTCACCTT | |
| | | | | | | GACCTTGCTCAGGGTAAT | |
| | | | | | | CGGCTTAGGAAATTGGCT | |
| | | | | | | TTGAGTATTTCTTGTCCT | |
| | | | | | | CCAGGCAGGAGAGAGGC | |
| | | | | | | TCTGCCAGCAGG | |
| EVX1 | chr7:27280400- | hyper | 0.58965 | 0.25656 | −1921 | gaatggcgccagagtgattcctaaaacc | 100 |
| | 27280600 | | | | | caaatgttattttgcctctactgcttaaactc | |
| | | | | | | ctgtggccgttccctatcttccccagatga | |
| | | | | | | aaaagctcaatatccttagaaaggctgga | |
| | | | | | | aggaccAGCCCAGCTGGTG | |
| | | | | | | CTGCCTGGCTTCCATCTC | |
| | | | | | | CCCGTGAGAAAACGGAG | |
| | | | | | | AGCTGTCTGGTACAAGA | |
| | | | | | | GCTTGCGAGG | |

Table 2 (below) provides exemplary Liver Specific Methylated Targets. Differential methylation regions (DMRs) are reported in average 3 which represents a log ratio of percent DMR. Annotations indicate direction reported as hypermethylated (hyper) or hypomethylated (hypo), chromosome position, mean methylation (average 3) in liver, PBMC (peripheral blood monoculearcytes) and Other Tissues. P-value, false discovery rate (FDR), associated gene, genomic annotation and oligonucleotide sequence are also reported. Duplicated “associated genes,” for example BDH1, are listed multiple times, indicating that there have multiple distinct CpG regions associated with the same gene, but multiple distinct targets can be used. Candidate regions are derived from 100 base pair (bp) binning sequences; however, larger regions can be assessed computationally using by human genome coordinates reported.

Without being bound by theory, it is believed that liver specific patterns that are conserved (remain the same) on liver tissue during varying stages of normal healthy, fibrosis and liver cancer. Without being bound by theory, it is believed that liver specific patterns (while conserved on the liver) can be used as surrogate markers during various stages of disease as they have discrete concentration in circulation; for example, during advanced stages of fibrosis, these liver specific patterns can be higher in circulation as compared to mild fibrosis or no fibrosis. In this example, liver specific methylation patterns can be used as an indirect measurement of excess liver material, such as but not limited to in blood, saliva, urine or stool.

Excess liver specific methylation patterns can indicate disease progression. For example, the concentration of liver specific methylation signatures is stable during a healthy state. In this example, if the concentration of liver specific methylation signatures exceeds healthy status concentration, liver scarring is present (e.g., related to liver fibrosis, cirrhosis, and excess apoptosis).

TABLE 2

| Hyper | | | | | | | | | | SEQ |
|---|---|---|---|---|---|---|---|---|---|---|
| or | Pos- | Mean | Mean | Mean | | | Nearest | Anno- | | ID |
| Hypo | ition | Liver | PBMC | Others | P-value | FDR | Gene | tation | Sequences | NO: |
| hypo | chr1: | 35 | 90 | 87 | 2.00E− | 3.21 E− | AMY1C | Inter- | CAAGCAGCTGG | 101 |
| | 103706600 | | | | 05 | 04 | | genic | GAGCACAGGCA | |
| | | | | | | | | | CGCGACACCAA | |
| | | | | | | | | | GTCCAGCAAATA | |
| | | | | | | | | | TTTTTGTATTTTT | |
| | | | | | | | | | AGTAGAGACGG | |
| | | | | | | | | | GGTCTCACCGTG | |
| | | | | | | | | | TTGGCCAGGCTT | |
| | | | | | | | | | GTCTCTA | |
| hypo | chr1: | 21 | 83 | 82 | 1.01E− | 1.169 | GSTM1 | intron | TTCGTTCATGTG | 102 |
| | 109688900 | | | | 05 | E−04 | | | ACAGTATTCTTA | |
| | | | | | | | | | TTTCAGTCCTGC | |
| | | | | | | | | | CATGAGCAGGC | |
| | | | | | | | | | ACAGTGAGTGCC | |
| | | | | | | | | | CGGTCTCCTCTC | |
| | | | | | | | | | TGCTCTTGCTTA | |
| | | | | | | | | | TGGGAAGGGGA | |
| | | | | | | | | | TGCTGG | |
| hypo | chr1: | 11 | 92 | 86 | 5.88E− | 0.992E- | NOTCH2 | intron | CTCAATTTCCCT | 103 |
| | 120032200 | | | | 05 | 04 | | | ACCTGTACCAAG | |
| | | | | | | | | | CTGAGAGCGAA | |
| | | | | | | | | | CTAAATGTCCCT | |
| | | | | | | | | | GAAGGTTCCCTG | |
| | | | | | | | | | TGATCCACCAGC | |
| | | | | | | | | | GACAGCTCCCTC | |
| | | | | | | | | | CTTATAAGTACA | |
| | | | | | | | | | TGAAT | |
| hypo | chr1: | 27 | 84 | 87 | 6.73E− | 1.08E− | NBPF26 | intron | TTCCAAAACCGA | 104 |
| | 120787300 | | | | 08 | 06 | | | AGAACTTGGGGT | |
| | | | | | | | | | TCGATATTTGAG | |
| | | | | | | | | | GGCAGGAAACA | |
| | | | | | | | | | TCCAGCACGGGA | |
| | | | | | | | | | AAAAGATGTAG | |
| | | | | | | | | | GCTGGGAGGCTA | |
| | | | | | | | | | GGCCAGTCTCGC | |
| | | | | | | | | | TTTTTC | |
| hyper | chr1: | 73 | 23 | 17 | 3.79E− | 0. | PKLR | intron | GCACAGATGGA | 105 |
| | 155294900 | | | | 06 | 00012519 | | | CGTCCGTGGAAG | |
| | | | | | | | | | AAACGACCCACC | |
| | | | | | | | | | CATAGTCCAGCC | |
| | | | | | | | | | CAAACCCAGGGT | |
| | | | | | | | | | GAGCGCAGAGT | |
| | | | | | | | | | CTACACGCTGGG | |
| | | | | | | | | | GACTCCTGGGAC | |
| | | | | | | | | | GGGCTG | |
| hyper | chr1: | 72 | 12 | 10 | 1.68E− | 6.44E− | PKLR | intron | GCAAAAACGCG | 106 |
| | 155295000 | | | | 06 | 05 | | | TGGCCAGGGGA | |
| | | | | | | | | | AGGTGTGATCGG | |
| | | | | | | | | | TCTGAGGGCTGA | |
| | | | | | | | | | TGGGGGAGCCA | |
| | | | | | | | | | AGGAGAAGGGA | |
| | | | | | | | | | ATGTGCCCAGCG | |
| | | | | | | | | | CACGGATGTGGT | |
| | | | | | | | | | CAGGGCGG | |
| hyper | chr1: | 70 | 10 | 7 | 9.69E− | 1.55E− | PKLR | exon | GAGGCGCGTCCG | 107 |
| | 155295100 | | | | 09 | 07 | | | CACCGATTTTCT | |
| | | | | | | | | | GGACCACTAGG | |
| | | | | | | | | | GAGATGAGCCC | |
| | | | | | | | | | GTCGTCAATGTA | |
| | | | | | | | | | GATGCGGCCCCC | |
| | | | | | | | | | CACCGGCACGAC | |
| | | | | | | | | | CCGGACAATATT | |
| | | | | | | | | | GGGGTA | |
| hyper | chr1: | 66 | 12 | 9 | 6.95E− | 1.68E− | PKLR | exon | GGCCCTGGCCCA | 108 |
| | 155295400 | | | | 07 | 05 | | | GCGAGTCCCAGC | |
| | | | | | | | | | CCCACTGCTCAC | |
| | | | | | | | | | CCCCTGCAGGAT | |
| | | | | | | | | | CCCAGTGCGGAT | |
| | | | | | | | | | CTCCGGTCCCTT | |
| | | | | | | | | | GGTGTCCAGGGC | |
| | | | | | | | | | GATGGCCACGG | |
| | | | | | | | | | GCCGG | |
| hyper | chr1: | 61 | 8 | 5 | 2.68E− | 4.48E− | MIR3675 | intron | GCTCGCGGGAGT | 109 |
| | 16694000 | | | | 17 | 16 | | | ACCTCCAGCCCT | |
| | | | | | | | | | CGCGGGGGTACC | |
| | | | | | | | | | TCCAGCCCTCGC | |
| | | | | | | | | | GGGGGTAGCAG | |
| | | | | | | | | | CACGAGCTGGCG | |
| | | | | | | | | | GCCGTCAGCTAA | |
| | | | | | | | | | GGCGGGGAAGA | |
| | | | | | | | | | CAAGAG | |
| hyper | chr1: | 59 | 12 | 7 | 6.82E− | 1.09E− | MIR3675 | intron | AGGGGAGGGAG | 110 |
| | 16694100 | | | | 17 | 15 | | | GCGCGTCTCCCT | |
| | | | | | | | | | CTGGCCCAGCTC | |
| | | | | | | | | | CCGCCGTGGCGC | |
| | | | | | | | | | CAGGAACCTTTT | |
| | | | | | | | | | GCATGTCCTGGG | |
| | | | | | | | | | TGGTGTAATGGC | |
| | | | | | | | | | TCCGCGGGGCTG | |
| | | | | | | | | | CGTCT | |
| hyper | chr1: | 61 | 8 | 7 | 3.96E− | 6.34E− | ESPNP | intron | GCCCCCTGCGCC | 111 |
| | 16700800 | | | | 19 | 18 | | | TCTCCCTAAGCT | |
| | | | | | | | | | GGGAGCTGAGC | |
| | | | | | | | | | CCCCACCTTCAT | |
| | | | | | | | | | CCCCGCCCGAGA | |
| | | | | | | | | | GGAGAGAGGGC | |
| | | | | | | | | | TGACCGTGGGCA | |
| | | | | | | | | | GAGGGGGCCTCT | |
| | | | | | | | | | CATATT | |
| hyper | chr1: | 66 | 5 | 8 | 0. | 0. | ESPNP | intron | ATTTCAGGGGCC | 112 |
| | 16701000 | | | | 00027884 | 00480204 | | | GTAAGGCGGGG | |
| | | | | | | | | | TCGGGTGACAGC | |
| | | | | | | | | | GCGGCTTCCCGC | |
| | | | | | | | | | CCCGTCGCAGCT | |
| | | | | | | | | | GCCCCCAACTAG | |
| | | | | | | | | | GCCCAGCTCAGT | |
| | | | | | | | | | GAGGGAGAGTG | |
| | | | | | | | | | AGGCGG | |
| hyper | chr1: | 58 | 2 | 5 | 0. | 0. | ESPNP | intron | CCGGGCCAAAG | 113 |
| | 16701100 | | | | 00043701 | 00839812 | | | ACTGAGTGACCG | |
| | | | | | | | | | GGTGGGGGCTGT | |
| | | | | | | | | | CCCCTGCCCCAC | |
| | | | | | | | | | TCTCCAGCCCAT | |
| | | | | | | | | | GCGTCCCTGCGG | |
| | | | | | | | | | TGGCCTCAGACC | |
| | | | | | | | | | CTTCACCCCGCC | |
| | | | | | | | | | CGACC | |
| hypo | chr1: | 56 | 97 | 96 | 7.84E− | 0. | MIR4677 | intron | GGTTTGGCCCAC | 114 |
| | 243484000 | | | | 05 | 00129056 | | | CCAGCTAGAGCT | |
| | | | | | | | | | CCAGCTCGGCCC | |
| | | | | | | | | | TTCCTAAGCCCC | |
| | | | | | | | | | AAGTTGCATAGC | |
| | | | | | | | | | TGCCTGGTGGGT | |
| | | | | | | | | | GCGTGTTCCACA | |
| | | | | | | | | | GGTACCTCGGAC | |
| | | | | | | | | | TCGC | |
| hypo | chr10: | 51 | 93 | 90 | 2.74E− | 0. | CHST15 | Inter- | TAGTGAAGCCCA | 115 |
| | 124209600 | | | | 05 | 00045555 | | genic | AGGCTCAGGGGT | |
| | | | | | | | | | TCAACTGGGAAG | |
| | | | | | | | | | CACCGACCTGCT | |
| | | | | | | | | | CTGGGAGGAGA | |
| | | | | | | | | | GGGCTATCTCAC | |
| | | | | | | | | | TGTGGTGGTGTA | |
| | | | | | | | | | GGACCTGGCAG | |
| | | | | | | | | | AATGAA | |
| hypo | chr10: | 13 | 62 | 67 | 3.52E− | 5.69E− | LOC- | Inter- | GAATGGAATGG | 116 |
| | 41903300 | | | | 06 | 05 | 441666 | genic | AATGGAATGGA | |
| | | | | | | | | | AATGAATGGAAT | |
| | | | | | | | | | GGAATGGAATG | |
| | | | | | | | | | GAATGGAATGG | |
| | | | | | | | | | AATGGAACGGA | |
| | | | | | | | | | ATGCAATGGAAT | |
| | | | | | | | | | GGAACAGAACG | |
| | | | | | | | | | GAATGAACCC | |
| hypo | chr10: | 36 | 95 | 94 | 1.61E− | 2.57E− | DNAJC12 | intron | GCTTGAACCCAG | 117 |
| | 67827600 | | | | 07 | 06 | | | GAGGCAGAGGT | |
| | | | | | | | | | TGCAGTGAGCCG | |
| | | | | | | | | | AGATCACGCCAT | |
| | | | | | | | | | TGCACTCCAGCC | |
| | | | | | | | | | TAGACAACAAG | |
| | | | | | | | | | AGTGAAACTCCA | |
| | | | | | | | | | TCTCAAAAAAAA | |
| | | | | | | | | | AAAAAA | |
| hyper | chr11: | 59 | 5 | 9 | 4.38E− | 7.50E− | SNORD131 | Inter- | GTCCCGGTCTCC | 118 |
| | 1969200 | | | | 06 | 05 | | genic | GGGGATGCCGG | |
| | | | | | | | | | CCTCGGCCTGCC | |
| | | | | | | | | | TGGTGGGGGCA | |
| | | | | | | | | | GCAGAGCAGTG | |
| | | | | | | | | | CGGTCGGTCGGA | |
| | | | | | | | | | CGCCTCCCTCTC | |
| | | | | | | | | | CCCGCTTGGGGG | |
| | | | | | | | | | GCCTCCT | |
| hyper | chr11: | 64 | 15 | 13 | 2.40E− | 0. | SNORD131 | Inter- | CGGGCAGCCCCA | 119 |
| | 1969400 | | | | 05 | 00041676 | | genic | CCCTCCTTCCGG | |
| | | | | | | | | | GCAGCCTGCCCT | |
| | | | | | | | | | CCCGGCCCTGGG | |
| | | | | | | | | | CAGGGGGGGCT | |
| | | | | | | | | | CGGGGCGTTGTC | |
| | | | | | | | | | TGATGTCTGAGG | |
| | | | | | | | | | TGGAGGCCGTCT | |
| | | | | | | | | | TCAGG | |
| hypo | chr11: | 48 | 95 | 89 | 9.67E− | 0. | CAPRIN1 | Inter- | TATATATATGTA | 120 |
| | 34026600 | | | | 06 | 00019034 | | genic | TATATGTATATA | |
| | | | | | | | | | CGTATATATCTA | |
| | | | | | | | | | TACATATATACG | |
| | | | | | | | | | TGTATACACGTA | |
| | | | | | | | | | TATACACGTATA | |
| | | | | | | | | | TAGACGTATATA | |
| | | | | | | | | | GACGTATATATA | |
| | | | | | | | | | CGTA | |
| hyper | chr11: | 43 | 2 | 2 | 0. | 0. | LOC- | TTS | GTCCTGGTGGGG | 121 |
| | 555500 | | | | 00016786 | 00268613 | 269247 | | CGAGGGTCGTGA | |
| | | | | | | | | | GGGCGGCGGCC | |
| | | | | | | | | | GGCCCGGGAAA | |
| | | | | | | | | | GGCGGCCCTAGA | |
| | | | | | | | | | GGGCTCCGCGCC | |
| | | | | | | | | | TGGGGCGGGGC | |
| | | | | | | | | | AGGGGCCGCGG | |
| | | | | | | | | | GGCGTACT | |
| hyper | chr11: | 48 | 5 | 5 | 4.23E− | 0. | LOC- | TTS | GCGGCTTGCGGG | 122 |
| | 555800 | | | | 05 | 00067714 | 692247 | | TGTCGGCGCCGG | |
| | | | | | | | | | GCCCGGCCTCAG | |
| | | | | | | | | | GGAGCGGGAAG | |
| | | | | | | | | | CGGTCGATGGAC | |
| | | | | | | | | | AAGTCGGTGCCG | |
| | | | | | | | | | TCGGCGAAGACC | |
| | | | | | | | | | CTCGGGGCCGGA | |
| | | | | | | | | | GTCTC | |
| hyper | chr11: | 66 | 5 | 6 | 2.86E− | 4.81E− | LOC- | exon | CCTTGGGGCTCA | 123 |
| | 556000 | | | | 07 | 06 | 692247 | | GGAGCAGCGTC | |
| | | | | | | | | | GCGCAGCCGCG | |
| | | | | | | | | | GATGGAGAGGA | |
| | | | | | | | | | GGGGAACGGGC | |
| | | | | | | | | | TCCCGGCTCGAG | |
| | | | | | | | | | GACGCGCGCAG | |
| | | | | | | | | | CGGCTTCTTGGC | |
| | | | | | | | | | GCTGCGGGT | |
| hyper | chr11: | 60 | 3 | 4 | 3.69E− | 5.95E− | LOC- | intron | CGCCTCGCCCCA | 124 |
| | 556100 | | | | 08 | 07 | 692247 | | GACCTGGAGGG | |
| | | | | | | | | | GCGTGGAGCGG | |
| | | | | | | | | | CGGGTGAGGGG | |
| | | | | | | | | | CGGCCGGGCCG | |
| | | | | | | | | | GGCCGTCGGGGC | |
| | | | | | | | | | CGGGCTCCCGGG | |
| | | | | | | | | | CCGGGGCGCACC | |
| | | | | | | | | | GTGACGTG | |
| hypo | chr11: | 44 | 94 | 88 | 5.63E− | 0.0018 | FAM86C2P | Interge | CAGGTGATCCCC | 125 |
| | 67863600 | | | | 05 | 3805 | | nic | TGCCTCGGCCTC | |
| | | | | | | | | | CGAAAGTGCTGG | |
| | | | | | | | | | GATTACAGGCGT | |
| | | | | | | | | | GAGCCACCATGC | |
| | | | | | | | | | CTGGCTGGTTTT | |
| | | | | | | | | | GAAGAAGCCAG | |
| | | | | | | | | | CCACATGAGTTT | |
| | | | | | | | | | CACAG | |
| hypo | chr12: | 32 | 94 | 88 | 2.35E− | 0. | C12orf75 | Inter- | CCCTGTAGTCCC | 126 |
| | 105379900 | | | | 05 | 0007849 | | genic | AGCTACTCCGGA | |
| | | | | | | | | | GGCTGAGGCAG | |
| | | | | | | | | | GAGAATGGCGT | |
| | | | | | | | | | GAACCCCAGGG | |
| | | | | | | | | | GGCGGAGCCTGC | |
| | | | | | | | | | AGTGAGCCGAG | |
| | | | | | | | | | ATTGCGCCACTG | |
| | | | | | | | | | CACTCCAG | |
| hypo | chr13: | 36 | 84 | 79 | 0. | 0. | FAM230C | Inter- | CCGTGCCCTGCG | 127 |
| | 18279300 | | | | 00037741 | 0061785 | | genic | GCCTTACCTGGC | |
| | | | | | | | | | TGTTTCACAGTG | |
| | | | | | | | | | ACTTCTGTGCGC | |
| | | | | | | | | | CCAGAAACCTCC | |
| | | | | | | | | | TCTGCCAGCTTG | |
| | | | | | | | | | TACCAGGCGTAG | |
| | | | | | | | | | TTGGGGCTCAGC | |
| | | | | | | | | | AGCC | |
| hypo | chr14: | 17 | 70 | 75 | 1.35E− | 0. | OR11H12 | Inter- | GTTGAGTGAGAC | 128 |
| | 16052800 | | | | 05 | 00021703 | | genic | GCGCTCCTCCCA | |
| | | | | | | | | | CGCGGGGAAGA | |
| | | | | | | | | | GTGCTGCCTGAT | |
| | | | | | | | | | CTGGCGAGCGCA | |
| | | | | | | | | | GGGCCCGTGCTC | |
| | | | | | | | | | CTCTCTGGCGGG | |
| | | | | | | | | | TGCGCGCGGGCC | |
| | | | | | | | | | GTGTG | |
| hypo | chr15: | 18 | 82 | 86 | 1.73E− | 2.76E− | GOLGA8F | intron | CTCACGCTTGCA | 129 |
| | 28397700 | | | | 11 | 10 | | | ATCCCACCACCT | |
| | | | | | | | | | CCGGACGCCGA | |
| | | | | | | | | | GGCGGGCGGAT | |
| | | | | | | | | | AACCTGAAGTGG | |
| | | | | | | | | | GAAGTTTGAGAC | |
| | | | | | | | | | CAGCCTTATGAA | |
| | | | | | | | | | CATGGAGAAAC | |
| | | | | | | | | | CCCATCT | |
| hypo | chr15: | 54 | 95 | 93 | 1.23E− | 1.97E− | LOC10 | Inter- | TTTCAACGTGTT | 130 |
| | 30449200 | | | | 06 | 05 | 0288203 | genic | AGCCAGGAATG | |
| | | | | | | | | | GTCTTGATCTCC | |
| | | | | | | | | | TGATCTCGTGAT | |
| | | | | | | | | | CCGCCTGCCTCG | |
| | | | | | | | | | GCCTCCCAGAGT | |
| | | | | | | | | | GCTGGGATTACA | |
| | | | | | | | | | GGCATGAGCCAC | |
| | | | | | | | | | TGTGC | |
| hyper | chr15: | 42 | 0 | 1 | 6.20E− | 9.92E− | KLF13 | intron | CTCCCTGACCCC | 131 |
| | 31392600 | | | | 16 | 15 | | | TCAGGCCTGTCC | |
| | | | | | | | | | TGGGGGTCCGCC | |
| | | | | | | | | | CTAGAAATAGCT | |
| | | | | | | | | | CGGAGGAAGGA | |
| | | | | | | | | | GCCAGTGACTTG | |
| | | | | | | | | | GGTGACGTTTTG | |
| | | | | | | | | | GAAATTCAGTAG | |
| | | | | | | | | | AGTCT | |
| hyper | chr15: | 63 | 3 | 4 | 9.03E− | 1.44E− | KLF13 | intron | ACTGTCCGGCTC | 132 |
| | 31392900 | | | | 15 | 13 | | | CAGCCAGGTCCG | |
| | | | | | | | | | CTACGCCTGGAG | |
| | | | | | | | | | AGCATGGAGGC | |
| | | | | | | | | | AAGTTCTGCCGG | |
| | | | | | | | | | CTCCACCTGCAA | |
| | | | | | | | | | AGCAGAGCCCCT | |
| | | | | | | | | | TTCCACGACTGT | |
| | | | | | | | | | GCAGC | |
| hyper | chr15: | 71 | 12 | 15 | 2.11E− | 0. | LOC10 | intron | GAGACTCCATCA | 133 |
| | 32613800 | | | | 05 | 00036041 | 1928042 | | CAAAAAAAAAA | |
| | | | | | | | | | AAAAAAAAAGA | |
| | | | | | | | | | GAAAGGAGACG | |
| | | | | | | | | | AAAAACAAGAA | |
| | | | | | | | | | GAGCTCAGCTTT | |
| | | | | | | | | | AAAAAAGGATC | |
| | | | | | | | | | ACGACGTAGAA | |
| | | | | | | | | | AAAGACACAA | |
| hypo | chr15: | 53 | 100 | 91 | 0. | 0. | GOLGA6A | promoter- | TGTCTCTACTCG | 134 |
| | 74083300 | | | | 00030159 | 00532809 | | TSS | AAATACAAAAA | |
| | | | | | | | | | CTAGCCAGGTGT | |
| | | | | | | | | | GGTGGTGGGTAC | |
| | | | | | | | | | CCGTAATCCCAG | |
| | | | | | | | | | CTACTGGGGAGG | |
| | | | | | | | | | CTGAGGCAGGA | |
| | | | | | | | | | GAATCACTTGAA | |
| | | | | | | | | | CCCGGG | |
| hypo | chr16: | 48 | 98 | 94 | 2.85E− | 4.57E− | PDXDC1 | intron | TCCTGACCTCCA | 135 |
| | 14980700 | | | | 09 | 08 | | | CCTCAGCCTCCC | |
| | | | | | | | | | AAAGTTCTGGGA | |
| | | | | | | | | | TTACAGGCATGA | |
| | | | | | | | | | GCCACCGCGCCT | |
| | | | | | | | | | GGCCAATTTTTG | |
| | | | | | | | | | TATTTTTAATAG | |
| | | | | | | | | | AGACGGGGTTTC | |
| | | | | | | | | | GCCA | |
| hypo | chr16: | 39 | 89 | 84 | 0. | 0. | MYH11 | intron | AACCAACATCTG | 136 |
| | 15779000 | | | | 0001677 | 00312708 | | | CTGAGCTGAAAC | |
| | | | | | | | | | CACCCCAGGCCT | |
| | | | | | | | | | GACAGCAACAG | |
| | | | | | | | | | AACGTGTAAAAC | |
| | | | | | | | | | ATGTTCACAGAT | |
| | | | | | | | | | CAATTTCATTCG | |
| | | | | | | | | | GCTTTTGCAATG | |
| | | | | | | | | | ACCGT | |
| hypo | chr16: | 50 | 97 | 93 | 1.34E− | 2.18E− | PDPK1 | intron | ATTACAGGCATG | 137 |
| | 2556500 | | | | 06 | 05 | | | CGCCAACAAGCC | |
| | | | | | | | | | CGGCTAGTTTTT | |
| | | | | | | | | | TTGTATTTTTAG | |
| | | | | | | | | | TAGAGACGGGA | |
| | | | | | | | | | TTTCTCCATGTG | |
| | | | | | | | | | GTCAGGCTGGTC | |
| | | | | | | | | | TCGAACTCCTGA | |
| | | | | | | | | | CCTCA | |
| hypo | chr16: | 31 | 98 | 92 | 1.50E− | 2.47E− | LOC- | intron | TAAAATCTAGAA | 138 |
| | 2615500 | | | | 08 | 07 | 652276 | | TTCCCAGCTGGG | |
| | | | | | | | | | TGCAGTAACTCA | |
| | | | | | | | | | TGCCTAAAACCC | |
| | | | | | | | | | CAGCAGTTTGGG | |
| | | | | | | | | | AGGCCGAGGCG | |
| | | | | | | | | | GGTAGATCACTT | |
| | | | | | | | | | GAGGTCAGGAG | |
| | | | | | | | | | TTCCAC | |
| hypo | chr16: | 41 | 83 | 87 | 7.00E− | 1.18E− | FLJ42627 | TTS | CTCCTGGGCGCA | 139 |
| | 2630900 | | | | 09 | 07 | | | AATGATCGACCT | |
| | | | | | | | | | GCCTCGGCCTCC | |
| | | | | | | | | | CCAAGTGTTGGG | |
| | | | | | | | | | ATTACAGGTGGG | |
| | | | | | | | | | CCACTGTGCCCA | |
| | | | | | | | | | AGCTATTTCACT | |
| | | | | | | | | | ATTTCATCAGCG | |
| | | | | | | | | | CACT | |
| hypo | chr16: | 1 | 72 | 76 | 3.45E− | 7.51E− | CCNYL3 | Inter- | TTGAATGATAAT | 140 |
| | 34892100 | | | | 12 | 11 | | genic | TCTATTCGATTC | |
| | | | | | | | | | CATTGAAAGATT | |
| | | | | | | | | | CCATTCGATGGT | |
| | | | | | | | | | GATTCCATTAGT | |
| | | | | | | | | | GTCCAATCGATG | |
| | | | | | | | | | ATTCCATTCGAT | |
| | | | | | | | | | TCCGTTCGATGA | |
| | | | | | | | | | TGAT | |
| hypo | chr16: | 31 | 77 | 79 | 2.34E− | 3.75E− | LINC | Inter- | CCGGTGGCTCAC | 141 |
| | 36669300 | | | | 06 | 05 | 02167 | genic | GCCTGTAATCCC | |
| | | | | | | | | | AGCACTTTGGGA | |
| | | | | | | | | | GGCCGAGGCGG | |
| | | | | | | | | | GCGGATCACGA | |
| | | | | | | | | | GGTCAGGAGATC | |
| | | | | | | | | | GAGACCATCCCG | |
| | | | | | | | | | GCTAANNNNNN | |
| | | | | | | | | | NNNNNNN | |
| hypo | chr17: | 56 | 96 | 94 | 6.25E− | 1.11E− | SERPINF2 | intron | TCCAGAGGCCAG | 142 |
| | 1745500 | | | | 11 | 09 | | | AAGGGAGAGAG | |
| | | | | | | | | | GGTGGGGAGGA | |
| | | | | | | | | | CCGAAGGTGGG | |
| | | | | | | | | | CGCCAGGCCCCA | |
| | | | | | | | | | GAATGCCAGTGC | |
| | | | | | | | | | CCTCCGTCTGAC | |
| | | | | | | | | | GCTCCCTCTTCC | |
| | | | | | | | | | CTGGGGC | |
| hypo | chr17: | 10 | 94 | 91 | 9.82E− | 1.57E− | KCNJ18 | Inter- | AATGTAATGGAC | 143 |
| | 21913800 | | | | 36 | 34 | | genic | TCTAATAGAATG | |
| | | | | | | | | | GTCTCAAATATA | |
| | | | | | | | | | ATGGACTCGAAA | |
| | | | | | | | | | GAAATTGTCTCG | |
| | | | | | | | | | AATGGAATTTAT | |
| | | | | | | | | | TCAAATGGAATG | |
| | | | | | | | | | GAATCGAATGG | |
| | | | | | | | | | AATGC | |
| hypo | chr17: | 13 | 76 | 73 | 0. | 0. | KCNJ18 | Inter- | AATGGAACTGA | 144 |
| | 21924500 | | | | 00028963 | 00465241 | | genic | ATGGCGTCGAAT | |
| | | | | | | | | | GGAATTGAATCC | |
| | | | | | | | | | AATGGAATGGC | |
| | | | | | | | | | ATTGAATGGAAT | |
| | | | | | | | | | GGAAAGGAATG | |
| | | | | | | | | | GAATGGAATTGA | |
| | | | | | | | | | AAGGAATAGAA | |
| | | | | | | | | | AGGAATGG | |
| hypo | chr17: | 32 | 90 | 84 | 2.43E− | 3.89E− | KCNJ18 | Inter- | GAAAGGAATAG | 145 |
| | 21930500 | | | | 08 | 07 | | genic | AATGGAATGGA | |
| | | | | | | | | | ATGGACCCAAAT | |
| | | | | | | | | | GTAATGGACTCG | |
| | | | | | | | | | AATTGAATGGAC | |
| | | | | | | | | | TCAAATAGAATG | |
| | | | | | | | | | GATTCGAAAGG | |
| | | | | | | | | | AATGGTCTCGAA | |
| | | | | | | | | | TGGAATT | |
| hypo | chr17: | 33 | 89 | 85 | 2.07E− | 3.32E− | KCNJ18 | Inter- | GGCATCGAATGG | 146 |
| | 21945600 | | | | 06 | 05 | | genic | CATCGAATGGAA | |
| | | | | | | | | | TGGAATGGAAA | |
| | | | | | | | | | GAAATAGAATG | |
| | | | | | | | | | GAATGGACCCA | |
| | | | | | | | | | AATATAATGGAC | |
| | | | | | | | | | TCGAATGGAATG | |
| | | | | | | | | | GACTCCAATAGA | |
| | | | | | | | | | ATGGACT | |
| hypo | chr17: | 25 | 93 | 88 | 2.62E− | 4.29E− | UBBP4 | Inter- | GAAACAATAGA | 147 |
| | 21959900 | | | | 11 | 10 | | genic | ATGGAATGGAAT | |
| | | | | | | | | | CGGATGGAATGC | |
| | | | | | | | | | AATGGAATGGA | |
| | | | | | | | | | ATGGAGTCGAGT | |
| | | | | | | | | | GGAATAGAATC | |
| | | | | | | | | | GAATGGAATGG | |
| | | | | | | | | | CATCGAATGGAA | |
| | | | | | | | | | TGTAATTC | |
| hypo | chr17: | 19 | 77 | 75 | 2.33E− | 3.73E− | UBBP4 | Inter- | CGGAATGTAATC | 148 |
| | 21977400 | | | | 14 | 13 | | genic | AAACCGAGTGG | |
| | | | | | | | | | AATGGAATGGA | |
| | | | | | | | | | ATGGAATGAAAT | |
| | | | | | | | | | GGTACGGAATG | |
| | | | | | | | | | GAATCAAGACA | |
| | | | | | | | | | AGTGGAATGGA | |
| | | | | | | | | | ATGGAATGAAAT | |
| | | | | | | | | | GGTATGGAA | |
| hypo | chr17: | 38 | 86 | 79 | 0. | 0. | UBBP4 | Inter- | TGGAATGGAATG | 149 |
| | 21982900 | | | | 00018849 | 00674132 | | genic | GAATCAACCCGA | |
| | | | | | | | | | GTCGAATGGAA | |
| | | | | | | | | | AGGAATGGAAT | |
| | | | | | | | | | GCAATGGACTAT | |
| | | | | | | | | | AATGTGATCAAC | |
| | | | | | | | | | TGGAATGGAATG | |
| | | | | | | | | | GAATGGAATGC | |
| | | | | | | | | | AATGGAA | |
| hypo | chr17: | 53 | 98 | 95 | 3.26E− | 5.22E− | CCL16 | intron | ATCCCAGCTACT | 150 |
| | 35980400 | | | | 08 | 07 | | | CAGGAGGCTGA | |
| | | | | | | | | | GGCAGGAGAAT | |
| | | | | | | | | | CACTTGAACTCG | |
| | | | | | | | | | GGAGGCGGAGG | |
| | | | | | | | | | TTGTGGTGAGCC | |
| | | | | | | | | | GAGATCGCGAC | |
| | | | | | | | | | ACTGCGCTCCAA | |
| | | | | | | | | | CCTGGGCA | |
| hyper | chr17: | 77 | 13 | 14 | 5.33E− | 0. | SRCIN1 | exon | GACTGGGCGGG | 151 |
| | 38564100 | | | | 06 | 0001467 | | | CAGTACCTGGGG | |
| | | | | | | | | | AGCGCAGCTTGG | |
| | | | | | | | | | TCTGGCTGGCCG | |
| | | | | | | | | | AGCGGGAGAGA | |
| | | | | | | | | | GGCAGGCTCTGT | |
| | | | | | | | | | CGGAAGCGGTTC | |
| | | | | | | | | | ATCCTGCTGAAG | |
| | | | | | | | | | CCCAGG | |
| hyper | chr17: | 78 | 2 | 7 | 9.48E− | 2.64E− | SRCIN1 | intron | CCACGCACCTTC | 152 |
| | 38572100 | | | | 12 | 10 | | | CAAGGAGGGAG | |
| | | | | | | | | | GGCAACCCACG | |
| | | | | | | | | | GGTCCACACCGG | |
| | | | | | | | | | CTCCTTAATCCC | |
| | | | | | | | | | CTGGCTGTGCTG | |
| | | | | | | | | | CACCGGTGCACA | |
| | | | | | | | | | CACCCACCTCTC | |
| | | | | | | | | | AACTCC | |
| hyper | chr17: | 72 | 5 | 10 | 2.06E− | 3.30E− | SRCIN1 | intron | CATGAACACGA | 153 |
| | 38572200 | | | | 06 | 05 | | | GTCAGCGTAAAC | |
| | | | | | | | | | ACTCGTCCCCCG | |
| | | | | | | | | | GGCTGGGGCTAA | |
| | | | | | | | | | GCCACTCGCCCC | |
| | | | | | | | | | TCCTCGGGCGCC | |
| | | | | | | | | | TGCCCCCACCGC | |
| | | | | | | | | | GATGTACATGCA | |
| | | | | | | | | | TGACC | |
| hyper | chr17: | 57 | 3 | 2 | 3.95E− | 6.32E− | SRCIN1 | intron | GGGGGAGGGGA | 154 |
| | 38572500 | | | | 25 | 24 | | | GGAGGCGTTTCT | |
| | | | | | | | | | CAGGGATCGGG | |
| | | | | | | | | | AACCTGCAATGG | |
| | | | | | | | | | CCTGGACGTCCC | |
| | | | | | | | | | CCACCCCTGGAT | |
| | | | | | | | | | GGCTCTCGGTGC | |
| | | | | | | | | | CGCGGAGCGGG | |
| | | | | | | | | | CCCCCAT | |
| hyper | chr17: | 66 | 3 | 8 | 1.26E− | 2.08E− | SRCIN1 | intron | TGGAGAGGCGC | 155 |
| | 38578900 | | | | 07 | 06 | | | CCGGGGAGAGG | |
| | | | | | | | | | CAGGGGCTGCCC | |
| | | | | | | | | | AGCACCCCACTA | |
| | | | | | | | | | GGGACTGAAGG | |
| | | | | | | | | | GGGAAGTGCGT | |
| | | | | | | | | | GGATGGCGTGG | |
| | | | | | | | | | AAAGCGCGCCTA | |
| | | | | | | | | | CGGCCCCTA | |
| hypo | chr17: | 29 | 70 | 72 | 3.31E− | 5.30E− | LOC- | Inter- | CTCAGCCTTCTA | 156 |
| | 60123400 | | | | 11 | 10 | 653653 | genic | AAGTGGCGGGA | |
| | | | | | | | | | TTACACGCATGA | |
| | | | | | | | | | GTCACCGCGCCC | |
| | | | | | | | | | AGCCGGGAATG | |
| | | | | | | | | | CATGCTTTTAGG | |
| | | | | | | | | | AGAAACGAAGA | |
| | | | | | | | | | CAACTCAGGCCC | |
| | | | | | | | | | CTCATTC | |
| hyper | chr19: | 67 | 3 | 6 | 1.44E− | 0. | SBNO2 | promoter- | CGCGGCAGCAG | 157 |
| | 1132200 | | | | 05 | 00050049 | | TSS | CCCCAGCATTGG | |
| | | | | | | | | | GTCGGCCGGGGC | |
| | | | | | | | | | GGACGGGGGCG | |
| | | | | | | | | | GCTCTCCGCCCG | |
| | | | | | | | | | GCTGCATTTGCA | |
| | | | | | | | | | TGTCGGTTTAGT | |
| | | | | | | | | | CACCGCCGCCGG | |
| | | | | | | | | | CGCCTA | |
| hypo | chr19: | 35 | 94 | 88 | 4.62E− | 7.57E− | INSR | Inter- | GAACAACCCCA | 158 |
| | 7321200 | | | | 06 | 05 | | genic | GTCATAAAATCA | |
| | | | | | | | | | TCATCTCCGTGA | |
| | | | | | | | | | TCACCACCAGAC | |
| | | | | | | | | | CATCAGCAGCCA | |
| | | | | | | | | | ACCAACCCCCTA | |
| | | | | | | | | | AAACAGCTCCGG | |
| | | | | | | | | | CTTTACAGTTCT | |
| | | | | | | | | | TGGGT | |
| hypo | chr19: | 34 | 94 | 89 | 8.06E− | 2.48E− | CD320 | Inter- | CGATCTCGGCTC | 159 |
| | 8288600 | | | | 07 | 05 | | genic | ACTGCAACACCC | |
| | | | | | | | | | GCTTCCCGGGTC | |
| | | | | | | | | | CAAGCGATCCTC | |
| | | | | | | | | | CTGTCTCAGCCT | |
| | | | | | | | | | CCTGAGAAGCTG | |
| | | | | | | | | | GGATTATAGGCA | |
| | | | | | | | | | CAGGCCACCATG | |
| | | | | | | | | | CCTG | |
| hypo | chr2: | 39 | 96 | 93 | 6.31E− | 1.03E− | MIR | Inter- | ATCTGGCAGGGT | 160 |
| | 110313700 | | | | 34 | 32 | 4436B2 | genic | GTTCGCTGCGCT | |
| | | | | | | | | | TCTGGTCCAGTG | |
| | | | | | | | | | AGGCGCCCACTG | |
| | | | | | | | | | CCGCTCCCGATG | |
| | | | | | | | | | CTGATCGTCGCC | |
| | | | | | | | | | ATTGCAGACCCA | |
| | | | | | | | | | CTGTTGACTTCC | |
| | | | | | | | | | ACCC | |
| hypo | chr2: | 16 | 63 | 64 | 4.76E− | 7.61E− | PRSS40A | intron | GCAGTAGCATTT | 161 |
| | 130549500 | | | | 16 | 15 | | | CCCCATCCTGGA | |
| | | | | | | | | | AATGAGGGTGTG | |
| | | | | | | | | | TGGGGCGCTGGA | |
| | | | | | | | | | CTGTAGAGGCCT | |
| | | | | | | | | | GAGATCCAACCC | |
| | | | | | | | | | GGCAGAGCCAG | |
| | | | | | | | | | CGGGGGAGCGC | |
| | | | | | | | | | CCCGCC | |
| hypo | chr2: | 8 | 88 | 84 | 1.98E− | 0. | ACVR2A | Inter- | AGCAAACATTAT | 162 |
| | 147385500 | | | | 05 | 00032605 | | genic | TAGGTAATATTG | |
| | | | | | | | | | AAATAACGTAAT | |
| | | | | | | | | | AGTTGATCTCCT | |
| | | | | | | | | | GGAAAGGTCAC | |
| | | | | | | | | | CAGCACTACCCA | |
| | | | | | | | | | GAATTACTTATT | |
| | | | | | | | | | GAAATGCCATTT | |
| | | | | | | | | | TTAGG | |
| hypo | chr2: | 11 | 83 | 78 | 8.84E− | 0. | MIR5702 | Inter- | CAATAATTTAAT | 163 |
| | 226515300 | | | | 05 | 0015212 | | genic | CATGATAGTGGT | |
| | | | | | | | | | AAGTACTAAAA | |
| | | | | | | | | | AGGAGAAAAAT | |
| | | | | | | | | | AGCTTGGCAACC | |
| | | | | | | | | | CGGAAACTCTTC | |
| | | | | | | | | | AGAATTAGCCCC | |
| | | | | | | | | | CTTTTTAACCAT | |
| | | | | | | | | | TGTTGT | |
| hyper | chr2: | 62 | 8 | 9 | 2.17E− | 0. | NEU4 | TTS | AGGCTGTGTCTC | 164 |
| | 241804400 | | | | 05 | 00037113 | | | TGTTTAGAACTG | |
| | | | | | | | | | AACACGAGGGT | |
| | | | | | | | | | GGGGAGTGGGG | |
| | | | | | | | | | GATGCTGGACCA | |
| | | | | | | | | | CAGGGCAGCAC | |
| | | | | | | | | | CTGCCCGGCAGG | |
| | | | | | | | | | ATGCGCGTGCCT | |
| | | | | | | | | | GTGAGCC | |
| hypo | chr2: | 18 | 93 | 80 | 5.14E− | 0. | CYTOR | intron | CCATGCCTGGCT | 165 |
| | 87466000 | | | | 05 | 00090754 | | | AATTTTAAAATC | |
| | | | | | | | | | TTTTTTTTGGTA | |
| | | | | | | | | | GAGACGAGCGT | |
| | | | | | | | | | CTCACTGTGTTG | |
| | | | | | | | | | TCCAGGCTGGTC | |
| | | | | | | | | | TCAAACTCCTGT | |
| | | | | | | | | | CCTCCAGTGATC | |
| | | | | | | | | | CTCCC | |
| hypo | chr20: | 28 | 94 | 87 | 3.48E− | 5.61E− | MIR1- | Inter- | CCTGGCAGCCAC | 166 |
| | 62527500 | | | | 08 | 07 | 1HG | genic | TGGTCTTTCTGC | |
| | | | | | | | | | TGTCTCCGTGGT | |
| | | | | | | | | | TTCGTCTTTTCA | |
| | | | | | | | | | AGAGTGTCATAC | |
| | | | | | | | | | CCAGTAAGGCTT | |
| | | | | | | | | | CATTTCATATCA | |
| | | | | | | | | | TTGATAGGTTTT | |
| | | | | | | | | | TGGA | |
| hypo | chr20: | 31 | 92 | 86 | 6.97E− | 1.14E− | MIR1- | non- | AATGCTGAGTTG | 167 |
| | 62543500 | | | | 07 | 05 | 1HG | coding | TCTTACGGCCCT | |
| | | | | | | | | | CCCTGGCAAACC | |
| | | | | | | | | | TGTGACTGGCCC | |
| | | | | | | | | | CGGGACAGTGTC | |
| | | | | | | | | | CTTGGCCAGGAG | |
| | | | | | | | | | GGCAGCCACCCC | |
| | | | | | | | | | CCACCCTGCCTC | |
| | | | | | | | | | TCTC | |
| hypo | chr21: | 50 | 95 | 92 | 7.32E− | 2.44E− | CBS | 5′ | GAGGTAACCGCT | 168 |
| | 43068100 | | | | 08 | 06 | | | CCCTGGACAGCG | |
| | | | | | | | | | GGTGGCTCACAG | |
| | | | | | | | | | CACGTGCTAGGA | |
| | | | | | | | | | CCCGTCCACTGC | |
| | | | | | | | | | TCCTTAGCAGGC | |
| | | | | | | | | | ATGACGCTAACC | |
| | | | | | | | | | CTCCCCACCTGC | |
| | | | | | | | | | TTGC | |
| hypo | chr21: | 52 | 93 | 90 | 1.05E− | 3.52E− | LINC | intron | CCCTCGCACCTA | 169 |
| | 43450000 | | | | 08 | 07 | 00319 | | GGCAGAAGGTC | |
| | | | | | | | | | CATTGCCCTCTG | |
| | | | | | | | | | CCCCGCAGCCTG | |
| | | | | | | | | | CCCGTTCCCAGG | |
| | | | | | | | | | CCAGCGAGGGC | |
| | | | | | | | | | GCTCCCAGGCCG | |
| | | | | | | | | | GTCTGCAGCCAG | |
| | | | | | | | | | AACACA | |
| hyper | chr21: | 50 | 6 | 4 | 1.19E− | 1.95E− | FTCD- | intron | GATAGGAGTTGA | 170 |
| | 46143600 | | | | 10 | 09 | AS1 | | AGGGGCACGCT | |
| | | | | | | | | | AAAAAGTGACC | |
| | | | | | | | | | AAAAAACGAGT | |
| | | | | | | | | | GTGAGCTCTTTG | |
| | | | | | | | | | TCAGGCCTGGGC | |
| | | | | | | | | | AGGGCCACTAG | |
| | | | | | | | | | AGGGCTCCTTGG | |
| | | | | | | | | | TCTAGCGG | |
| hyper | chr21: | 62 | 5 | 4 | 6.95E− | 1.18E− | FTCD- | intron | TAAGGCCAGCGT | 171 |
| | 46143700 | | | | 10 | 08 | AS1 | | CTGGGAAGGCG | |
| | | | | | | | | | CCCGTTACCAAG | |
| | | | | | | | | | CGGAGCATGGTC | |
| | | | | | | | | | TATGGTAGTGTC | |
| | | | | | | | | | AGTGCCAAAGA | |
| | | | | | | | | | AAAGCATCCGCT | |
| | | | | | | | | | ACTTAGCAGATC | |
| | | | | | | | | | GGAAAA | |
| hypo | chr21: | 18 | 100 | 91 | 6.93E− | 1.11E− | LOC10 | intron | AGGCAAGGATT | 172 |
| | 5143500 | | | | 20 | 18 | 2724159 | | GCTTGAACCCAG | |
| | | | | | | | | | GAGTTCAAGACC | |
| | | | | | | | | | AGGCTGGGAAA | |
| | | | | | | | | | ACCCAGCAAGA | |
| | | | | | | | | | CCCGTCTCGACA | |
| | | | | | | | | | AAAAATTTAAAA | |
| | | | | | | | | | ACTAGCCGGGTG | |
| | | | | | | | | | TAGTGGC | |
| hypo | chr21: | 24 | 84 | 69 | 3.23E− | 0. | RNA28SN2 | non- | CGGCGGCGGCG | 173 |
| | 8442000 | | | | 05 | 00091903 | | coding | GTGGCGGCGGC | |
| | | | | | | | | | GGCGGCGGCGG | |
| | | | | | | | | | CGGGACCGAAA | |
| | | | | | | | | | CCCCCCCCGAGT | |
| | | | | | | | | | GTTACAGCCCCC | |
| | | | | | | | | | CCGGCAGCAGC | |
| | | | | | | | | | ACTCGCCGAATC | |
| | | | | | | | | | CCGGGGCCG | |
| hypo | chr22: | 27 | 79 | 79 | 1.58E− | 0. | FRGIFP | Inter- | TATCACACCACC | 174 |
| | 10663900 | | | | 05 | 00025406 | | genic | TAGTCGTTCAAA | |
| | | | | | | | | | TCCTGCTTATTT | |
| | | | | | | | | | GCCCAAGACAA | |
| | | | | | | | | | ATGAAAGCGTAT | |
| | | | | | | | | | GTACAAACGATT | |
| | | | | | | | | | GGACAAACATTC | |
| | | | | | | | | | GTAGCAACTTTA | |
| | | | | | | | | | TTTGA | |
| hypo | chr22: | 45 | 85 | 80 | 3.79E− | 0. | FRG1FP | Inter- | CTCCCTCAGCCT | 175 |
| | 10711100 | | | | 05 | 00068486 | | genic | CCCGAGTAGCTG | |
| | | | | | | | | | GGATTACAGGTA | |
| | | | | | | | | | CGCATCACCACA | |
| | | | | | | | | | CCCAGCTAATTT | |
| | | | | | | | | | TTGGATTTTTAG | |
| | | | | | | | | | TAGAGACTAGGT | |
| | | | | | | | | | TTTACCATGTTG | |
| | | | | | | | | | GCTA | |
| hypo | chr22: | 33 | 90 | 80 | 2.10E− | 0. | CCT8L2 | Inter- | GATTATTTCATT | 176 |
| | 16309300 | | | | 05 | 00050399 | | genic | CGATTCCATTCG | |
| | | | | | | | | | ATGATTCCAATT | |
| | | | | | | | | | GATTCCATTCGA | |
| | | | | | | | | | TAATGATTCCAT | |
| | | | | | | | | | TCGAGTCCATTC | |
| | | | | | | | | | AATTATTCCTTT | |
| | | | | | | | | | TGAGTCCAATCC | |
| | | | | | | | | | ATGA | |
| hypo | chr22: | 30 | 88 | 79 | 6.01E− | 1.04E− | CCT8L2 | Inter- | TTCCATTCGAGT | 177 |
| | 16309400 | | | | 07 | 05 | | genic | CCGTTCGATCAT | |
| | | | | | | | | | TCCATTTGAGTC | |
| | | | | | | | | | CATTCGATGAAG | |
| | | | | | | | | | ATTCCATTCGAG | |
| | | | | | | | | | TCCATTCAATAA | |
| | | | | | | | | | TTCTTTTTAAGT | |
| | | | | | | | | | CCATTTGATGAT | |
| | | | | | | | | | GATT | |
| hypo | chr22: | 2 | 77 | 73 | 5.55E− | 1.74E− | CCT8L2 | Inter- | CGTATCCTTTCA | 178 |
| | 16310700 | | | | 07 | 05 | | genic | ATGATTCCATTT | |
| | | | | | | | | | GATTCCATTCAA | |
| | | | | | | | | | TGATGATTCCAC | |
| | | | | | | | | | TCGAGTCCATTT | |
| | | | | | | | | | GATGATTCCATT | |
| | | | | | | | | | CGATTCCATTTG | |
| | | | | | | | | | ATGATTCCTTTC | |
| | | | | | | | | | GATC | |
| hypo | chr22: | 24 | 91 | 84 | 6.74E− | 1.08E− | CCT8L2 | Inter- | TGGCTTCCATTC | 179 |
| | 16317400 | | | | 07 | 05 | | genic | GATGATGATTCC | |
| | | | | | | | | | TTTCGAATACTT | |
| | | | | | | | | | TCAATGATTCCA | |
| | | | | | | | | | TTCGAGTCCATT | |
| | | | | | | | | | CGATGATGATTC | |
| | | | | | | | | | CATTTGAGTTCA | |
| | | | | | | | | | TTCTATGATTCC | |
| | | | | | | | | | ATTC | |
| hypo | chr22: | 30 | 88 | 81 | 1.61E− | 0. | CCT82L | Inter- | ATTCCATTCGAT | 180 |
| | 16318500 | | | | 05 | 00026234 | | genic | GATTCTGTTTGA | |
| | | | | | | | | | TTCCATTCAATG | |
| | | | | | | | | | ATGATTCCCTTC | |
| | | | | | | | | | TATTCCATTCGA | |
| | | | | | | | | | TGATTCCGTTCG | |
| | | | | | | | | | ATTCCATTCTGT | |
| | | | | | | | | | GATGATTCTATT | |
| | | | | | | | | | CGAT | |
| hypo | chr22: | 42 | 89 | 83 | 1.83E− | 0. | CCT8L2 | Inter- | TTCTATTCGATT | 181 |
| | 16319400 | | | | 05 | 00033704 | | genic | CCATTCAATGAT | |
| | | | | | | | | | GATTCCATCAGA | |
| | | | | | | | | | TTCCATTCGATG | |
| | | | | | | | | | GTTCCATTTGAT | |
| | | | | | | | | | TCCATTCAGTGA | |
| | | | | | | | | | TGGTTCCATTCG | |
| | | | | | | | | | ATTCCATTCGAT | |
| | | | | | | | | | GATG | |
| hypo | chr22: | 49 | 94 | 91 | 1.63E− | 2.70E− | RIMBP3 | Inter- | CCTCCCGAGTAG | 182 |
| | 18588600 | | | | 06 | 05 | | genic | CTGGGACTACAG | |
| | | | | | | | | | GCGCCCGCCACC | |
| | | | | | | | | | ACGCCTGGCTAA | |
| | | | | | | | | | TTTTTTGTATTTT | |
| | | | | | | | | | TAGTAGAGACG | |
| | | | | | | | | | GGGTTTCACCCT | |
| | | | | | | | | | GTTAGCCAGGAT | |
| | | | | | | | | | GGTC | |
| hypo | chr22: | 18 | 94 | 82 | 0. | 0. | RIMBP3 | Inter- | GGCCTGGACAG | 183 |
| | 18593900 | | | | 00011423 | 00183282 | | genic | GCTTAGTGACCC | |
| | | | | | | | | | TTGCACCAGAAC | |
| | | | | | | | | | TCTCCGGACACC | |
| | | | | | | | | | GCCCCTTCGCCC | |
| | | | | | | | | | ACTCGTTAGGCT | |
| | | | | | | | | | CCTCTGCCTTCG | |
| | | | | | | | | | GGCCGGCTGGGC | |
| | | | | | | | | | TGCCC | |
| hypo | chr22: | 31 | 94 | 82 | 1.63E− | 2.63E− | HIC2 | Inter- | CAAGCAATTCTC | 184 |
| | 21414900 | | | | 07 | 06 | | genic | CTGTCTCAGCCT | |
| | | | | | | | | | CCCGAGTAATTG | |
| | | | | | | | | | GGACTACAGGC | |
| | | | | | | | | | ATGCACCTCGAT | |
| | | | | | | | | | ATCCGGCTAATT | |
| | | | | | | | | | TTTGTATTTTTA | |
| | | | | | | | | | GTAGAGACGGG | |
| | | | | | | | | | GTTTCA | |
| hypo | chr22: | 52 | 97 | 96 | 0. | 0. | CYP2D7 | exon | GCCGCTTGCCTT | 185 |
| | 42143400 | | | | 00046328 | 00741795 | | | GGGAACGCGGC | |
| | | | | | | | | | CCGAAGCCCAG | |
| | | | | | | | | | GACCTGGTAGAT | |
| | | | | | | | | | GGGCGCAGGCG | |
| | | | | | | | | | GGCGGTCGGCCG | |
| | | | | | | | | | TGTCCTCGCCGC | |
| | | | | | | | | | GGGTCACCATCG | |
| | | | | | | | | | CCTCGCG | |
| hypo | chr3: | 47 | 92 | 91 | 1.03E− | 2.38E− | LINC | Inter- | AGCAAAACCTCT | 186 |
| | 197417300 | | | | 06 | 05 | 02012 | genic | AATCATTAATTT | |
| | | | | | | | | | TGGTCAAGTTTA | |
| | | | | | | | | | AAACAGAGATTC | |
| | | | | | | | | | CCATTTATGGCG | |
| | | | | | | | | | CACGTCGGTATA | |
| | | | | | | | | | AACACGGCAGCT | |
| | | | | | | | | | TTATAAAAATTC | |
| | | | | | | | | | CCAT | |
| hyper | chr3: | 70 | 4 | 4 | 7.56E− | 1.21E− | BDH1 | intron | GATATATACGCC | 187 |
| | 197554500 | | | | 30 | 28 | | | CTATGCAGAGCC | |
| | | | | | | | | | CGGCTCCAACGC | |
| | | | | | | | | | CTCTTGTAGAAA | |
| | | | | | | | | | GAAGGACACTG | |
| | | | | | | | | | ACCAGGACATTT | |
| | | | | | | | | | GCAGCGTCACAA | |
| | | | | | | | | | AAGATCCTGTCA | |
| | | | | | | | | | GCGCG | |
| hyper | chr3: | 60 | 1 | 1 | 1.33E− | 2.13E− | BDH1 | intron | CCAGGCTGACTG | 188 |
| | 197554700 | | | | 149 | 148 | | | GCTGGAAAAGA | |
| | | | | | | | | | AAAAAAAAAAA | |
| | | | | | | | | | CGCGGAGTTCGA | |
| | | | | | | | | | CGCCGCGCGCAG | |
| | | | | | | | | | CACCAAACACTT | |
| | | | | | | | | | CCCCAGAAGGG | |
| | | | | | | | | | CGCAGCCTGGAG | |
| | | | | | | | | | GCGGCCG | |
| hyper | chr3: | 60 | 2 | 1 | 1.56E− | 2.49E− | BDH1 | intron | CGCGCAGGACG | 189 |
| | 197554800 | | | | 30 | 29 | | | CACGCCCAAGGC | |
| | | | | | | | | | GCCTGGGCCGCT | |
| | | | | | | | | | AGGGACCGACC | |
| | | | | | | | | | GGAGCGCTCAA | |
| | | | | | | | | | ACCCACAGGGTA | |
| | | | | | | | | | TCTATCAGGCGC | |
| | | | | | | | | | GCGTCAGCACGT | |
| | | | | | | | | | AAGCAGA | |
| hyper | chr3: | 66 | 1 | 1 | 7.39E− | 1.18E− | BDH1 | intron | GCTTCCCATTGG | 190 |
| | 197555000 | | | | 47 | 45 | | | TTGAGACAGCAC | |
| | | | | | | | | | CGCCCAGCCAAA | |
| | | | | | | | | | GCCCCCTTGTCC | |
| | | | | | | | | | TCGCGCGGGTGC | |
| | | | | | | | | | GCCGCCTGGACT | |
| | | | | | | | | | CCCACCCTGGCC | |
| | | | | | | | | | AGTCCCGGGCCC | |
| | | | | | | | | | ACCA | |
| hyper | chr3: | 72 | 2 | 2 | 2.58E− | 4.13E− | BDH1 | intron | CCACTCTGGCAT | 191 |
| | 197555100 | | | | 25 | 24 | | | CCCCAGCCTGTC | |
| | | | | | | | | | CCCGCGGCTCCT | |
| | | | | | | | | | CCTCCTTCCCCG | |
| | | | | | | | | | GCCCTGTGGCCT | |
| | | | | | | | | | GCCTGCTGTCCA | |
| | | | | | | | | | GCATTCTTCCCC | |
| | | | | | | | | | AGGGCGCTGTGG | |
| | | | | | | | | | CGCG | |
| hyper | chr3: | 65 | 0 | 0 | 1.84E− | 2.95E− | BDH1 | intron | CTCCTGCCTGGC | 192 |
| | 197555500 | | | | 164 | 163 | | | ACATTATTCCGA | |
| | | | | | | | | | ATAATAAATGCC | |
| | | | | | | | | | AGATCATGCTGC | |
| | | | | | | | | | TGGAGACGCCG | |
| | | | | | | | | | GCAGAAAAGCC | |
| | | | | | | | | | GAGAGGGAGAC | |
| | | | | | | | | | TCCCTGGGGTCT | |
| | | | | | | | | | GGGCGCG | |
| hyper | chr3: | 67 | 1 | 1 | 0 | 0 | BDH1 | intron | ATCGGCCATCCT | 193 |
| | 197555600 | | | | | | | | ACCCTTACCCCG | |
| | | | | | | | | | CTGCGGCCGCAG | |
| | | | | | | | | | GGCTCGGCCTGT | |
| | | | | | | | | | AGGGCGCGAGC | |
| | | | | | | | | | CCCCCTTCCCCA | |
| | | | | | | | | | CCCGTATCCCGG | |
| | | | | | | | | | CTTCGGAGCTAA | |
| | | | | | | | | | AGGAA | |
| hyper | chr3: | 70 | 0 | 1 | 1.56E− | 2.49E− | BDH1 | intron | CACGTGAGGCCG | 194 |
| | 197555700 | | | | 41 | 40 | | | AACCAAACCGA | |
| | | | | | | | | | GTAGAGGGCGC | |
| | | | | | | | | | CAGGGAATCCCG | |
| | | | | | | | | | CCCGAGCTGCAG | |
| | | | | | | | | | AGACGCGGACC | |
| | | | | | | | | | CCTCCTCTTACC | |
| | | | | | | | | | CAAAACCAAAT | |
| | | | | | | | | | GGGCAGGG | |
| hyper | chr3: | 59 | 1 | 1 | 2.99E− | 4.79E− | BDH1 | 5′ | GGCTCCGCTGCA | 195 |
| | 197555800 | | | | 25 | 24 | | | GAGGGCTCGCCG | |
| | | | | | | | | | AGGCTCTCGCCT | |
| | | | | | | | | | TCACCCGGCGCC | |
| | | | | | | | | | GCGCAGGTGCCC | |
| | | | | | | | | | GCTCTCCTGCGG | |
| | | | | | | | | | GGAGAGCCGAG | |
| | | | | | | | | | GGGGGGCGCTG | |
| | | | | | | | | | CCGCAG | |
| hyper | chr3: | 57 | 1 | 2 | 1.63E− | 2.61E− | BDH1 | promoter- | GGGCTCACGGCG | 196 |
| | 197556000 | | | | 10 | 09 | | TSS | GCGTGGCTGTCG | |
| | | | | | | | | | GGGGGCCGCCG | |
| | | | | | | | | | AGCCGCCTCCTC | |
| | | | | | | | | | CGCGCTGGGTGC | |
| | | | | | | | | | CCGGCTGGCGCT | |
| | | | | | | | | | CCTCCCGCCCAA | |
| | | | | | | | | | GGTGCCCTGATC | |
| | | | | | | | | | GCTAG | |
| hyper | chr3: | 55 | 1 | 2 | 5.99E− | 1.46E− | BDH1 | promoter- | GCTCCCGGTGCG | 197 |
| | 197556100 | | | | 25 | 23 | | TSS | GCCGTCTCGCCC | |
| | | | | | | | | | CTAAGCCGGGCG | |
| | | | | | | | | | CCAGATCCCCCG | |
| | | | | | | | | | CCGCCTTCTCGC | |
| | | | | | | | | | TCCCCGACCGCT | |
| | | | | | | | | | GGGCGGGGGCG | |
| | | | | | | | | | AGGGCCAGGGA | |
| | | | | | | | | | GATGGA | |
| hyper | chr3: | 70 | 2 | 2 | 2.87E− | 4.60E− | BDH1 | promoter- | GCCAGGTGCGG | 198 |
| | 197556200 | | | | 40 | 39 | | TSS | AAAGGGTTGCA | |
| | | | | | | | | | GCCTCGCGGGCT | |
| | | | | | | | | | GAGGCCGGAGG | |
| | | | | | | | | | GTGAAGGCGCC | |
| | | | | | | | | | GCTCCTTGAAGT | |
| | | | | | | | | | GAGGAAAACTC | |
| | | | | | | | | | CGGTGAGGAAG | |
| | | | | | | | | | AGGCTCTTTC | |
| hypo | chr3: | 24 | 95 | 92 | 4.10E− | 6.87E− | ZNF860 | intron | TGATTCTTAGAA | 199 |
| | 31966500 | | | | 07 | 06 | | | AAGATCAATAA | |
| | | | | | | | | | ATTGACTGACCT | |
| | | | | | | | | | CTAGCAAAACTG | |
| | | | | | | | | | ATAAAAACAAA | |
| | | | | | | | | | AAGAAACACGA | |
| | | | | | | | | | ACAAGTTACCAA | |
| | | | | | | | | | TATCAGGGAGA | |
| | | | | | | | | | AGAAAAAG | |
| hypo | chr3: | 34 | 82 | 86 | 0. | 0. | LOC10 | Inter- | GAAAAACTGGA | 200 |
| | 3964000 | | | | 70001326 | 0022996 | 0130207 | genic | AACTCTAAAACG | |
| | | | | | | | | | CAGAGCGCCTCT | |
| | | | | | | | | | CCTCCTCCAAAG | |
| | | | | | | | | | GAACGCAGTTCC | |
| | | | | | | | | | TCAACAGCAACA | |
| | | | | | | | | | GAACAAAGCTG | |
| | | | | | | | | | GATGGAGAATG | |
| | | | | | | | | | ATTTTGA | |
| hypo | chr3: | 16 | 78 | 75 | 0. | 0. | GRM7- | Inter- | TTCTCTTCCCTA | 201 |
| | 6379100 | | | | 0004185 | 00693281 | AS3 | genic | AATTGATATTTT | |
| | | | | | | | | | CAGAATGATAG | |
| | | | | | | | | | AGGTTTTCCTTT | |
| | | | | | | | | | TAATTGGTAGAT | |
| | | | | | | | | | TTAAAAAATAAA | |
| | | | | | | | | | TCGGCCTAGATT | |
| | | | | | | | | | CATAGAATAGA | |
| | | | | | | | | | AATGAC | |
| hypo | chr4: | 8 | 85 | 77 | 0. | 0. | FAM241A | intron | CGATCACTGCAG | 202 |
| | 112156200 | | | | 00050482 | 0096254 | | | TAGCATAGATAG | |
| | | | | | | | | | ATCAATTCTATC | |
| | | | | | | | | | TCAGGTCATTTG | |
| | | | | | | | | | CCTGGAACTCTT | |
| | | | | | | | | | CCCCCAGATCTT | |
| | | | | | | | | | CCCTTGGCAGAG | |
| | | | | | | | | | CTTCATATCCCA | |
| | | | | | | | | | GCTT | |
| hypo | chr4: | 45 | 89 | 94 | 9.94E− | 1.62E− | GK3P | intron | AGTTCAAGGCCA | 203 |
| | 165245000 | | | | 10 | 08 | | | GCCTGCCCAACA | |
| | | | | | | | | | TGGTGAAACCTC | |
| | | | | | | | | | ATCTCCACTAAA | |
| | | | | | | | | | ATTACAAAAGTT | |
| | | | | | | | | | AGCCAGGCGTG | |
| | | | | | | | | | GTGGTGCACACT | |
| | | | | | | | | | TGTGGTCCCAGC | |
| | | | | | | | | | TACTC | |
| hypo | chr4: | 15 | 89 | 81 | 6.01E− | 0.0009 | UGT2B4 | exon | TCAGTAGTTTTT | 204 |
| | 69489400 | | | | 05 | 6092 | | | TACACCATTAAA | |
| | | | | | | | | | ACATTTTATCTT | |
| | | | | | | | | | ACCTTTTGTGGG | |
| | | | | | | | | | ATCTTGGCAAGG | |
| | | | | | | | | | GCTGATGCAATT | |
| | | | | | | | | | ACATTGGCCCTT | |
| | | | | | | | | | TCTTCTGACGTG | |
| | | | | | | | | | TTAC | |
| hypo | chr5: | 42 | 95 | 94 | 6.35E− | 1.03E− | MIR548P | intron | AACCTAATGTTG | 205 |
| | 100814700 | | | | 09 | 07 | | | TGTTTGAAATTG | |
| | | | | | | | | | TATAGGCCAATC | |
| | | | | | | | | | TAAATTATTGCA | |
| | | | | | | | | | ATTCGGTATTAA | |
| | | | | | | | | | ACCAGTAACAA | |
| | | | | | | | | | ATTCCACATGTG | |
| | | | | | | | | | AAAAGGACAAG | |
| | | | | | | | | | AATAGA | |
| hypo | chr5: | 25 | 79 | 72 | 6.56E− | 0. | HCN1 | Inter- | CTTACGTGAAGA | 206 |
| | 47076100 | | | | 05 | 00190194 | | genic | TATTTCTTTTTCG | |
| | | | | | | | | | ACCATGGACAA | |
| | | | | | | | | | GAAAGCACTCCA | |
| | | | | | | | | | AATGAACACTTG | |
| | | | | | | | | | GAGATTCTACAA | |
| | | | | | | | | | AAAGTGTGTTTC | |
| | | | | | | | | | AACACTGCTCTA | |
| | | | | | | | | | TCAA | |
| hypo | chr5: | 48 | 100 | 90 | 7.80E− | 0. | HCN1 | Inter- | CAAATCTGTAAG | 207 |
| | 47293100 | | | | 06 | 00012509 | | genic | TCGATATTCGGA | |
| | | | | | | | | | CCTATTAGAGGC | |
| | | | | | | | | | CTTCGTTGGAAA | |
| | | | | | | | | | CGGGATTTCTTC | |
| | | | | | | | | | ATATAATGCTAG | |
| | | | | | | | | | AAAGAAGAATT | |
| | | | | | | | | | CTCAGTAACTTC | |
| | | | | | | | | | CTTGT | |
| hypo | chr5: | 29 | 100 | 89 | 4.91E− | 7.95E− | HCN1 | Inter- | CAACTCACAGAG | 208 |
| | 47300000 | | | | 08 | 07 | | genic | TTGAACGATCGT | |
| | | | | | | | | | TTCCCCAGAGCA | |
| | | | | | | | | | GACTTGAAACAC | |
| | | | | | | | | | TCTTTTTGTGGA | |
| | | | | | | | | | ATTTGCAAATGG | |
| | | | | | | | | | AGATTTCAGCCG | |
| | | | | | | | | | CTTTGAGGTCAA | |
| | | | | | | | | | TGGT | |
| hypo | chr5: | 50 | 96 | 95 | 1.34E− | 2.14E− | LOC10 | Inter- | CGATTCCCCTGC | 209 |
| | 54839500 | | | | 09 | 08 | 2467080 | genic | CTCAGCCTCCTG | |
| | | | | | | | | | AGTAGCTGGGAC | |
| | | | | | | | | | TATAGGCGACCG | |
| | | | | | | | | | CCACCACGCCTG | |
| | | | | | | | | | GCTAATTTTTTTT | |
| | | | | | | | | | TTTTTTTTTTTTT | |
| | | | | | | | | | GTATTTTAGTAC | |
| | | | | | | | | | AG | |
| hypo | chr5: | 30 | 74 | 75 | 6.18E− | 1.01E− | SMA4 | intron | GCGGGATCTCGG | 210 |
| | 70248400 | | | | 18 | 16 | | | CTTACTGCAAAC | |
| | | | | | | | | | TCCGCCTCCCGG | |
| | | | | | | | | | GTTCACGCCATT | |
| | | | | | | | | | CTCCTGCCTCAG | |
| | | | | | | | | | CCTCCCGAGTAG | |
| | | | | | | | | | ATGGGACTAAG | |
| | | | | | | | | | GCGCCCGCCACC | |
| | | | | | | | | | GTGCC | |
| hypo | chr5: | 50 | 92 | 89 | 8.87E− | 0. | C5orf49 | intron | TTTTGTATTTGC | 211 |
| | 7774700 | | | | 05 | 00147176 | | | GCTCGGTTTGTC | |
| | | | | | | | | | TTTGCAATGACC | |
| | | | | | | | | | TTGAAGCTAGGG | |
| | | | | | | | | | AGACTGACTTGA | |
| | | | | | | | | | GAAGCCTCGGCG | |
| | | | | | | | | | ACTTGCCTTCAC | |
| | | | | | | | | | TTCCATCAGTAA | |
| | | | | | | | | | AGCC | |
| hypo | chr5: | 14 | 80 | 82 | 1.29E− | 0. | NBPF22P | Interge | GGATATTAAAAG | 212 |
| | 85440600 | | | | 05 | 00024486 | | nic | GTTTACGTTTAT | |
| | | | | | | | | | ATAAAATGGAC | |
| | | | | | | | | | AAAAATACACAT | |
| | | | | | | | | | TTGCAGTTTGAC | |
| | | | | | | | | | CTCAGGGCTGTT | |
| | | | | | | | | | AAGTTAGCAAAC | |
| | | | | | | | | | TCTTTGTTGACA | |
| | | | | | | | | | TAGTT | |
| hypo | chr5: | 13 | 98 | 93 | 5.78E− | 9.38E− | MIR583 | intron | AATCTACTGTCC | 213 |
| | 96201800 | | | | 13 | 12 | | | TGTCTACTTCTTT | |
| | | | | | | | | | CTCCTTGATGCC | |
| | | | | | | | | | CCCAGGGCCCCA | |
| | | | | | | | | | TCTCCTACGGTA | |
| | | | | | | | | | GGTTGTTTTTAA | |
| | | | | | | | | | TTATTGATTAGA | |
| | | | | | | | | | CAAATCCAACTT | |
| | | | | | | | | | TAA | |
| hypo | chr6: | 28 | 98 | 87 | 1.43E− | 0. | CD24 | Inter- | GAGCTTTAGGAA | 214 |
| | 106897700 | | | | 05 | 00025713 | | genic | GGTCAAATGCCA | |
| | | | | | | | | | CAGCTCCTCCGC | |
| | | | | | | | | | CTCCCGCACCTA | |
| | | | | | | | | | CAATCCAGGAG | |
| | | | | | | | | | AACTGACCTTGA | |
| | | | | | | | | | GCACTGGTCATT | |
| | | | | | | | | | CCAGAGGATTGT | |
| | | | | | | | | | GTAAA | |
| hypo | chr6: | 56 | 97 | 96 | 7.18E− | 1.15E− | ERMARD | intron | GGCATTTGTGTG | 215 |
| | 169767100 | | | | 11 | 09 | | | CGTAACCCCGGG | |
| | | | | | | | | | TAGCGTTCATGT | |
| | | | | | | | | | GCACCTAAAGTG | |
| | | | | | | | | | TGACGACAGCCT | |
| | | | | | | | | | ATGGGAAACCA | |
| | | | | | | | | | AGGGAAGGGTC | |
| | | | | | | | | | ACCTGTAGAGGT | |
| | | | | | | | | | CCGGAC | |
| hypo | chr6: | 6 | 100 | 86 | 3.10E− | 4.96E− | LOC10 | Inter- | GGCTCTGCCACC | 216 |
| | 169905000 | | | | 08 | 07 | 0131532 | genic | TGCCAAGTGTCT | |
| | | | | | | | | | GATCTGAGGACA | |
| | | | | | | | | | AGTGGTGCTTTC | |
| | | | | | | | | | TGGGCCTCAGTG | |
| | | | | | | | | | TCTTCATCTGCA | |
| | | | | | | | | | AAACGGGACCG | |
| | | | | | | | | | TGAGACCACACC | |
| | | | | | | | | | TGACT | |
| hypo | chr6: | 45 | 97 | 90 | 1.18E− | 0. | LOC10 | Inter- | TAGGTTCTGAAG | 217 |
| | 916993100 | | | | 05 | 00018953 | 0131532 | genic | CCTTGGACCAGG | |
| | | | | | | | | | GGACCGGCCGG | |
| | | | | | | | | | AGCTTCTGTGCC | |
| | | | | | | | | | TGAGAATGCTGA | |
| | | | | | | | | | GCCGGTCCAGCA | |
| | | | | | | | | | TGGCCCAGGTCG | |
| | | | | | | | | | ATTGTGCAATCC | |
| | | | | | | | | | TGGGC | |
| hyper | chr6: | 52 | 4 | 4 | 8.95E− | 1.45E− | UBD | Inter- | CCTCTGCCACCT | 218 |
| | 29553400 | | | | 25 | 23 | | genic | GGCCTTGGTAGA | |
| | | | | | | | | | CGCGGGCTTCAC | |
| | | | | | | | | | TACTAGCGTGGT | |
| | | | | | | | | | GCCGCCGCTGCT | |
| | | | | | | | | | GGCCAACCTGCG | |
| | | | | | | | | | CGGACCAGCGCT | |
| | | | | | | | | | CTGGCTGCCGCG | |
| | | | | | | | | | CAGC | |
| hyper | chr6: | 85 | 0 | 9 | 1.05E− | 1.70E− | HCG4B | promoter- | CCTGGACCGCGG | 219 |
| | 29927300 | | | | 11 | 10 | | TSS | CGGACATGGCG | |
| | | | | | | | | | GCTCAGATCACC | |
| | | | | | | | | | AAGCGCAAGTG | |
| | | | | | | | | | GGAGGCAGAAG | |
| | | | | | | | | | AATTTGCAGAGC | |
| | | | | | | | | | AGATCAGGGCCT | |
| | | | | | | | | | ACCTGGAGGGC | |
| | | | | | | | | | ACGTGCGT | |
| hyper | chr6: | 64 | 4 | 12 | 5.55E− | 0. | HCG9 | intron | TGAAAAGCAGG | 220 |
| | 29975600 | | | | 05 | 00089608 | | | GCTGAGGAGTG | |
| | | | | | | | | | GCGGCAACCGG | |
| | | | | | | | | | CAGCGTCCAGCT | |
| | | | | | | | | | CCCGCACCTCGC | |
| | | | | | | | | | TGCACATCGCAC | |
| | | | | | | | | | CTGAGCCCCGCC | |
| | | | | | | | | | GCGACCGCATCG | |
| | | | | | | | | | CGCTCGC | |
| hyper | chr6: | 60 | 6 | 4 | 1.07E− | 1.72E− | TRIM15 | exon | GCTGTTGATGTC | 221 |
| | 30171800 | | | | 10 | 09 | | | TGCGCGCTCCTC | |
| | | | | | | | | | CCTCTAGGGGTC | |
| | | | | | | | | | ATCACTCTGGAC | |
| | | | | | | | | | CCTCAGACCGCC | |
| | | | | | | | | | AGCCGGAGCCTG | |
| | | | | | | | | | GTTCTCTCGGAA | |
| | | | | | | | | | GACAGGAAGTC | |
| | | | | | | | | | AGTGA | |
| hyper | chr6: | 76 | 8 | 7 | 4.52E− | 1.37E− | TRIM15 | exon | GACGGCGTCTGG | 222 |
| | 30172100 | | | | 25 | 23 | | | GCCGTGATCATC | |
| | | | | | | | | | TCGCACCAGCAG | |
| | | | | | | | | | TGCTGGGCCAGC | |
| | | | | | | | | | ACCTCCCCGGGC | |
| | | | | | | | | | ACCGACCTGCCG | |
| | | | | | | | | | CTGAGCGAGATC | |
| | | | | | | | | | CCGCGCGGCGTG | |
| | | | | | | | | | AGAG | |
| hyper | chr6: | 75 | 19 | 18 | 5.65E− | 9.12E− | TRIM15 | 3′ | TTTCTTTGCCGT | 223 |
| | 30172300 | | | | 07 | 06 | | | CTGGAAAAAAG | |
| | | | | | | | | | GTTCCTGCCTTA | |
| | | | | | | | | | CGCTGAAAGGCT | |
| | | | | | | | | | GAAGTGGGGCG | |
| | | | | | | | | | CGCGAAGGGCG | |
| | | | | | | | | | GCGAAGCGGAG | |
| | | | | | | | | | ACGGCGGCTCTC | |
| | | | | | | | | | CGGGATCC | |
| hyper | chr6: | 62 | 3 | 2 | 2.75E− | 4.40E− | HSPA1B | promoter- | CCAAATGCCTTA | 224 |
| | 31826900 | | | | 51 | 50 | | TSS | AGGACGGCCTAC | |
| | | | | | | | | | ATACTAAGGAA | |
| | | | | | | | | | AATTTTTTTCTA | |
| | | | | | | | | | ACTCCTGGTTGC | |
| | | | | | | | | | AGCTGAGGGGA | |
| | | | | | | | | | GCGGCTGAGGG | |
| | | | | | | | | | CGGGGACAGGG | |
| | | | | | | | | | GTGCGGCG | |
| hyper | chr6: | 67 | 4 | 3 | 2.15E− | 4.77E− | B3GALT4 | TTS | CCTCGGAGTACG | 225 |
| | 33277400 | | | | 34 | 33 | | | CCGCACCATGCA | |
| | | | | | | | | | GCTCAGGCTCTT | |
| | | | | | | | | | CCGGCGCCTCCT | |
| | | | | | | | | | TCTCGCCGCTTT | |
| | | | | | | | | | GCTGCTGGTGAT | |
| | | | | | | | | | CGTCTGGACCCT | |
| | | | | | | | | | CTTCGGGCCTTC | |
| | | | | | | | | | GGGG | |
| hypo | chr6: | 29 | 92 | 92 | 1.53E− | 2.49E− | MLIP | intron | TCTGCCTCCCAG | 226 |
| | 54143300 | | | | 10 | 09 | | | GTTCAATTGATT | |
| | | | | | | | | | CTCCTGCCTCAG | |
| | | | | | | | | | CTGGGATTACAG | |
| | | | | | | | | | TCACCTGCCACC | |
| | | | | | | | | | ACACCCAGCTAA | |
| | | | | | | | | | GTTTTGTATTTTT | |
| | | | | | | | | | AGTAGAGACGG | |
| | | | | | | | | | GGTT | |
| hypo | chr6: | 27 | 80 | 74 | 6.80E− | 0. | GUSBP4 | Inter- | GCAGTGGATCTC | 227 |
| | 57680100 | | | | 05 | 00118367 | | genic | CCAGCACAGCGT | |
| | | | | | | | | | TCAAGCTCTGAT | |
| | | | | | | | | | AAGGAACAGAC | |
| | | | | | | | | | TGCGTCCTCAAG | |
| | | | | | | | | | TGAGTCCCTGAC | |
| | | | | | | | | | CCCTGTGTATCC | |
| | | | | | | | | | TGACTGGGAGAC | |
| | | | | | | | | | ACTTC | |
| hypo | chr6: | 34 | 81 | 82 | 9.32E− | 0. | FHL5 | Inter- | ATTCGTTATAAA | 228 |
| | 96672600 | | | | 05 | 00167962 | | genic | ATCCATTAGTTC | |
| | | | | | | | | | TACTGATATTCA | |
| | | | | | | | | | TAATTTCCTTTCT | |
| | | | | | | | | | GAAGAAAAAAC | |
| | | | | | | | | | TATCTGAATAGT | |
| | | | | | | | | | TGGTTTTTTTAG | |
| | | | | | | | | | GCCAACAGTACA | |
| | | | | | | | | | CTGA | |
| hypo | chr7: | 47 | 90 | 90 | 2.74E− | 4.38E− | CUL1 | intron | GGCTCCATAGTG | 229 |
| | 148745600 | | | | 06 | 05 | | | ACTATTAGGATG | |
| | | | | | | | | | AAGATGGACTG | |
| | | | | | | | | | GAGGTAGGGGG | |
| | | | | | | | | | TAGAAAGGAGA | |
| | | | | | | | | | TGTGTTCTCTAT | |
| | | | | | | | | | TCGCAGGAAATC | |
| | | | | | | | | | AATAATTTACGC | |
| | | | | | | | | | AAACGTT | |
| hypo | chr7: | 37 | 83 | 80 | 5.34E− | 0. | ZNF716 | Inter- | AATTCTTTGTGT | 230 |
| | 59938000 | | | | 06 | 00010078 | | genic | AGTGTGCATTCA | |
| | | | | | | | | | ACTCACAGAATG | |
| | | | | | | | | | GAACGTCCCTTT | |
| | | | | | | | | | AGACAGAGCAG | |
| | | | | | | | | | ATTTGAAACACT | |
| | | | | | | | | | CTTTTTGCGCAA | |
| | | | | | | | | | TTTGCAAGTGGA | |
| | | | | | | | | | GATTT | |
| hypo | chr7: | 30 | 75 | 70 | 0. | 0. | LOC10 | Inter- | CACTCTGTCGCC | 231 |
| | 63334200 | | | | 0001633 | 00420135 | 0287704 | genic | CAGGCTGGAGTG | |
| | | | | | | | | | CAGTGGCACGAT | |
| | | | | | | | | | CTCGGCTCACTG | |
| | | | | | | | | | CAACGTCCCCCT | |
| | | | | | | | | | CCCGGGTTCAAG | |
| | | | | | | | | | CGATTCTCCTGC | |
| | | | | | | | | | CTCAGCCTCCTG | |
| | | | | | | | | | AGTA | |
| hypo | chr7: | 28 | 70 | 73 | 6.45E− | 1.29E− | LOC10 | Inter- | CTTGCCCGGTCG | 232 |
| | 63498600 | | | | 07 | 05 | 0287834 | genic | CCCAGGCTGGAG | |
| | | | | | | | | | CGCAGTGGCACA | |
| | | | | | | | | | ATCTCGGCTCAC | |
| | | | | | | | | | TGCAAGCTCCGC | |
| | | | | | | | | | CTCCCGGGTTCA | |
| | | | | | | | | | CGCCATTCTCCT | |
| | | | | | | | | | GCCTCAGCCTCC | |
| | | | | | | | | | CCAG | |
| hypo | chr7: | 52 | 95 | 92 | 1.31E− | 2.09E− | GTF2IR | Inter- | AGCCTCCCACGT | 233 |
| | 74852600 | | | | 06 | 05 | D2 | genic | CAGCCTCCCGAG | |
| | | | | | | | | | TAGGTGGGATCA | |
| | | | | | | | | | CAGGCGCACATC | |
| | | | | | | | | | ACCTCACCCGGC | |
| | | | | | | | | | AAATTTTAAAAA | |
| | | | | | | | | | TTATTTGGCCAG | |
| | | | | | | | | | GCGCAGTGGCTC | |
| | | | | | | | | | ACGC | |
| hypo | chr7: | 20 | 84 | 88 | 9.60E− | 1.58E− | GTF2IR | Inter- | ATCGCCCAGGCT | 234 |
| | 75091200 | | | | 17 | 15 | D2B | genic | GGAGTGCAGTG | |
| | | | | | | | | | GCGTGATCTCGG | |
| | | | | | | | | | CTCACTGCAACC | |
| | | | | | | | | | TTCGCCTCCCAG | |
| | | | | | | | | | GTTCCAGAGATT | |
| | | | | | | | | | CTCCTGTCTCAG | |
| | | | | | | | | | GCTCCCAAGTAG | |
| | | | | | | | | | CTGGG | |
| hypo | chr7: | 50 | 97 | 92 | 3.02E− | 4.96E− | GTF2IP1 | intron | AGCTGGGCATGG | 235 |
| | 75240100 | | | | 10 | 09 | | | TGGTGCCTTCTC | |
| | | | | | | | | | CTGAGGCAGGA | |
| | | | | | | | | | GAATCGCTTGAA | |
| | | | | | | | | | CCCAGGAGGTG | |
| | | | | | | | | | GAGGCTGCAGTA | |
| | | | | | | | | | AGCTGAGATCGC | |
| | | | | | | | | | GCCACTGCACTC | |
| | | | | | | | | | CAGCCT | |
| hypo | chr7: | 38 | 97 | 84 | 0. | 0. | GTF2IP1 | intron | AGCTACAGGCGC | 236 |
| | 75246200 | | | | 00022493 | 00361116 | | | GCGTCATCATGC | |
| | | | | | | | | | CCAGCTAATTTT | |
| | | | | | | | | | TGTATTTTTGGT | |
| | | | | | | | | | AGAGACGGGGT | |
| | | | | | | | | | TTCACCATGTTG | |
| | | | | | | | | | ACCAGGCTTGTC | |
| | | | | | | | | | TCGAACTCCTGA | |
| | | | | | | | | | CCTCA | |
| hypo | chr7: | 31 | 77 | 77 | 0. | 0. | PCLO | Interge | TGAAATATTTTG | 237 |
| | 83309500 | | | | 00027285 | 00536696 | | nic | TAATGTTTTTAC | |
| | | | | | | | | | AATAATAAAATT | |
| | | | | | | | | | TATACACTAAGA | |
| | | | | | | | | | TGCTTAAACGTG | |
| | | | | | | | | | AGTAGTAAAATC | |
| | | | | | | | | | TAGGTATCATTT | |
| | | | | | | | | | TCAAACTAAAAT | |
| | | | | | | | | | ATTA | |
| hypo | chr7: | 32 | 93 | 87 | 1.40E− | 2.26E− | TAC1 | Inter- | CCTGTTTGCCTG | 238 |
| | 97778600 | | | | 06 | 05 | | genic | GGTGTCAGCAGC | |
| | | | | | | | | | AGTGGCTGTAGA | |
| | | | | | | | | | ACAGCGGATTTT | |
| | | | | | | | | | CGTGAGCCACGA | |
| | | | | | | | | | ATGCTGCTGTCT | |
| | | | | | | | | | GATCGTTCCTCT | |
| | | | | | | | | | GGAAGTTTTGTC | |
| | | | | | | | | | GCAG | |
| hypo | chr7: | 41 | 98 | 95 | 6.60E− | 1.06E− | MIR12119 | TTS | GTTTTGTGTGTG | 239 |
| | 99416400 | | | | 07 | 05 | | | TGTTTGTTTTTTG | |
| | | | | | | | | | TTTGTTTGTTTTG | |
| | | | | | | | | | AGATGGAGTCTC | |
| | | | | | | | | | ACTCTGTCACCC | |
| | | | | | | | | | AGGCTGGAGTGC | |
| | | | | | | | | | AGTGGCGCAATC | |
| | | | | | | | | | TTGGCTCACTGC | |
| | | | | | | | | | AA | |
| hypo | chr8: | 53 | 96 | 95 | 1.06E− | 0. | ZHX2 | intron | CCAGCTACTTGG | 240 |
| | 122887400 | | | | 05 | 00018241 | | | GAGGCTGAGGC | |
| | | | | | | | | | AAGAGAATAGC | |
| | | | | | | | | | TTGAACCTGGGA | |
| | | | | | | | | | GGCAGAGGTTGC | |
| | | | | | | | | | AGTGAGCCGAG | |
| | | | | | | | | | ATTGCGCCACTG | |
| | | | | | | | | | CACTCCAGCCTG | |
| | | | | | | | | | GGTGACA | |
| hypo | chr8: | 55 | 97 | 95 | 5.05E− | 8.08E− | GSDMD | exon | CCCGGCACTGAC | 241 |
| | 143561700 | | | | 08 | 07 | | | CAGCCCTGCCCT | |
| | | | | | | | | | CCCATGCCCCTG | |
| | | | | | | | | | CCCAGGCCACAA | |
| | | | | | | | | | GCGTTCCACGAG | |
| | | | | | | | | | CGAAGGCGCCTG | |
| | | | | | | | | | GCCACAGCTGCC | |
| | | | | | | | | | CTCTGGCCTCTC | |
| | | | | | | | | | CATG | |
| hypo | chr8: | 51 | 95 | 93 | 1.42E− | 0. | MROH6 | exon | CTGCCCGGCGCA | 242 |
| | 143567800 | | | | 05 | 00022793 | | | GGGGGTCCTGTG | |
| | | | | | | | | | GACTCCGCAGGT | |
| | | | | | | | | | AGCCCTGGGTCT | |
| | | | | | | | | | GGCTCAGGAAGT | |
| | | | | | | | | | TGGGCACGTGGC | |
| | | | | | | | | | CTGGGTATCGCT | |
| | | | | | | | | | GAACCTGGGGA | |
| | | | | | | | | | CAAAA | |
| hypo | chr8: | 45 | 94 | 90 | 8.98E− | 0. | KBTBD11- | intron | TCTCATCCCAGC | 243 |
| | 1947700 | | | | 06 | 00019389 | OT1 | | ATCCCACCCACT | |
| | | | | | | | | | GTCAGCACCCGC | |
| | | | | | | | | | CCCGGCCCCCCG | |
| | | | | | | | | | CACTGTCCCCAT | |
| | | | | | | | | | ACCCCACCCCGC | |
| | | | | | | | | | TGTCAGCTGCCT | |
| | | | | | | | | | CTCCCGCCCGCC | |
| | | | | | | | | | TCCC | |
| hypo | chr8: | 50 | 96 | 91 | 9.99E− | 0. | ERICH1 | intron | GGACTTTGCCCC | 244 |
| | 645300 | | | | 05 | 00169254 | | | GCAATGATTTCC | |
| | | | | | | | | | TGTCGTTGAAAA | |
| | | | | | | | | | AACTTTCAATCT | |
| | | | | | | | | | AACTTTCCCCCG | |
| | | | | | | | | | AAATGATACAA | |
| | | | | | | | | | GCAGGGTTTATT | |
| | | | | | | | | | TTGGTATTTTTA | |
| | | | | | | | | | ATGTA | |
| hypo | chr8: | 21 | 69 | 74 | 1.36E− | 0. | USP17L8 | TTS | AGTCCCTCCAGA | 245 |
| | 7971400 | | | | 05 | 00027022 | | | GGTTCGGAAGAC | |
| | | | | | | | | | TCACGACCCCAA | |
| | | | | | | | | | AACATGATGTTT | |
| | | | | | | | | | CCCATATGTGGG | |
| | | | | | | | | | CTCATCCTGAGA | |
| | | | | | | | | | CGCAGCCATCAC | |
| | | | | | | | | | TATCCAATTGTC | |
| | | | | | | | | | CCTG | |
| hypo | chr9: | 11 | 92 | 85 | 1.29E− | 2.13E− | GPR21 | intron | TAAGAAAGGAG | 246 |
| | 122998800 | | | | 07 | 06 | | | AAATCACGTCTG | |
| | | | | | | | | | AGGCTGATTTCT | |
| | | | | | | | | | AAGCCCTCAGAT | |
| | | | | | | | | | CTTTAAACAGTC | |
| | | | | | | | | | ATGTGTATTTTA | |
| | | | | | | | | | AATATATTAAGA | |
| | | | | | | | | | GGAAAAATTATA | |
| | | | | | | | | | TTTTT | |
| hypo | chr9: | 36 | 99 | 86 | 0. | 0. | CNTNAP3 | intron | CCATCGCAAAGA | 247 |
| | 39275000 | | | | 00026453 | 00455364 | | | AGCTAAAAACCT | |
| | | | | | | | | | TGAAAAAGGATT | |
| | | | | | | | | | AGACGAATGGCT | |
| | | | | | | | | | AACTAGAATAA | |
| | | | | | | | | | ACAGCGTAGAG | |
| | | | | | | | | | AAGACCTTAAAT | |
| | | | | | | | | | GACCTGATGGAG | |
| | | | | | | | | | CTGAAA | |
| hypo | chr9: | 8 | 83 | 72 | 7.34E− | 1.18E− | FAM242F | Inter- | TGAGACAGAGTC | 248 |
| | 41768300 | | | | 16 | 14 | | genic | TTGCTCTGTCGC | |
| | | | | | | | | | GCAGGCTGGAGT | |
| | | | | | | | | | GCAGTGGCGCG | |
| | | | | | | | | | ATCTCGGCTCAC | |
| | | | | | | | | | TGCAACCTCCAC | |
| | | | | | | | | | CTCCTGGGTTCA | |
| | | | | | | | | | AGAAATTCTCTT | |
| | | | | | | | | | GCCTC | |
| hypo | chr9: | 20 | 75 | 74 | 2.30E− | 3.68E− | FAM242F | Inter- | TTTCGCTTGGCT | 249 |
| | 41858400 | | | | 37 | 36 | | genic | CTCGTGCTCCCT | |
| | | | | | | | | | TGCAGGCCGCCA | |
| | | | | | | | | | TGGTAAGACGTA | |
| | | | | | | | | | CCTTTCACCTTC | |
| | | | | | | | | | AGCCGTGATTGT | |
| | | | | | | | | | GAGGCCTCCCCA | |
| | | | | | | | | | GCCACGTGGAAC | |
| | | | | | | | | | TGTG | |
| hypo | chr9: | 50 | 93 | 88 | 1.16E− | 0. | CNTNAP3B | intron | GGAGGAGGAGG | 250 |
| | 42107900 | | | | 05 | 00018789 | | | TTGCAGTGAGCC | |
| | | | | | | | | | CAGATGGTGCCA | |
| | | | | | | | | | GTACACTCTAGG | |
| | | | | | | | | | CTGGGCGACAG | |
| | | | | | | | | | AGAAAGACTCC | |
| | | | | | | | | | GTCTTAAAAAAA | |
| | | | | | | | | | AAAAAAAGAAA | |
| | | | | | | | | | GAAAGAAA | |
| hypo | chr9: | 50 | 97 | 96 | 5.15E− | 0. | RIC1 | intron | TTTTTGAGATGG | 251 |
| | 5721900 | | | | 05 | 00082751 | | | AATTTTGCTCTT | |
| | | | | | | | | | GTTGTCCAGACT | |
| | | | | | | | | | GGAGTGCAGTG | |
| | | | | | | | | | GCACGATCTCGG | |
| | | | | | | | | | CTCACCACAACC | |
| | | | | | | | | | TCCGCCTCTCGG | |
| | | | | | | | | | GTTCAAGCGATT | |
| | | | | | | | | | CTCCT | |
| hypo | chr9: | 31 | 78 | 78 | 6.21E− | 9.93E− | LOC10 | Inter- | AAAAAAAATTA | 252 |
| | 64566000 | | | | 11 | 10 | 0996643 | genic | ACCGGGTTTTGT | |
| | | | | | | | | | GGCGGGTGCCTG | |
| | | | | | | | | | TAGTCCCAGCTA | |
| | | | | | | | | | CTCGGGAGGCTG | |
| | | | | | | | | | AGGCAGGAGAA | |
| | | | | | | | | | TGGCATGAACCC | |
| | | | | | | | | | GGGAGGTGGAG | |
| | | | | | | | | | CTTGCAG | |
| hypo | chr9: | 40 | 93 | 90 | 9.12E− | 1.46E− | ZNF658 | Inter- | GTTCTGTCACCC | 253 |
| | 66877400 | | | | 09 | 07 | | genic | AGGCTGGAGTGC | |
| | | | | | | | | | AATGGCGCGATC | |
| | | | | | | | | | TCCGCTCACAGC | |
| | | | | | | | | | AACCTCCACCAC | |
| | | | | | | | | | CCGGATTGAAGC | |
| | | | | | | | | | GATTCTCTTGCC | |
| | | | | | | | | | TCAGCCTCCCGA | |
| | | | | | | | | | GTAG | |
| hypo | chr9: | 31 | 94 | 83 | 0. | 0. | FAM74A3 | intron | ACCCACCTTGGC | 254 |
| | 66975000 | | | | 0001761 | 0028951 | | | CTCCTAAAGTGT | |
| | | | | | | | | | AGGGATTACAG | |
| | | | | | | | | | GCGTGAGCCACC | |
| | | | | | | | | | ATGCCCGGCCCC | |
| | | | | | | | | | AAGTCAAGTCTT | |
| | | | | | | | | | TTTCACAGCCTT | |
| | | | | | | | | | GGTTCACCATAA | |
| | | | | | | | | | AACTA | |
| hypo | chr9: | 36 | 84 | 83 | 0. | 0. | LOC109 | Inter- | GGTCGTGGTGGC | 255 |
| | 67489100 | | | | 00012486 | 00200786 | 272370 | genic | AGGCACCTGTAA | |
| | | | | | | | | | TCCCACCTGCTC | |
| | | | | | | | | | TGGAGGCCGAG | |
| | | | | | | | | | ACAGGAGAATC | |
| | | | | | | | | | ACTTGAACCCGG | |
| | | | | | | | | | GACGTGGGCGTT | |
| | | | | | | | | | GCAGTGAGCTGA | |
| | | | | | | | | | GATCGC | |

Table 3 (below) provides exemplary NASH Specific Differentially Methylated Regions (DMR) that exhibit differential methylation compared to control sample (e.g., patient samples with simple steatosis and normal healthy liver tissue). Beta values are reported as hypomethylated or hypermethylated as compared to control. Associated gene, start and end site of coordinates and description of associated gene as reported.

TABLE 3

| Associated | | Methylation | |
|---|---|---|---|
| Gene | Coordinates (hg18) | Status | Description |
| ARHGEF25 | 58,003,801-58,004,000 | Hyper in | GEFT: Rho |
| | | NASH | guanine |
| | | | nucleotide |
| | | | exchange factor |
| | | | 25 |
| NEU4 | 242,749,801-242,750,000 | Hyper in | Neuraminidase |
| | | NASH | 4 |
| PCOLCE | 100,199,801-100,200,000 | Hyper in | Procollagen C- |
| | | NASH | endopeptidase |
| CREB5 | 28,725,201-28,725,400 | Hypo in NASH | CAMP |
| | | | responsive |
| | | | element |
| | | | binding |
| | | | protein 5 |
| CASP8 | 202,122,201-202, 122,400 | Hypo in NASH | Caspase 8 |
| FMN1 | 33,360,201-33,360,400 | Hypo in NASH | Formin 1 |
| ADGRG1 | 57,653,601-57,653,800 | Hypo in NASH | Adhesion G |
| | | | protein-coupled |
| | | | receptor G1 |
| AQP1 | 30,951,401-30,951,600 | Hypo in NASH | Aquaporin 1 |
| | | | (Colton blood |
| | | | group) |
| CD74 | 149,792,201-149,792,400 | Hypo in NASH | CD74 molecule |
| CAVIN2 | 192,711,801-192,712,000 | Hypo in NASH | Caveolae |
| | | | associated |
| | | | protein |
| | | | 2 |

### EXAMPLES

### Example 1—Methylation Enrichment

In some instances, kits described herein can provide an extraction purification kit (e.g., cell free DNA or exosome isolation kit) and probes for detecting HCC and liver specific methylation signatures along with a standard operating procedure to analyze a patient sample of interest.

Prior to analyzing a subject sample, probes can be designed to target methylation patterns of interest for both liver specific and HCC specific methylation patterns. In some instances, a probe design protocol (e.g., outlined by Methyl Primer Express Software™ 1.0) can be used for developing DNA methylation probes for genomic loci of interest. In some instances, a probe for a methylated state and a pair of probes for an unmethylated can be designed and used in, for example, a two-step PCR reaction. In one aspect, primer targets of the probes contain 2-7 CpGs located towards the 3′ end and contain at least 5 thymidines in the sequence to ensure proper bisulfite conversation.²²

A tissue sample can be obtained from patient previously identified as high risk for liver cancer (e.g., a blood sample, urine, saliva or stool). After obtaining a tissue sample, extraction of circulating material (e.g., exosome isolation or cell free DNA isolation from patient blood samples) can be performed for the purpose of downstream methylation analysis. For exosomes, exosome vesicle isolation can follow methods that utilize kits such as, but not limited to, the System Biosciences SmartSEC HT Exosome Vesicle protocol (“SB”).²³

In some instances, exosome purifications can be carried out starting with at least 250 μl of plasma or serum and isolated through size purification in a 96-well plate platform through a 3-step reaction.²³ In this instance, plasma or serum will be added to a SmartSEC HT plate and allowed to sit at room temperature for 30 minutes followed by centrifuging the sample at 500×g for 2 minutes. Serum or plasma will be mixed with SmartSEC HT Isolation Buffer, and centrifuged again at 500×g for 2 minutes for exosome purification. In this instance, the SB technology has the benefits of size exclusion chromatography such as purity, high yield and preservation of extracellular vesicles (EV) integrity through a simple to use and easy workflow.

These methods outlined for exosome isolation can be followed by methylation assessment using whole genome bisulfite sequencing (e.g., post-bisulfate adapter-tagging (PBAT)), targeted methylation sequencing (targeted bisulfite sequencing or methyl sequencing), pyrosequencing, methylation arrays and/or methylation specific PCR.^{22,37-38}

### Example 2—Methylation Enrichment Using Cell-Free DNA

In another example, a tissue sample (e.g., blood sample, urine, saliva or stool) can be obtained from a patient identified as having a high risk for liver cancer. Cell-free DNA can be extracted from the tissue sample for performing downstream methylation analysis. In this example, cfDNA can be purified from plasma or serum using cfDNA kits (e.g., Qiagen's QiaAMP circulating nucleic acid kit). These methods can follow a 4-step protocol including lysing, binding, washing and eluting, which is carried out using QIAamp Mini columns on a vacuum manifold.²⁴

Downstream methylation analysis can be performed using, for example, bisulfite sequencing (e.g., post-bisulfate adapter-tagging (PBAT)), targeted methylation sequencing (targeted bisulfite sequencing or methyl sequencing), pyrosequencing, methylation arrays and or methylation specific PCR. In one instance, targeted methylation sequencing steps can follow next generation sequencing (NGS) protocol for analysis of methylated cytosines (5mC) at the single nucleotide resolution. Primers for loci of interest can be barcoded for downstream analysis. In one instance, bisulfite conversion can take place prior to addition of sequencing adaptors (e.g., Illumina's TruSeq Methyl Capture EPIC Library Prep protocol kit).²⁵ Bisulfite treatment can be performed by converting non-methylated cytosine to uracil (U), followed by reading the uracil (U) as thymine (T) when sequenced. Methylated cytosines are protected from conversion and still read as cytosine (C). Thus, the remaining cytosine residues correspond to methylated (i.e., protected) residues. The bisulfite-treated DNA can be purified, followed by library preparation, PCR amplification, and sequencing on a sequencing system (e.g., NextSeq 500, NextSeq 2000 or NovaSeq 6000 System). The output allows for quantitative analysis of both percent methylation of target oligonucleotide sequences and copies per mL of target DMRs (differentially methylated regions).

Reads (sequence of base pairs) can be aligned to target sequences and filtered for quality score (known as a Q score which is a property to statistically relate the base calling error probability). In one aspect, regions can contain differentially methylated status with 10% similarity to target sequence to meet the quality score. DMR status are reported in average beta from 0.00 to 1.00 which is a log ratio of percent methylation status, regions greater than 0.50 are considered methylated, while regions less than 0.50 are unmethylated. The concentration of target DMRs can be calculated in copies per mL

In some instances, a copy number of greater than 25 copies per mL of one or more liver specific methylation signatures indicates a tumorigenic phenotype. In some instances, the copy number in a tissue sample can be compared to the copy number in a control tissue sample. In some instances, HCC specific DMRs are indicative of a tumorigenic phenotype if the copy number is greater than 25 copies per mL. In some instances, at least two DMRs probes (e.g., one for detecting liver specific methylation signatures and one for detecting HCC specific methylation signatures) can be used to determine the concentration of liver specific methylation signatures and the HCC specific methylation signatures in a patient sample. In this example, if the copy number of the liver specific methylation signatures and HCC specific methylation signatures is above a threshold (e.g., 25 copies per mL), the patient can be diagnosed with, and optionally treated for, early-stage liver cancer.

For example, if a Multiple Target Epigenetic Assay included 15 DMR targets (such as 5 tissue specific probes, 10 HCC specific probes) these targets would each be assessed in a control patient and a patient suspected of having HCC. If patient in question presents with at least 1 HCC specific methylation signature copy number above 25 copies per mL, optionally as compared to a control, and at least 1 liver specific methylation signature above 25 copies per mL (of the same probes), optionally as compared to a control, then the patient can be diagnosed with, and optionally treated for HCC. In this aspect, samples will be normalized by sample volume to a methylated housekeeping gene found in the liver that has a concentration conserved in varying stages of healthy patients, varying stages of fibrosis and/or HCC (liver cancer).

### REFERENCES

1. Xu, J. (2018). Trends in liver cancer mortality among adults aged 25 and over in the United States, 2000-2016. US Department of Health & Human Services, Centers for Disease Control and Prevention, National Center for Health Statistics.
2. Maucort-Boulch, D., et al. (2018). Fraction and incidence of liver cancer attributable to hepatitis B and C viruses worldwide. International journal of cancer, 142(12), 2471-2477.
3. Kew, Michael C. “Hepatocellular carcinoma: epidemiology and risk factors.” Journal of hepatocellular carcinoma vol. 1 115-25. 13 Aug. 2014, doi:10.2147/JHC.S44381
4. Rawla, Prashanth et al. “Update in global trends and aetiology of hepatocellular carcinoma.” Contemporary oncology (Poznan, Poland) vol. 22, 3 (2018): 141-150. doi:10.5114/wo.2018.78941
5. Qi, J., et al. (2013). Circulating microRNAs (cmiRNAs) as novel potential biomarkers for hepatocellular carcinoma. Neoplasma, 60(2), 135.
6. Gabriel, M. T., et al. (2016). Circulating tumor cells: a review of non-EpCAM-based approaches for cell enrichment and isolation. Clinical chemistry, 62(4), 571-581.
7. Zhang, Y., et al. (2012). Circulating tumor cells in hepatocellular carcinoma: detection techniques, clinical implications, and future perspectives. In Seminars in oncology (Vol. 39, No. 4, pp. 449-460). WB Saunders.
8. Bonnomet, A., et al. (2010). Epithelial-to-mesenchymal transitions and circulating tumor cells. Journal of mammary gland biology and neoplasia, 1(2), 261-273.
9. Jia, S., et al. (2017). Clinical and biological significance of circulating tumor cells, circulating tumor DNA, and exosomes as biomarkers in colorectal cancer. Oncotarget, 8(33), 55632.
10. Ratajczak J, et al. Membrane-derived microvesicles: important and underappreciated mediators of cell-to-cell communication. Leukemia. 2006; 20:1487-95.
11. He M., and Zeng Y. Microfluidic Exosome Analysis toward Liquid Biopsy for Cancer. J Lab Autom. 2016; 21:599-608.
12. Sproul, D., et al. (2012). Tissue of origin determines cancer-associated CpG island promoter hypermethylation patterns. Genome biology, 13(10), R84.
13. Han, T. S., et al. (2018). The epigenetic regulation of HCC metastasis. International journal of molecular sciences, 19(12), 3978.
14. Kulis, M., & Esteller, M. (2010). DNA methylation and cancer. In Advances in genetics (Vol. 70, pp. 27-56). Academic Press.
15. Lee, M. H., et al. (2012). Epigenetic control of metastasis-associated protein 1 gene expression by hepatitis B virus X protein during hepatocarcinogenesis. Oncogenesis, 1(9), e25.
16. Cheng, J., et al. Integrative analysis of DNA methylation and gene expression reveals hepatocellular carcinoma-specific diagnostic biomarkers. Genome Med 10, 42 (2018). https://doi.org/10.1186/s13073-018-0548-z
17. Hao, X., et al. (2017). DNA methylation markers for diagnosis and prognosis of common cancers. Proceedings of the National Academy of Sciences, 114(28), 7414-7419.
18. Lokk, K., et al. (2014). DNA methylome profiling of human tissues identifies global and tissue-specific methylation patterns Genome biology, 15(4), 1-14
19. Tang, W., et al. (2018). Tumor origin detection with tissue-specific miRNA and DNA methylation markers. Bioinformatics, 34(3), 398-406.
20. Tavormina, J. (2019). Identification And Molecular Analysis Of DNA In Exosomes.
21. Lehmann-Werman, R., et al. (2016). Identification of tissue-specific cell death using methylation patterns of circulating DNA. Proceedings of the National Academy of Sciences, 113(13), E1826-E1834.
22. Šestáková, Š., et al. (2019). DNA Methylation Validation Methods: a Coherent Review with Practical Comparison. Biological procedures online, 21(1), 19
23. SBI. “SmartSEC® HT EV Isolation System for Serum & Plasma.” SmartSEC® HT EV Isolation Systemfor Serum & Plasma User Manual, System Biosciences, 16 Jul. 2019, Cat #SSEC096A-1, SSEC008A-SAM.
24. “QIAamp Circulating Nucleic Acid Kit.” QIAamp Circulating Nucleic Acid Kit—QIAGEN Online Shop, www.qiagen.com/us/products/discovery-and-translational-research/dna-rna-purification/rna-purification/cell-free-rna/qiaamp-circulating-nucleic-acid-kit/.
25. “TruSeq Methylcapture EPIC Library Prep Kit,” Illumina Online Shup. Retrieved at https://www.illumina.com/products/by-type/sequencing-kits/library-prep-kits/truseq-methyl-capture-epic.html
26. TCGA Research Network. Retrieved from: https://www.cancer.gov/tcga.
27. Edgar et al., 2002. Data Discussed in this publication have been deposited in NCBI's Gene Expression Omnibus and are accessible through GEO Series accession number GSE and link
28. Aryee, Martin J, et al. 2014. “Minfi: a flexible and comprehensive Bioconductor package for the analysis of Infinium DNA methylation microarrays.” Bioinformatics 30
29. Jaffe, Andrew E, et al. 2012 “Bump Hunting to identify Differentially Methylated Regions in Epigenetic Epidemiology Studies.” International Journal of Epidemiology 41 (1): 200-209. https://doi.org/10.1093/ije/dyr238.
30. A. Liaw and M. Wiener (2002). Classification and Regression by randomForest. R News 2(3), 18-22.
31. Drescher, H. K., et al. (2019). Current status in testing for nonalcoholic fatty liver disease (NAFLD) and nonalcoholic steatohepatitis (NASH). Cells, 8(8), 845.
32. Arulanandan, A., & Loomba, R. (2015). Noninvasive testing for NASH and NASH with advanced fibrosis: are we there yet?. Current hepatology reports, 14(2), 109-118. Non Invasive
33. Piazzolla, V. A., & Mangia, A. (2020). Noninvasive Diagnosis of NAFLD and NASH. Cells, 9(4), 1005.
34. Younossi, Z. M., et al. (2016). The economic and clinical burden of nonalcoholic fatty liver disease in the United States and Europe. Hepatology, 64(5), 1577-1586,
35. Vilar-Gomez, E., et al. (2020). Cost effectiveness of different strategies for detecting cirrhosis in patients with nonalcoholic fatty liver disease based on United States health care system. Clinical Gastroenterology and Hepatology. 18(10), 2305-2314.
36. Hlady, R. A., et al. (2017). Initiation of aberrant DNA methylation patterns and heterogeneity in precancerous lesions of human hepatocellular cancer. Epigenetics, 12(3), 215-225.
37. Ziller, M. J., et al. (2015). Coverage recommendations for methylation analysis by whole-genome bisulfite sequencing. Nature methods, 12(3), 230-232.
38. Hughes, S., & Jones, J. L. (2007). The use of multiple displacement amplified DNA as a control for methylation specific PCR, pyrosequencing, bisulfite sequencing and methylation-sensitive restriction enzyme PCR. BMC molecular biology, 8(1), 1-7.
39. Bernstein, B. E., et al. (2010). The NIH roadmap epigenomics mapping consortium. Nature biotechnology, 28(10), 1045-1048.
40. Stunnenberg, H. G, et al. (2016). The International Human Epigenome Consortium: a blueprint for scientific collaboration and discovery. Cell, 167(5), 1145-1149.

Various aspects have been described above. Although these aspects have been described with reference to specific examples, the descriptions are provided for illustrative purposes and are not intended to be limiting. Those skilled in the art will recognize or be able to ascertain using no more than routine experimentation, many equivalents to the specific embodiments described herein. Such equivalents are intended to be encompassed by the following claims. For instance, various modifications and applications may occur to those skilled in the art without departing from the scope of the invention as defined by the following claims.

Patents, patent applications, and printed publications cited herein are incorporated by reference in their entirety.

1.-82. (canceled)

83. A method of determining if an HCC specific methylation signature is detected in a subject suspected of having liver cancer, comprising:
(a) obtaining a tissue sample comprising a nucleic acid from the subject;
(b) isolating the nucleic acid from the tissue sample;
(c) determining a concentration of the HCC-specific methylation signature in the tissue sample by contacting the nucleic acid with at least one HCC-specific methylation signature detection molecule, wherein the HCC-specific methylation signature detection molecule is a probe comprising at least 5 nucleotides of a nucleotide sequence of any one of SEQ ID NOs:1-100; and
(d) determining if the HCC-specific methylation signature is detected in the tissue sample.

84. The method of claim 83, wherein the concentration of the HCC-specific methylation signature concentration exceeds about 25 copies per milliliter (mL) in the tissue sample.

85. The method of claim 83, wherein the tissue sample is selected from the group consisting of blood, urine, stool, liver, and lymph.

86. The method of claim 84, wherein the subject at high risk for liver cancer is selected from the group consisting of a subject having viral hepatitis, obesity, diabetes, polycystic ovary syndrome, metabolic syndrome, non-alcoholic fatty liver disease (NAFLD), fibrosis, cirrhosis, and/or chronic liver disease.

87. The method of claim 83, wherein the tissue sample is from a subject who has not been diagnosed with liver cancer.

88. The method of claim 83, wherein the HCC-specific methylation signature comprises at least 2 to 7 CpG marker sites.

89. The method of claim 83, further comprising extracting a circulating material from the tissue sample prior to (b).

90. The method of claim 89, wherein the circulating material is extracted from the tissue sample by exosome isolation or cell-free DNA isolation.

91. The method of claim 83, wherein the at least one HCC-specific methylation signature is associated with one or more of POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEG5, MROH5, MRPL36, LINC00602, MEGF6, MIR4456, MIR6072, LINC02667, MIR4472-1, EFNA5, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS 1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP240S, and EVX1.

92. A kit comprising at least one HCC-specific methylation signature detection molecule capable of binding to one or more methylation patterns associated with POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINCO2500, SMOC2, MIR4689, THEGS, MROHS, MRPL36, LINC00602, MEGF6, MIR4456, MIR6072, LINCO2667, MIR4472-1, EFNAS, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1;
wherein the liver-specific methylation signature detection molecule is a probe comprising at least 5 nucleotides of a nucleotide sequence of any one of SEQ ID NOs:1-100.

93. A method of detecting at least one HCC-specific methylation signature in a tissue sample of a subject, comprising:
contacting a HCC-specific methylation signature detection molecule with a nucleic acid obtained from the tissue sample;
wherein the HCC-specific methylation signature detection molecule is capable of binding to one or more methylation patterns associated with POM121L12, CSMD3, AJAP1, COTL1, TRIL, SLC25A36, TMEM51-AS1, TACC2, NUDT16L1, NLRP2, MIR7160, MYL1, LOC391322, EGR3, PCDHGA12, GAS1, CCDC177, SIX2, BASP1P1, ANKRD30A, TMEM132D, LINC02500, SMOC2, MIR4689, THEGS, MROHS, MRPL36, LINC00602, MEGF6, MIR4456, MIR6072, LINC02667, MIR4472-1, EFNAS, LONRF3, TBC1D28, NLGN4Y, USP3, UTP14A, FOXD1, SLC22A31, PRRX1, LINC01381, ACTG1, HOXA11-AS, FOXC1, DUSP10, LOC101929268, ENGASE, DPP10, LSAMP-AS1, DLGAP2-AS1, PXDN, LINC01103, WWOX, PBX1, LINC02645, LINC00200, VCX3A, ZFPM2, COL14A1, LINC01587, MIR561, AJAP1, RIMS1, OCA2, PBX1, MIR4251, RASA3, GRIA1, LOC102725193, HLA-DPA1, MMP17, AFAP1-AS1, DTX2P1-UPK3BP1-PMS2P11, MIR5739, PRRX1, TBX15, PTPRN2, TNR, SCUBE3, SEPTIN9, TM2D3, LOC105374620, PIGBOS1, UNKL, LBX2-AS1, TIMP1, PSCA, PNMA3, RPF2, ADCY1, TFAP2A, HOXA10-AS, SEPTIN9, SEPTIN9, EFNB2, SDK1, MMP24OS, and EVX1; and
wherein the HCC-specific methylation signature detection molecule is a probe comprising at least 5 nucleotides of a nucleotide sequence of any one of SEQ ID NOs:1-100.

94. The method of claim 93, wherein the tissue sample is selected from the group consisting of blood, stool, urine, liver, and lymph.
